(19) United States
(12) Patent Application Publication
(10) Pub. No.: US 2023/0330143 A1
(43) Pub. Date: Oct. 19, 2023

(54) IMMUNOTHERAPY COMPOSITION

(71) Applicant: GAMMADELTA THERAPEUTICS LIMITED, White City Place, London (GB)

(72) Inventors: Timothy RECALDIN, White City Place, London (GB); Andre SIMOES, White City Place, London (GB); Oliver NUSSBAUMER, White City Place, London (GB); Istvan KOVACS, White City Place, London (GB); Mihil PATEL, White City Place, London (GB)

(73) Assignee: GAMMADELTA THERAPEUTICS LIMITED, White City Place, London (GB)

(21) Appl. No.: 18/044,195

(22) PCT Filed: Sep. 4, 2021
(86) PCT No.: PCT/IB2021/058083

§ 371 Date: Mar. 6, 2023

(30) Foreign Application Priority Data
Sep. 4, 2020 (GB) 2013962.2

(51) Int. Cl. A61K 35/17; C12N 5/0783; C07K 16/28

(52) U.S. Cl. CPC A61K 35/17; C12N 5/0636; C07K 16/2809; C12N 2501/2315; C12N 2501/515; C12N 2502/1164

(57) ABSTRACT

The invention relates to compositions comprising NaturalKiller (NK) cells and γδ T cells, particularly for use in adoptive immunotherapy. The invention also provides method for preparing such compositions which comprises contacting a sample with an anti-TCR delta variable 1 (anti-Vδ1) antibody.

### FIELD OF THE INVENTION

The invention relates to compositions comprising Natural Killer (NK) cells and γδ T cells, particularly for use in adoptive immunotherapy. The invention also provides method for preparing such compositions which comprises contacting a sample with an anti-TCR delta variable 1 (anti-Vδ1) antibody.

### BACKGROUND OF THE INVENTION

The recent success of non-cellular (e.g. immune checkpoint inhibitors) and cellular-immunotherapies (e.g. CD19 targeting αβ CAR-T cells) has prompted the development of novel immunotherapy approaches utilising unmodified and gene-engineered Tregs, iNK-T, γδ T-cells, NK cells and macrophages. Manufacturing of such advanced therapy medicinal products (ATMPs) requires a complex process utilising a combination of various cytokines, culture medium, stimulatory-beads and feeder cells. Widespread application of these therapies is currently cumbersome and expensive due to the complex manufacturing process and the underlying logistical challenges.

For example, the vast majority of CAR-modified αβ T-cell therapies are restricted to an autologous setting and the success of manufacturing runs are heavily pre-determined by the quality of the donor material. There are several drawbacks associated with these therapies. Firstly, αβ T cells are MHC-restricted resulting in the development of fatal graft versus host disease in an allogenic setting. Secondly, the majority of CAR-T therapies have been associated with unwanted side effects such as cytokine release syndrome, neurotoxicity, on-target off-tumour toxicities. Thirdly, the poor migration of αβ T cells to solid tumour sites hinders the therapeutic efficacy of solid tumour targeting approaches.

In order to aid the use of this ground-breaking technology in an allogenic setting the scientific field is pursuing two main strategies: (1) rendering T cells MHC unrestricted by using sophisticated gene editing methods (e.g. through knock out of native αβ TCRs) or (2) utilising inherently MHC unrestricted cell types (i.e. NK-cells and γδ T cells) as delivery vehicles to generate a truly allogenic cellular product. Both approaches come with their own challenges: gene editing makes the manufacturing process and quality control steps challenging, whereas the culture of unrestrictive cell types requires a well-defined cytokine milieu and/or feeder cell systems. Whilst there are established methods to grow NK cells either from blood sources or cell lines, most of these methods include the use of allogeneic feeder cells which need to be irradiated and removed afterwards. Most methods even go as far as using irradiated tumour cell lines (K562) that use genetically introduced membrane bound cytokines and co-stimulatory molecules as feeder layers during manufacturing. In some cases (i.e. NK-cell therapies), the widespread, off-the shelf application of the therapy is hindered by the poor cryopreservation and subsequent reduced cytotoxic potential of the cellular product.

Expansion methods for γδ T cells have previously been described using the addition of exogenous cytokines, for example see WO2017/072367 and WO2018/212808. Methods for expanding a patients' own γδ T cells has also been described using pharmacologically modified forms of hydroxy-methyl but-2-enyl pyrophosphate (HMBPP) or clinically-approved aminobisphosphonates. By these approaches, over 250 cancer patients have been treated, seemingly safely, but with only rare incidences of complete remission. There is still a need for methods that are able to expand large numbers of γδ T cells.

NK cells and γδ T cells are particularly attractive immunotherapeutic agents as they are MHC unrestricted and their activation does not depend on MHC bound antigen recognition. Even though they share some specific features (e.g. the ability to discriminate between healthy and malignant cells, the presence of natural cytotoxic receptors, etc.), they are also evolutionary conserved, and each cell type is equipped with unique immunological properties. This includes, but is not limited to, each cell type's unique mechanism of action such as the concept of missing self and NCR ligand recognition through NK cells as well as their potential to cooperate with antibodies to execute antibody mediated cellular cytotoxicity (ADCC). In addition to NCRs, Vδ1 cells also use an MHC unrestricted TCR to recognise tumour associated antigens and to migrate to specific tissue sites. Vδ2 T cells have a unique invariant TCR that allows them to recognise malignant cells by sensing increased metabolic activity of the mevalonate pathway, a feature that is linked to malignant cells with a mutated p53 oncogene. Additional to the broad spectrum of target recognition and modes of cytotoxicity, innate lymphocytes also bridge inflammation to sustained adaptive responses. For example, γδ T cells can interact with B cells and promote immunoglobulin (IG) class switching resulting in improved antibody responses to tumour antigen. Both, NK and γδ T cells attract and mature antigen presenting cells thus officially bridging the donor cells to the patient's own immune system with the potential to initiate long lasting immunological responses. The inflammatory milieu created by NK cells and γδ T cells furthermore attracts and actives conventional as T cells.

Whilst NK cells are very potent to fight against infectious disease (e.g. caused by viruses, bacteria or fungi), Vδ1 γδ T cells are fundamental for the recognition and protection against CMV reactivation (a significant problem in immune compromised patients, for example transplant recipients and heavily treated cancer patients). Furthermore, CMV activated Vδ1 γδ T cells significantly reduce the risk of secondary malignancies in immunosuppressant treated patients following transplantation.

Vδ2 γδ T cells are fundamentally important in recognising and eliminating mycobacterial infections through the recognition of HMBPP (a very high affinity intermediate of the non-mevalonate pathway mainly used by prokaryotes and some eukaryote cell types). Vδ2 cells also recognise protozoa and contribute greatly to the immune response against malaria infections.

Ideally, an allogenic off-the shelf cellular therapy product would combine all advantages that each of the above cell type uniquely owns: enhanced cytotoxicity against malignant cells, broad protection against viral and bacterial infections, etc.; and at the same time mitigates limitations of each singular cell product reducing the risk of antigen drift, tumour escape or immune suppression. Currently such combinational therapy is unavailable and would require separate manufacturing runs for each cell types followed by mixing of the cells at a pre-defined ratio. This invention aims to overcome this major manufacturing challenge.

### SUMMARY OF THE INVENTION

According to a first aspect of the invention, there is provided an isolated composition comprising Natural Killer (NK) cells and γδ T cells wherein at least 40% of the γδ T cells present in the composition are CD56^{bight}.

According to a further aspect of the invention, there is provided an isolated composition comprising NK cells and γδ T cells wherein at least 50% of the γδ T cells present in the composition express CD56.

According to a further aspect of the invention, there is provided an isolated composition comprising cells wherein at least 90% of the cells consist of NK cells and γδ T cells, wherein at least 10% of the cells are Vδ1 T cells, at least 5% of the cells are Vδ2 T cells and at least 30% of the cells are NK cells.

According to a further aspect of the invention, there is provided a method of expanding non-γδ+ MHC unrestricted lymphocytes comprising stimulating a mixed cell population comprising γδ T cells and NK cells using an anti-TCR delta variable 1 (anti-Vδ1) antibody or fragment thereof, in the presence of Interleukin-15 (IL-15) and in the absence of Interleukin-4 (IL-4) and culturing the mixed cell population.

According to a further aspect of the invention, there is provided a method of preparing a composition comprising a cell population enriched for MHC unrestricted lymphocytes, wherein the method comprises:
(1) culturing a sample obtained from a subject in the presence of:
   (i) an anti-Vδ1 antibody or fragment thereof; and
   (ii) IL-15, in the absence of IL-4,
      from the first day of said culturing; and
(2) isolating the cell population cultured from the sample.

According to a further aspect of the invention, there is provided a composition obtainable by a method as defined herein. According to a further aspect of the invention, there is provided a composition obtained by a method as defined herein.

According to a further aspect of the invention, there is provided a composition as defined herein for use in therapy.

According to a further aspect of the invention, there is provided a composition of as defined herein for use in a method of treating cancer, an infectious disease or an inflammatory disease.

According to a further aspect of the invention, there is provided a method of treating a cancer, an infectious disease or an inflammatory disease in a subject in need thereof, comprising administering a therapeutically effective amount of a composition comprising NK cells and γδ T cells wherein at least 40% of the γδ T cells present in the composition express CD56.

According to a further aspect of the invention, there is provided a method of treating a cancer, an infectious disease or an inflammatory disease in a subject in need thereof, comprising administering a therapeutically effective amount of a composition comprising cells wherein at least 90% of the cells consist of NK cells and γδ T cells, wherein at least 10% of the cells are Vδ1 T cells, at least 5% of the cells are Vδ2 T cells and at least 30% of the cells are NK cells.

### BRIEF DESCRIPTION OF THE FIGURES

FIG. 1: A schematic representation of condition 1, 2 and 3 cell culture regimes. In condition 1 cells are seeded and cultured with an anti-Vδ1 antibody, IL-4, IL-1β, IL-21 and IFNγ. Condition 1 cultures are further regularly fed with the anti-Vδ1 antibody, IL-21 and IL-15 up until harvest. In condition 2 cells are seeded and cultured with the anti-Vδ1 antibody and IL-15. Condition 2 cultures are further regularly fed with the anti-Vδ1 antibody and IL-15 up until harvest. In condition 3 cells are seeded and cultured with the anti-Vδ1 antibody, IL-15, IL-4, IL-1β, IL-21 and IFNγ. Condition 3 cultures are further regularly fed with α-Vδ1 antibody, IL-21 and IL-15 up until harvest. Conditions 1, 2 and 3 cultures are terminated and cells are harvested between days 11-14.

FIG. 2: Graph showing the total yield of cells harvested when cultured with conditions 1, 2 or 3. Cells are seeded and cultured in 24-well GREX plates. Upon harvest, cells were concentrated and counted using NucleoCounter NC250. Conditions 1 and 2 generate equivalent cell yields, while condition 3 increases cell yields relative to condition 1.

FIG. 3: Graph showing the immune cell composition of harvested cells when cultured under the different culture regimes (conditions 1, 2 or 3). Specifically, this figure shows the percentage of Vδ1+γδ T cells, Vδ2+γδ T cells and NK (CD3−CD56+) cells of total CD45+ lymphocytes. The percentage of distinct cell populations was assessed by flow cytometry. Conditions 1 and 3 generate a cell product highly enriched for Vδ1+ T cells, whereas condition 2 generated a cell product comprising a mixed population of Vδ1+T, Vδ2+T and NK cells.

FIG. 4: Graph showing the cell surface marker expression of key phenotypical markers in V151+γδ T cells cultured under the different culture regimes (condition 1, 2 or 3). More specifically, NKp30, CD27 and NKG2D cell surface expression was studied within Vδ1+γδ T cells. Phenotypical surface characterization was assessed by flow cytometry. Vδ1+ T cells generated from conditions 1 and 3 are highly positive for CD27 and NKG2D and low for NKp30. Vδ1+ T cells generated from conditions 2 express less CD27 but the expression of NKp30 and NKG2D is enhanced.

FIG. 5: Immune cell composition of cells harvested when seeded and cultured with different anti-Vδ1 antibodies or anti-CD3 antibody and IL-15. Specifically, this figure shows the percentage of Vδ1+γδ T cells, Vδ2+γδ T cells and NK (CD3−CD56+) cells of total CD45+ lymphocytes. The percentage of distinct cell populations was assessed by flow cytometry. The use of two separate anti-Vδ1 antibody clones enriches for NK cells as well as γδ T cells. The use of an anti-CD3 antibody only enriches for γδ T cells.

FIG. 6: Cell cytotoxicity. (A) shows the percentage of NALM-6 ALL tumor cell line killing upon co-culture with harvested cells cultured under the different culture regimes (condition 1, 2 or 3). Percentage of killing was assessed by flow cytometry after 20-22 hours of co-culture. (B) shows the percentage of CD56 positive lymphocytes and CD56 positive γδ T cells of total CD45+ lymphocytes. The percentage of distinct cell populations was assessed by flow cytometry. Condition 2 and 3 both markedly augment cytotoxicity activity towards NALM-6 tumor cells. The enhanced killing correlates with CD56 expression between the conditions.

FIG. 7: CD56 expression profile of Vδ1 and Vδ2 cells in PBMCs prior to culture, and following harvest from conditions 1 and 2. CD56 surface expression was determined through analysis of the staining intensity via flow cytometry. Condition 2 strongly enriches the intensity of CD56 expression on Vδ1 and Vδ2 cells, relative to their intrinsic PBMC level.

FIG. 8: CD56 expression on γδ T cells and NK cells In PBMCs prior to culture, and following harvest from condition 2. CD56 surface expression was determined by flow cytometry. Condition 2 strongly enriches the percentage of CD56-bright cells on both γδ T cells and NK cells.

FIG. 9: Viability and total cell recovery following cryopreservation of cells generated under the different culture regimes (condition 1 or 2). Total cell viability was assessed both using NucleoCounter NC250 and by flow cytometry. The percentage of total cell recovery is extrapolated from the total cell counted after thaw and wash of the cryopreserved cells relative to the amount of cells frozen down after culture termination and harvest. Equivalent viability and cell recovery is observed between conditions 1 and 2 following cryopreservation.

FIG. 10: Cellular phenotype post-cryopreservation. (A) shows representative flow plots of the immune cell composition of the cell product generated following condition 2 culture regime pre- and post-cryopreservation. (B) shows the phenotypical cell surface marker characterization of the three major populations (Vδ1+γδ T cells, Vδ2+γδ T cells and NK (CD3−CD56+) cells) present on the cell product generated following condition 2 culture regime pre- and post-cryopreservation. More specifically, NKp30, CD27, NKG2D and CD56 cell surface expression was analyzed in all immune cell populations present and compared to its expression pre- and post-cryopreservation. Condition 2 shows equivalent immune cell composition and cell surface marker phenotype pre- and post-cryopreservation.

FIG. 11: Cytotoxicity post-cryopreservation. The graph shows the percentage of NALM-6 ALL tumor cell line lysis upon co-culture with cell products generated from condition 1 and 2 culture regimes post-cryopreservation, at a wide range of effector to target ratios. Percentage of killing was assessed by flow cytometry after 20-22 hours of co-culture. Condition 2 shows enhanced cytotoxicity towards NALM-6 cells post-cryopreservation across a range of effector to target ratios relative to condition 1.

FIG. 12: Increased Permissiveness for Transduction. (A) shows a graph and (B) representative flow plots of the transduction with lentiviral vectors of all immune cells that compose the products generated from conditions 1 and 2. Transduction was performed in the presence of Retronectin. Transduced cell products were generated using crude cryopreserved material. Percentage of CAR19 positive cells is determined by flow cytometry 96 hours after transduction. Flow plots show representative plots of CAR expressing Vδ1+γδT cells. Condition 2 shows enhanced permissiveness to transduction relative to condition 1.

FIG. 13: Graph and representative flow plots of the enhanced CAR mediated cytotoxicity of the cells generated from condition 2 culture regime. More specifically, (A) shows the percentage of dead NALM-6 cells (Sytox+ve of CTV+ve) upon co-culture with CAR19 transduced and untransduced cells generated from condition 2 culture regime in a wide range of effector to target ratio. (B) shows representative flow plots of 1:50 effector to target ratio (top right) and apoptotic target cell discrimination (bottom right) of transduced cells generated from condition 2 upon co-culture with NALM-6. CAR19-transduced cells generated from condition 2 exhibit enhanced cytotoxicity towards NALM-6 cells. Cytotoxic targeting is observed at effector to target ratios as low as 1:100.

FIG. 14: Cell proportions and yields. (A) Schematic representation of culture regimen. Cells are seeded and cultured with an anti-Vδ1 antibody (clone C08) and IL-15. In this experimental set up cells were supplemented with IL-15 on days 6 and 9 until harvest on day 12. (B) Graph showing the immune cell composition (top) of harvested cells when cultured using the process described in (A) and the total yield and viability of harvested cells (bottom) from 5 donors.

FIG. 15: Cell proportions from multiple donors. (A) Graph showing the immune cell compositions of harvested cells when expanded as described in FIG. 14A, using 15 randomly chosen healthy donors. Specifically, this figure shows the percentage of Vδ1+γδ T cells, Vδ2+γδ T cells and NK (CD3−CD56+) cells of total CD45+ lymphocytes. (B) Table showing raw values.

FIG. 16: Relevance of CD56 expression to cytotoxicity. Induction of CD56 expression on innate immune cell combination (combo) consisting of γδ T cells and NK cells, expressed as (A) median fluorescence intensity (MFI), and (B) expressed as percentage of CD56+ on different immune cell subsets within combo between day 0 and 12 when cells are cultured as described in FIG. 14A. (C) Correlation between CD56 expression on CD3+ cells (γδ T cells) from 8 donors and cytotoxicity. Graph displays a linear regression between the percentage of sytox+ target cells and the percentage of CD56+ T-cells (R²=0.93, p=0.0001).

FIG. 17: Homing/Chemokine receptor profile of Immune cell combo. Cells were cultured as described in FIG. 14A and then cryopreserved. Upon thawing, cells were stained with antibodies for the displayed markers and acquired by flow cytometry. The graph shows the percentage of each immune subset expressing the marker of interest from 3 donors.

FIG. 18: Correlation between CD56 expression on Immune cell combo and transducibility. (A) Cells were cultured as shown in FIG. 14A and transduced as described in Example 8. The transduction efficiency on harvest is shown. The graph shows the total transduction efficiency as well as the transduction efficiency within each displayed member of the immune cell combo. (B) The transduction efficiency within the Vδ1+ subset stratified by CD56 expression. (C) Flow cytometry data (upper panel) showing CD56 expression on Vδ1+ cells cultured using C08 and IL-15 as shown in FIG. 14A or using OKT3+IL-4+IL-21+IFNγ+IL-1β (modified Condition 1 of Example 1). Graph (lower panel) shows the transduction efficiency within the Vδ1+ subset from the cells cultured using OKT3+IL-4+IL-21+IFNγ+IL-1β, stratified by CD56 expression. Lower CD56 expression is observed and the impact of CD56 expression on transduction efficiency in this condition is much lower compared to cells cultured using the process described in FIG. 14A.

FIG. 19: Cytotoxic targeting of solid tumour targets by Immune cell combination (‘combo’). Percentage of target cell lysis of (A) OVCAR cells (ovarian cancer origin) and (B) HeLa (cervical cancer origin) at indicated effector:target ratios using effector cells generated as described in FIG. 14A. Comparison of untransduced immune cell combo and gene engineered combo cells transduced with a mesothelin targeting chimeric antigen receptor including a 4-1BB and CD3 zeta intracellular signaling domain. As a control, state of the art αβ CAR-T cells using the same binder and signaling domains were used.

### DETAILED DESCRIPTION OF THE INVENTION

### Definitions

Unless defined otherwise, all technical and scientific terms used herein have the meaning commonly understood by a person skilled in the art to which this invention belongs. As used herein, the following terms have the meanings ascribed to them below.

Gamma delta (γδ) T cells represent a small subset of T cells that express on their surface a distinct, defining T Cell Receptor (TCR). This TCR is made up of one gamma (γ) and one delta (δ) chain. Each chain contains a variable (V) region, a constant (C) region, a transmembrane region and a cytoplasmic tail. The V region contains an antigen binding site. There are two major sub-types of human γδ T cells: one that is dominant in the peripheral blood and one that is dominant in non-haematopoietic tissues. The two sub-types may be defined by the type of δ and/or γ present on the cells. For example, γδ T cells that are dominant in peripheral blood primarily express the delta variable 2 chain (Vδ2). γδ T cells that are dominant in non-haematopoietic tissues (i.e. are tissue-resident) primarily express the delta variable 1 (Vδ1) chain. The main TGRV gene segments encoding Vγ are TRGV2, TRGV3, TRGV4, TRGV5, TRGV8, TRGV9 and TRGV11 and non-functional genes TRGV10, TRGV11, TRGVA and TRGVB. The most frequent TRDV gene segments encode Vδ1, Vδ2, and Vδ3, plus several V segments that have both Vδ and Vα designation (Adams et al., 296:30-40 (2015) Cell Immunol.).

References to “Vδ1 T cells” refer to γδ T cells with a Vδ1 chain, i.e. Vδ1⁺ T cells.

References to “delta variable 1” may also referred to as V1 or Vd1, while a nucleotide encoding a TCR chain containing this region may be referred to as “TRDV1”. Antibodies or fragments thereof which interact with the Vδ1 chain of a γδ TCR, are all effectively antibodies or fragments thereof which bind to Vδ1 and may referred to as “anti-TCR delta variable 1 antibodies or fragments thereof” or “anti-Vδ1 antibodies or fragments thereof”.

Additional references are made herein to other delta chains such as the “delta variable 2” chain. These can be referred to in a similar manner. For example, delta variable 2 chains can be referred to as Vδ2, while a nucleotide encoding a TCR chain containing this region may be referred to as “TRDV2”. In preferred embodiments antibodies or fragments thereof which interact with the Vδ1 chain of a γδ TCR, do not interact with other delta chains such as Vδ2

References to “gamma variable chains” are also made herein. These may also be referred to as γ-chains or Vγ, while a nucleotide encoding a TCR chain containing this region may be referred to as TRGV. For example, TRGV4 refers to Vγ4 chain. In a preferred embodiments, antibodies or fragments thereof which interact with the Vδ1 chain of a γδTCR, do not interact with gamma chains such as Vγ4.

References to “Natural Killer cell” or “NK cell” refer to a natural killer cells, an innate-like lymphocyte that does not express a TCR or CD3 and is mostly positive for expression of CD56. NK cells can also express the FC receptor CD16 and natural cytotoxicity receptors, such as NKp44 and NKp48 and NKG2D. Human NK cells can be divided into two subsets based on their cell-surface density of CD56-CD56^{bright} and CD56^{dim}—each with distinct phenotypic properties. Whereas most NK cells in peripheral blood are CD56^{dim}, CD56^{bright} NK cells are more abundant in tissues. CD56 expression is thought to be a marker for activation, with CD56^{dim} NK cells upregulating their CD56 expression to adopt a CD56^{bright} immunophenotype (Van Acker et al. (2017) Front. Immunol. 8: 892).

References to “MHC unrestricted lymphocytes” or “non-MHC restricted lymphocytes” refer to lymphocytes that do not require major histocompatibility complex (MHC) compatibility between the effector and target cell for recognition or to initiate an immune response. This term therefore refers to NK cells and γδ T cells because these lymphocytes do not require MHC recognition.

References to “non-γδ+ MHC unrestricted lymphocytes” refer to MHC unrestricted lymphocytes which do not express a γδ TCR (i.e. γδ−). Therefore, this term refers to NK cells.

The term “antibody” includes any antibody protein construct comprising at least one antibody variable domain comprising at least one antigen binding site (ABS). Antibodies include, but are not limited to, immunoglobulins of types IgA, IgG, IgE, IgD, IgM (as well as subtypes thereof). The overall structure of Immunoglobulin G (IgG) antibodies assembled from two identical heavy (H)-chain and two identical light (L)-chain polypeptides is well established and highly conserved in mammals (Padlan (1994) Mol. Immunol. 31:169-217).

A conventional antibody or immunoglobulin (Ig) is a protein comprising four polypeptide chains: two heavy (H) chains and two light (L) chains. Each chain is divided into a constant region and a variable domain. The heavy (H) chain variable domains are abbreviated herein as VH, and the light (L) chain variable domains are abbreviated herein as VL. These domains, domains related thereto and domains derived therefrom, may be referred to herein as immunoglobulin chain variable domains. The VH and VL domains (also referred to as VH and VL regions) can be further subdivided into regions, termed “complementarity determining regions” (“CDRs”), interspersed with regions that are more conserved, termed “framework regions” (“FRs”). The framework and complementarity determining regions have been precisely defined (Kabat et al. Sequences of Proteins of Immunological Interest, Fifth Edition U.S. Department of Health and Human Services, (1991) NIH Publication Number 91-3242). There are also alternative numbering conventions for CDR sequences, for example those set out in Chothia et al. (1989) Nature 342: 877-883. In a conventional antibody, each VH and VL is composed of three CDRs and four FRs, arranged from amino-terminus to carboxy-terminus in the following order FR1, CDR1, FR2, CDR2, FR3, CDR3, FR4. The conventional antibody tetramer of two heavy immunoglobulin chains and two light immunoglobulin chains is formed with the heavy and the light immunoglobulin chains inter-connected by e.g. disulphide bonds, and the heavy chains similarly connected. The heavy chain constant region includes three domains, CH1, CH2 and CH3. The light chain constant region is comprised of one domain, CL. The variable domain of the heavy chains and the variable domain of the light chains are binding domains that interact with an antigen. The constant regions of the antibodies typically mediate the binding of the antibody to host tissues or factors, including various cells of the immune system (e.g. effector cells) and the first component (C1q) of the classical complement system.

A fragment of the antibody (which may also referred to as “antibody fragment”, “immunoglobulin fragment”, “antigen-binding fragment” or “antigen-binding polypeptide”) as used herein refers to a portion of an antibody (or constructs that contain said portion) that specifically binds to the target, the delta variable 1 (Vδ1) chain of a γδ T cell receptor (e.g. a molecule in which one or more immunoglobulin chains is not full length, but which specifically binds to the target). Examples of binding fragments encompassed within the term antibody fragment include:
(i) a Fab fragment (a monovalent fragment consisting of the VL, VH, CL and CH1 domains);
(ii) a F(ab′)2 fragment (a bivalent fragment consisting of two Fab fragments linked by a disulphide bridge at the hinge region);
(iii) a Fd fragment (consisting of the VH and CH1 domains);
(iv) a Fv fragment (consisting of the VL and VH domains of a single arm of an antibody);
(v) a single chain variable fragment, scFv (consisting of VL and VH domains joined, using recombinant methods, by a synthetic linker that enables them to be made as a single protein chain in which the VL and VH regions pair to form monovalent molecules);
(vi) a VH (an immunoglobulin chain variable domain consisting of a VH domain);
(vii) a VL (an immunoglobulin chain variable domain consisting of a VL domain);
(viii) a domain antibody (dAb, consisting of either the VH or VL domain);
(ix) a minibody (consisting of a pair of scFv fragments which are linked via CH3 domains); and
(x) a diabody (consisting of a noncovalent dimer of scFv fragments that consist of a VH domain from one antibody connected by a small peptide linker a VL domain from another antibody).

“Human antibody” refers to antibodies having variable and constant regions derived from human germline immunoglobulin sequences. Human subjects administered with said human antibodies do not generate cross-species antibody responses (for example termed HAMA responses—human-anti-mouse antibody) to the primary amino acids contained within said antibodies. Said human antibodies may include amino acid residues not encoded by human germline immunoglobulin sequences (e.g. mutations introduced by random or site-specific mutagenesis or by somatic mutation), for example in the CDRs and in particular CDR3. However, the term is not intended to include antibodies in which CDR sequences derived from the germline of another mammalian species, such as a mouse, have been grafted onto human framework sequences. Human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g. a mouse) that is transgenic for human immunoglobulin genes or antibodies prepared, expressed, created or isolated by any other means that involves splicing of human immunoglobulin gene sequences to other DNA sequences, may also be referred to as “recombinant human antibodies”.

Substituting at least one amino acid residue in the framework region of a non-human immunoglobulin variable domain with the corresponding residue from a human variable domain is referred to as “humanisation”. Humanisation of a variable domain may reduce immunogenicity in humans.

“Specificity” refers to the number of different types of antigens or antigenic determinants to which a particular antibody or fragment thereof can bind. The specificity of an antibody is the ability of the antibody to recognise a particular antigen as a unique molecular entity and distinguish it from another. An antibody that “specifically binds” to an antigen or an epitope is a term well understood in the art. A molecule is said to exhibit “specific binding” if it reacts more frequently, more rapidly, with greater duration and/or with greater affinity with a particular target antigen or epitope, than it does with alternative targets. An antibody “specifically binds” to a target antigen or epitope if it binds with greater affinity, avidity, more readily, and/or with greater duration than it binds to other substances.

“Affinity”, represented by the equilibrium constant for the dissociation of an antigen with an antigen-binding polypeptide (KD), is a measure of the binding strength between an antigenic determinant and an antigen-binding site on the antibody (or fragment thereof): the lesser the value of the KD, the stronger the binding strength between an antigenic determinant and the antigen-binding polypeptide. Alternatively, the affinity can also be expressed as the affinity constant (KA), which is 1/KD. Affinity can be determined by known methods, depending on the specific antigen of interest.

Any KD value less than 10⁻⁶ is considered to indicate binding. Specific binding of an antibody, or fragment thereof, to an antigen or antigenic determinant can be determined in any suitable known manner, including, for example, Scatchard analysis and/or competitive binding assays, such as radioimmunoassays (RIA), enzyme immunoassays (EIA) and sandwich competition assays, equilibrium dialysis, equilibrium binding, gel filtration, ELISA, surface plasmon resonance, or spectroscopy (e.g. using a fluorescence assay) and the different variants thereof known in the art.

“Avidity” is the measure of the strength of binding between an antibody, or fragment thereof, and the pertinent antigen. Avidity is related to both the affinity between an antigenic determinant and its antigen binding site on the antibody and the number of pertinent binding sites present on the antibody.

“Human tissue Vδ1+ cells,” and “haemopoietic and blood Vδ1+ cells” and “tumour infiltrating lymphocyte (TIL) Vδ1+ cells,” are defined as cells expressing Vδ1 (Vδ1+) contained in or derived from either human tissue or the haemopoietic blood system or human tumours respectively. All said cell types can be identified by their (i) location or from where they are derived and (ii) their expression of the Vδ1+ TCR.

“Modulating antibodies” are antibodies that confer a measurable change including, but not limited to, a measurable change in cell cycle, and/or in cell number, and/or cell viability, and/or in one or more cell surface markers, and/or in the secretion of one or more secretory molecules (e.g., cytokines, chemokines, leukotrienes, etc.), and/or a function (such as cytotoxicity towards a target cell or diseased cell), upon contacting or binding to a cell expressing the target to which the antibody binds.

A method of “modulating” a cell, or population thereof, refers to a method wherein in at least one measurable change in said cell or cells, or secretion therefrom, is triggered to generate one or more “modulated cells”.

An “immune response” is a measurable change in at least one cell, or one cell-type, or one endocrine pathway, or one exocrine pathway, of the immune system (including but not limited to a cell-mediated response, a humoral response, a cytokine response, a chemokine response), such as upon addition of a modulating antibody.

An “immune cell” is defined as a cell of the immune system including, but not limited to, CD34+ cells, B-Cells, CD45+(lymphocyte common antigen) cells, Alpha-Beta T-cells, Cytotoxic T-cells, Helper T-cells, Plasma Cells, Neutrophils, Monocytes, Macrophages, Red Blood Cells, Platelets, Dendritic Cells, Phagocytes, Granulocytes, Innate lymphoid cells, Natural Killer (NK) cells and Gamma Delta T-cells. Typically, immune cells are classified with the aid of combinatorial cell surface molecule analysis (e.g., via flow cytometry) to identify or group or cluster to differentiate immune cells into sub-populations. These can be then still further sub-divided with additional analysis. For example, CD45+ lymphocytes can further sub-divided into vδ positive populations and vδ negative populations.

“Diseased cells” exhibit a phenotype associated with the progression of a disease such as a cancer, an infection such as a viral infection, or an inflammatory condition or inflammatory disease. For example, a diseased cell may be a tumour cell, an autoimmune tissue cell or a virally infected cell. Accordingly said diseased cells may be defined as tumorous, or virally infected, or inflammatory.

“Healthy cells” refers to normal cells that are not diseased. They may also be referred to as “normal” or “non-diseased” cells. Non-diseased cells include non-cancerous, or non-infected, or non-inflammatory cells. Said cells are often employed alongside relevant diseased cells to determine the diseased cell specificity conferred by a medicament and/or better understand the therapeutic index of a medicament

“Diseased-cell-specificity” is a measure of how effective an effector cell or population thereof, (such as, for example, a population of Vδ1+ cells) is at distinguishing and killing diseased cells, such as cancer cells, whilst sparing non-diseased or healthy cells. This potential can be measured in model systems and may involve comparing the propensity of an effector cell, or a population of effector cells, to selectively kill or lyse diseased cells versus the potential of said effector cell/s to kill or lyse non-diseased or healthy cells. Said diseased-cell-specificity can inform the potential therapeutic index of a medicament.

“Enhanced diseased-cell specificity” describes a phenotype of an effector cell such as, for example, a Vδ1+ cell, or population thereof, which has been modulated to further increase its capacity to specifically kill diseased cells. This enhancement can be measured in a variety of ways inclusive of fold-change, or percentage increase, in diseased-cell killing specificity or selectivity.

Suitably, the antibody or fragment thereof (i.e. polypeptide) of the invention is isolated. An “isolated” polypeptide or composition is one that is removed from its original environment. The term “isolated” may be used to refer to an antibody that is substantially free of other antibodies having different antigenic specificities (e.g. an isolated antibody that specifically binds Vδ1, or a fragment thereof, is substantially free of antibodies that specifically bind antigens other than Vδ1).

Suitably, the compositions used in the present invention are isolated. An “isolated” composition is one that is removed from its original environment For example, a naturally-occurring composition is isolated if it is separated from some or all of the coexisting materials in the natural system. A composition is considered to be isolated if, for example, it is prepared in vitro or ex vivo.

The antibody or fragment thereof may be a “functionally active variant” which also includes naturally occurring allelic variants, as well as mutants or any other non-naturally occurring variants. As is known in the art, an allelic variant is an alternate form of a (poly)peptide that is characterized as having a substitution, deletion, or addition of one or more amino acids that does essentially not alter the biological function of the polypeptide. By way of non-limiting example, said functionally active variants may still function when the frameworks containing the CDRs are modified, when the CDRs themselves are modified, when said CDRs are grafted to alternate frameworks, or when N- or C-terminal extensions are incorporated. Further, CDR containing binding domains may be paired with differing partner chains such as those shared with another antibody. Upon sharing with so called ‘common’ light or ‘common’ heavy chains, said binding domains may still function. Further, said binding domains may function when multimerized. Further, ‘antibodies or fragments thereof’ may also comprise functional variants wherein the VH or VL or constant domains have been modified away or towards a different canonical sequence (for example as listed at IMGT.org) and which still function.

For the purposes of comparing two closely-related polypeptide sequences, the “% sequence identity” between a first polypeptide sequence and a second polypeptide sequence may be calculated using NCBI BLAST v2.0, using standard settings for polypeptide sequences (BLASTP). For the purposes of comparing two closely-related polynucleotide sequences, the “% sequence identity” between a first nucleotide sequence and a second nucleotide sequence may be calculated using NCBI BLAST v2.0, using standard settings for nucleotide sequences (BLASTN).

Polypeptide or polynucleotide sequences are said to be the same as or “identical” to other polypeptide or polynucleotide sequences, if they share 100% sequence identity over their entire length. Residues in sequences are numbered from left to right, i.e. from N- to C-terminus for polypeptides; from 5′ to 3′ terminus for polynucleotides.

A “difference” between sequences refers to an insertion, deletion or substitution of a single amino acid residue in a position of the second sequence, compared to the first sequence. Two polypeptide sequences can contain one, two or more such amino acid differences. Insertions, deletions or substitutions in a second sequence which is otherwise identical (100% sequence identity) to a first sequence result in reduced % sequence identity. For example, if the identical sequences are 9 amino acid residues long, one substitution in the second sequence results in a sequence identity of 88.9%. If first and second polypeptide sequences are 9 amino acid residues long and share 6 identical residues, the first and second polypeptide sequences share greater than 68% identity (the first and second polypeptide sequences share 66.7% identity).

Alternatively, for the purposes of comparing a first, reference polypeptide sequence to a second, comparison polypeptide sequence, the number of additions, substitutions and/or deletions made to the first sequence to produce the second sequence may be ascertained. An “addition” is the addition of one amino acid residue into the sequence of the first polypeptide (including addition at either terminus of the first polypeptide). A “substitution” is the substitution of one amino acid residue in the sequence of the first polypeptide with one different amino acid residue. Said substitution may be conservative or non-conservative. A “deletion” is the deletion of one amino acid residue from the sequence of the first polypeptide (including deletion at either terminus of the first polypeptide).

A “conservative” amino acid substitution is an amino acid substitution in which an amino acid residue is replaced with another amino acid residue of similar chemical structure and which is expected to have little influence on the function, activity or other biological properties of the polypeptide. Such conservative substitutions suitably are substitutions in which one amino acid within the following groups is substituted by another amino acid residue from within the same group:

| Group | Amino acid residue |
|---|---|
| Non-polar aliphatic | Glycine |
| | Alanine |
| | Valine |
| | Methionine |
| | Leucine |
| | Isoleucine |
| Aromatic | Phenylalanine |
| | Tyrosine |
| | Tryptophan |
| Polar uncharged | Serine |
| | Threonine |
| | Cysteine |
| | Proline |
| | Asparagine |
| | Glutamine |
| Negatively charged | Aspartate |
| | Glutamate |
| Positively charged | Lysine |
| | Arginine |
| | Histidine |

Suitably, a hydrophobic amino acid residue is a non-polar amino acid. More suitably, a hydrophobic amino acid residue is selected from V, I, L, M, F, W or C.

As used herein, numbering of polypeptide sequences and definitions of CDRs and FRs are as defined according to the Kabat system (Kabat et al., 1991, herein incorporated by reference in its entirety). A “corresponding” amino acid residue between a first and second polypeptide sequence is an amino acid residue in a first sequence which shares the same position according to the Kabat system with an amino acid residue in a second sequence, whilst the amino acid residue in the second sequence may differ in identity from the first. Suitably corresponding residues will share the same number (and letter) if the framework and CDRs are the same length according to Kabat definition. Alignment can be achieved manually or by using, for example, a known computer algorithm for sequence alignment such as NCBI BLAST v2.0 (BLASTP or BLASTN) using standard settings.

References herein to an “epitope” refer to the portion of the target which is specifically bound by the antibody or fragment thereof. Epitopes may also be referred to as “antigenic determinants”. An antibody binds “essentially the same epitope” as another antibody when they both recognize identical or sterically overlapping epitopes. Commonly used methods to determine whether two antibodies bind to identical or overlapping epitopes are competition assays, which can be configured in a number of different formats (e.g. well plates using radioactive or enzyme labels, or flow cytometry on antigen-expressing cells) using either labelled antigen or labelled antibody.

Epitopes found on protein targets may be defined as “linear epitopes” or “conformational epitopes”. Linear epitopes are formed by a continuous sequence of amino acids in a protein antigen. Conformational epitopes are formed of amino acids that are discontinuous in the protein sequence, but which are brought together upon folding of the protein into its three-dimensional structure.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian and yeast vectors). Other vectors (e.g. non-episomal mammalian vectors) can be integrated into the genome of a host cell upon introduction into the host cell, and thereby are replicated along with the host genome.

Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g. replication defective retroviruses, adenoviruses and adeno-associated viruses), which serve equivalent functions, and also bacteriophage and phagemid systems. The term “recombinant host cell” (or simply “host cell”), as used herein, is intended to refer to a cell into which a recombinant expression vector has been introduced. Such terms are intended to refer not only to the particular subject cell but to the progeny of such a cell, for example, when said progeny are employed to make a cell line or cell bank which is then optionally stored, provided, sold, transferred, or employed to manufacture an antibody or fragment thereof as described herein.

References to “subject”, “patient” or “individual” refer to a subject, in particular a mammalian subject, to be treated. Mammalian subjects include humans, non-human primates, farm animals (such as cows), sports animals, or pet animals, such as dogs, cats, guinea pigs, rabbits, rats or mice. In some embodiment, the subject is a human. In alternative embodiments, the subject is a non-human mammal, such as a mouse.

The term “sufficient amount” means an amount sufficient to produce a desired effect. The term “therapeutically effective amount” is an amount that is effective to ameliorate a symptom of a disease or disorder. A therapeutically effective amount can be a “prophylactically effective amount” as prophylaxis can be considered therapy.

As used herein, the term “about” when used herein includes up to and including 10% greater and up to and including 10% lower than the value specified, suitably up to and inducing 5% greater and up to and including 5% lower than the value specified, especially the value specified. The term “between”, includes the values of the specified boundaries.

A disease or disorder is “ameliorated” if the severity of a sign or symptom of the disease or disorder, the frequency with which such a sign or symptom is experienced by a subject, or both, is reduced.

As used herein, “treating a disease or disorder” means reducing the frequency and/or severity of at least one sign or symptom of the disease or disorder experienced by a subject.

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

### Compositions

Compositions are provided herein that comprise a particularly advantageous “trinity” of cell types: NK cells, Vδ1 cells and Vδ2 cells. Both NK cells and γδ T cells lack MHC restriction and do not require MHC for activation. Although these cells share some biology allowing them to recognise tumour cells whilst sparing healthy cells, NK cells and γδ T cells, including subsets thereof, feature evolutionary conserved and unique properties only present in one specific cell type but not found across all innate lymphocytes. Therefore, a desirable cellular approach would combine the advantages of each cell type, supplementing a patient with an almost complete and highly primed innate immune system that can interact with the patient's own adaptive immune system, and orchestrate a bigger and sustained immunological response.

According to an aspect of the invention, there is provided a composition comprising NK cells and γδ T cells wherein at least 40% of the γδ T cells present in the composition are CD56^{bright}.

According to another aspect of the invention, there is provided a composition comprising NK cells and γδ T cells wherein at least 50% of the NK cells present in the composition are CD56^{bright}.

According to an aspect of the invention, there is provided a composition comprising NK cells and γδ T cells wherein at least 50% of the γδ T cells present in the composition express CD56.

CD56, also known as Neural Cell Adhesion Molecule (NCAM), is an adhesion molecule of the immunoglobulin (Ig) superfamily that correlates with high cytotoxicity in NK cells, αβ T cells and γδ T cells. It has been shown to participate in cis and trans binding to itself which contributes to lymphocyte activation. It has also been shown to be fundamental for immunological synapse formation between lymphocytes, lymphocytes and antigen presenting cells (APCs), as well as lymphocytes and target cells (Nussbaumer and Thumher (2020) Cells 9(3): 772).

Cell phenotype can also be defined by the cell-surface density of CD56. There are therefore recognised sub-types known in the art of CD56^{bright} and CD56^{dim} cells. The increased expression and/or intensity of CD56 on γδ T cells present in the compositions of the present invention are shown herein to have a correlation with enhanced killing, indicating the therapeutic potential of the compositions described herein.

In some embodiments at least about 40% of the γδT cells present in the composition are CD56^{bright}. In some embodiments, at least 45%, such as at least 46%, 47%, 48%, 49%, 50%, 51%, 52%, 53%, 54%, 55%, 56%, 57%, 58%, 59%, 60% of the γδ T cells present in the composition are CD56^{bright}. In some embodiments, at least about 50% of the γδ T cells present in the composition are CD56^{bright}. In some embodiments, at least about 55% of the γδ T cells present in the composition are CD56^{bright}. In some embodiments, at least about 60% of the γδ T cells present in the composition are CD56^{bright}.

In some embodiments at least about 50% of the NK cells present in the composition are CD56^{bright}. In some embodiments, at least 60%, such as at least 61%, 62%, 63%, 64%, 65%, 66%, 67%, 68%, 69%, 70%, 71%, 72%, 73%, 74%, 75%, 76%, 77%, 78%, 79%, 80% of the NK cells present in the composition are CD56^{bright}. In some embodiments, at least about 70% of the NK cells present in the composition are CD56^{bright}. In some embodiments, at least about 75% of the NK cells present in the composition are CD56^{bright}. In some embodiments, at least about 80% of the NK cells present in the composition are CD56^{bright}.

CD56 surface expression can be determined using methods known in the art, such as through analysis of the staining intensity via flow cytometry. Dividing cells into CD56^{bright} and CD56^{dim} is understood in the art, for example as described in Van Acker et at. (2017) Front. Immunol. 8: 892. Such methods compare the sample population to a reference population of known CD56 expression levels, such as a cell population containing NK cells. NK cells have distinct CD56 expression levels that can be identified as bright and dim, so by establishing gating strategies using this reference population, the sample population can also be sorted into CD56^{bright} and CD56^{dim} fractions for the purposes of this invention.

In some embodiments at least about 40% of the γδ T cells present in the composition express CD56. In some embodiments, at least 45%, such as at least 46%, 47%, 48%, 49%, 50%, 51%, 52%, 53%, 54%, 55%, 56%, 57%, 58%, 59%, 60% of the γδ T cells present in the composition express CD56. In some embodiments, at least about 50% of the γδ T cells present in the composition express CD56. In some embodiments, at least about 55% of the γδ T cells present in the composition express CD56. In some embodiments, at least about 60% of the γδ T cells present in the composition express CD56. In other embodiments, at least 70% of the γδ T cells present in the composition express CD56, such as at least 75%. In one embodiment, at least 80%, such as at least 85% of the γδ T cells present in the composition express CD56. In a further embodiment, at least 80% of the γδT cells present in the composition express CD56 after a period of about 12 days (such as 12 days) of culture as described herein.

In some embodiments, at least 80%, such as 85%, 90%, 95%, 97%, 98%, 99%, 99.5%, of the cells present in the composition comprise NK cells and γδT cells. In one embodiment, at least 85% of the cells present in the composition comprise NK and γδ T cells. In a further embodiment, at least 90% of the cells present in the composition comprise NK cells and γδ T cells. In a yet further embodiment, at least 95% of the cells present in the composition comprise NK cells and γδ T cells.

In some embodiments, at least 80%, such as 85%, 90%, 95%, 97%, 98%, 99%, 99.5%, of the cells present in the composition consist of NK cells and γδ T cells. In one embodiment, at least 85% of the cells present in the composition consist of NK and γδ T cells. In a further embodiment, at least 90% of the cells present in the composition consist of NK cells and γδ T cells. In a yet further embodiment, at least 95% of the cells present in the composition consist of NK cells and γδ T cells.

In some embodiments, at least 30% of the cells present in the composition are γδ T cells. In some embodiments, the composition comprises at least about 30% γδ T cells, such as at least about 31%, 32%, 33%, 34%, 35%, 36%, 37%, 38%, 39%, 40%, 41%, 42%, 43%, 44%, 45%, 48%, 47%, 48%, 49% or 50% γδ T cells. In a further embodiment, the composition comprises at least about 40% γδ T cells, such as greater than about 45% γδ T cells. In other embodiments, at least 15%, such as at least 20%, of cells present in the composition are γδ T cells. In one embodiment, the composition comprises at least about 20% γδ T cells.

In some embodiments, less than 80% of the cells present in the composition are γδ T cells. In some embodiments, the composition comprises less than about 80% γδ T cells, such as less than about 79%, 78%, 77%, 76%, 75%, 74%, 73%, 72%, 71%, 70%, 60%, 68%, 57%, 66%, 65%, 64%, 63%, 62%, 61%, 60%, 59%, 58%, 57%, 56%, 55%, 54%, 53%, 52%, 51% or 50% γδ T cells. In a further embodiment, the composition comprises less than about 60% γδ T cells. In some embodiments, the composition comprises between about 30% to 80% γδ T cells, such as about 30% to 60% γδ T cells. In a further embodiment, the composition comprises about 50% γδ T cells.

In some embodiments, the composition comprises at least about 10% Vδ1 T cells, such as at least about 11%, 12%, 13%, 14%, 15%, 16%, 17%, 18%, 19%, 20%, 21%, 22%, 23%, 24%, 25%, 26%, 27%, 28%, 29% or 30% Vδ1 T cells. In a further embodiment, the composition comprises at least about 20% Vδ1 T cells. In some embodiments, the composition comprises between about 10% to 55% Vδ1 T cells, such as between about 10% to 30% Vδ1 T cells. In one embodiment, the composition comprises about 30% Vδ1 T cells.

In some embodiments, the composition comprises at least about 5% Vδ2 T cells, such as at least about 6%, 7%, 8%, 9%, 10%, 11%, 12%, 13%, 14%, 15%, 16%, 17%, 18%, 19% or 20% Vδ2 T cells. In a further embodiment, the composition comprises at least about 7% Vδ2 T cells. In some embodiments, the composition comprises between about 7% to 30% Vδ2 T cells. In one embodiment, the composition comprises between about 15% to 20% Vδ2 T cells.

In some embodiments, the γδ T cells comprise Vδ1 cells, Vδ2 cells, Vδ3 cells, Vδ5 cells, and Vδ8 cells. In some embodiments, the γδT cells comprise Vδ1 T cells and Vδ2 T cells. In some embodiments, at least 70%, such as at least 80%, 85%, 90%, 95%, 97%, 98% or 99%, in particular at least 90% of the γδ T cells consist of Vδ1 T cells and Vδ2 T cells. In some embodiments, the γδ T cells consist of Vδ1 T cells and Vδ2 T cells. In some embodiments, the γδ T cells comprise non-Vδ1N62 T cells.

In some embodiments, at least 30% of the cells present in the composition are NK cells. In some embodiments, the composition comprises at least about 30% NK cells, such as at least about 31%, 32%, 33%, 34%, 35%, 36%, 37%, 38%, 39%, 40%, 41%, 42%, 43%, 44%, 45%, 48%, 47%, 48%, 49% or 50% NK cells. In a further embodiment, the composition comprises at least about 40% NK cells. In some embodiments, the composition comprises less than about 80% NK cells, such as less than about 79%, 78%, 77%, 76%, 75%, 74%, 73%, 72%, 71%, 70%, 60%, 68%, 57%, 66%, 65%, 64%, 63%, 62%, 61%, 60%, 59%, 58%, 57%, 56%, 55%, 54%, 53%, 52%, 51% or 50% NK cells. In a further embodiment, the composition comprises less than about 60% NK cells. In some embodiments, the composition comprises between about 20% to 70% NK cells, such as about 30% to 60% NK cells. In other embodiments, the composition comprises between about 40% to 50% NK cells. In a further embodiment, the composition comprises about 50% NK cells. In other embodiments, at least about 10% of the cells present in the composition are NK cells. In some embodiments, the composition comprises at least about 10% NK cells, such as at least 11%, 12%, 13%, 14%, 15%, 16%, 17%, 18%, 19% or 20% NK cells. In one embodiment, the composition comprises between about 10% to 75% NK cells. In a further embodiment, the composition comprises at least about 20% NK cells.

In some embodiments, the composition comprises less than about 10% αβ T cells, such as less than about 5%, 4%, 3%, 2%, 1.5%, 1%, 0.5%, 0.2%, 0.1% or 0.05% αβ T cells.

In a further embodiment, the compositions comprises less than about 1% αβ T cells. T cells with αβ receptors are highly reactive, therefore suitable cell populations for administration to patients in the context of the present invention can only contain low levels of αβ T cells.

Compositions of the invention may also be defined by the proportion of MHC unrestricted lymphocytes present in the composition. Therefore, according to a further aspect of the invention, there is provided a composition comprising cells wherein at least 90% of the cells consist of NK cells and γδ T cells, wherein at least 10% of the cells are Vδ1 T cells, at least 5% of the cells are Vδ2 T cells and at least 30% of the cells are NK cells. The proportions of Vδ1, Vδ2 and NK cells may be as described hereinbefore. In some embodiments, at least 13% of the cells are Vδ1 T cells, at least 7% of the cells are Vδ2 T cells and at least 35% of the cells are NK cells. In some embodiments, at least 20% of the cells are Vδ1 T cells, at least 10% of the cells are Vδ2 T cells and at least 40% of the cells are NK cells. In other embodiments, at least 30% of the cells are Vδ1 T cells, at least 20% of the cells are Vδ2 T cells and at least 35% of the cells are NK cells.

As described herein, the γδ T cells may comprise Vδ1 T cells. In some embodiments, the Vδ1 T cells present in the composition expresses a high level of NKp30. For example, more than about 10%, such as more than about 15%, 20% or 25% of the Vδ1 T cells express NKp30 (i.e. NKp30+). In some embodiments, the γδ T cells comprise Vδ1 T cells and at least 15% of said Vδ1 T cells express NKp30.

In some embodiments, the Vδ1 T cells present in the composition express a low level of CD27 (CD27^{low}). For example, less than about 70%, such as less than about 50%, 40% or 30% of the Vδ1 T cells express CD27 (i.e. CD27+). In some embodiments, the γδ T cells comprise Vδ1 T cells and less than 50% of said Vδ1 T cells express CD27.

### Compositions Comprising Engineered Cells

Innate lymphocytes do not control proliferation, activation, cytokine production and killing through one receptor. Hence, the composition described herein would allow for genetic engineering strategies that are impossible or less advantageous in as T cells or even within one innate cell type specifically. For example, a nonsignaling CAR can be used, such as the nonsignaling CAR described in WO2019180279. The use of specific domains would initiate a balanced response, e.g. DAP10 could trigger responses like activation and killing in NK cells, and Th1 cytokine production in Vδ1 T cells.

In some embodiments, the isolated composition comprises engineered NK cells and γδ T cells. As shown in the examples provided herein, the cells of the claimed composition have been shown to have increased permissiveness for transduction making them suitable for genetic engineering methods.

The cells of the composition may be engineered to express one or more transgenes, which may encode, for example, a membrane-bound protein (e.g. a cell surface receptor, such as a chimeric antigen receptor (CAR), an αβ TCR, a natural cytotoxicity receptor (e.g. NKp30, NKp44, or NKp46), a cytokine, a cytokine receptor (e.g. IL-12 receptor), a chemokine receptor (e.g. CCR2 receptor)), a selectable marker (e.g. a reporter gene), or a suicide gene. In some instances, the invention provides a population of MHC unrestricted lymphocytes engineered to express a CAR and optionally one or more additional transgene-encoded proteins (e.g. an armor protein). In some embodiments, the one or more transgenes are codon-optimized.

In some embodiments, the engineered NK cells and γδ T cells express a chimeric antigen receptor (CAR). The term “chimeric antigen receptor”, or alternatively a “CAR”, refers to a recombinant polypeptide construct including an extracellular antigen binding domain, a transmembrane domain, and an intracellular domain that propagates an activation signal that activates the cell. In some embodiments, the CAR includes an optional leader sequence at the N-terminus of the CAR fusion protein.

In some embodiments, at least 20% (e.g. at least 20%, 25%, 30%, 35%, 40%, 45%, 50%, 55%, 60%, 65%, 70%, 75%, 80%, 85%, 90%, 95%, 97%, 99%, or substantially all) of the engineered cells express the transgene, e.g. the CAR or other membrane-bound or soluble protein. In some embodiments, at least 50% (e.g. at least 50%, 55%, 60%, 65%, 70%, 75%, 80%, 85%, 90%, 95%, 97%, 99%, or substantially all) of the engineered cells express the transgene, e.g. the CAR or other membrane-bound or soluble protein. In some embodiments, 20%-80% of the engineered cells express the transgene, e.g. the CAR or other membrane-bound or soluble protein. In other embodiments, between about 20% to 60% of the engineered cells express the transgene, e.g. the CAR or other membrane-bound or soluble protein. In some embodiments, 40%-70% of the engineered cells express the transgene, e.g. the CAR or other membrane-bound or soluble protein.

In some embodiments, at least about 20% (e.g. at least 20%, 25%, 30%, 35% or 40%) of the engineered NK cells express the transgene, e.g. the CAR or other membrane-bound or soluble protein. In some embodiments, at least 40% (e.g. at least 40%, 45%, 50%, 55%, 60%, 65%, 70%, 75%, 80%, 85%, 90%, 95%, 97%, 99%, or substantially all) of the engineered NK cells express the transgene, e.g. the CAR or other membrane-bound or soluble protein. In some embodiments, at least 50% (e.g. at least 50%, 55%, 60%, 65%, 70%, 75%, 80%, 85%, 90%, 95%, 97%, 99%, or substantially all) of the engineered NK cells express the transgene, e.g. the CAR or other membrane-bound or soluble protein. In other embodiments, at least 70% (e.g. at least 70%, 75%, 80%, 85%, 90%, 95% or substantially all) of the engineered NK cells express the transgene, e.g. the CAR or other membrane-bound or soluble protein. In some embodiments, 40%-95% of the engineered NK cells express the transgene, e.g. the CAR or other membrane-bound or soluble protein. In some embodiments, 50%-80% of the engineered NK cells express the transgene, e.g. the CAR or other membrane-bound or soluble protein. In other embodiments, between about 20% to 70% of the engineered NK cells express the transgene, e.g. the CAR or other membrane-bound or soluble protein.

In some embodiments, at least 20% (e.g. at least 20%, 25%, 30%, 35%, 40%, 45%, 50%, 55%, 60%, 65%, 70%, 75%, 80%, 85%, 90%, 95%, 97%, 99%, or substantially all) of the engineered γδ T cells express the transgene, e.g. the CAR or other membrane-bound or soluble protein. In some embodiments, at least 30% (e.g. at least 30%, 35%, 40%, 45%, 50%, 55%, 60%, 65%, 70%, 75%, 80%, 85%, 90%, 95%, 97%, 99%, or substantially all) of the engineered γδ T cells express the transgene, e.g. the CAR or other membrane-bound or soluble protein. In some embodiments, at least about 50% (e.g. at least 50%, 55%, 60%, 65%, 70%, 75%, 80%, 85%, 90%, 95% or substantially all) of the engineered γδ T cells express the transgene, e.g. the CAR or other membrane-bound or soluble protein. In some embodiments, 20%-60% of the engineered γδT cells express the transgene, e.g. the CAR or other membrane-bound or soluble protein. In some embodiments, 30%-55% of the engineered γδ T cells express the transgene, e.g. the CAR or other membrane-bound or soluble protein. In other embodiments, between about 35% to 60% of the engineered γδ T cells express the transgene, e.g. the CAR or other membrane-bound or soluble protein.

As also shown in the examples provided herein, CD56+ cells of the claimed composition have been shown to have still increased permissiveness for transduction making them particularly suitable for genetic engineering methods.

In some embodiments, at least 20% (e.g. at least 20%, 25%, 30%, 35%, 40%, 45%, 50%, 55%, 60%, 65%, 70%, 75% or 80%) of the engineered CD56+ cells express the transgene, e.g. the CAR or other membrane-bound or soluble protein. In other embodiments, at least about 40% (e.g. at least 40%, 45%, 50%, 55%, 60%, 65%, 70%, 75% or 80%) of the engineered CD56+ cells express the transgene, e.g. the CAR or other membrane-bound or soluble protein. In some embodiments, between about 20% to 80% of the engineered CD56+ cells express the transgene, e.g. the CAR or other membrane-bound or soluble protein. In other embodiments, between about 45% to 75% of the engineered CD56+ cells express the transgene, e.g. the CAR or other membrane-bound or soluble protein.

### Pharmaceutical Compositions

According to a further aspect of the invention, there is provided a composition obtained by a method as defined herein. According to a further aspect of the invention, there is provided a pharmaceutical composition comprising the cell population obtained by a method as defined herein. In such embodiments, the composition may comprise the cells, optionally in combination with other excipients. Also included are compositions comprising one or more additional active agents (e.g. active agents suitable for treating the diseases mentioned herein).

Pharmaceutical compositions may include MHC unrestricted lymphocytes, in particular expanded MHC unrestricted lymphocytes as described herein, in combination with one or more pharmaceutically or physiologically acceptable carrier, diluents, or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g. aluminium hydroxide); and preservatives.

Cryopreservation solutions which may be used in the pharmaceutical compositions of the invention include, for example, DMSO. Compositions can be formulated, e.g. for intravenous administration.

In one embodiment, the pharmaceutical composition is substantially free of, e.g. there are no detectable levels of a contaminant, e.g. of endotoxin or mycoplasma.

The preferred mode of administration is parenteral (e.g., intravenous, subcutaneous, intraperitoneal, intramuscular, intrathecal). In a preferred embodiment, the composition is administered by intravenous infusion or injection. In another preferred embodiment, the composition is administered by intramuscular or subcutaneous injection.

It is within the scope of the invention to use the pharmaceutical composition of the invention in therapeutic methods for the treatment of diseases as described herein as an adjunct to, or in conjunction with, other established therapies normally used in the treatment of such diseases.

In a further aspect of the invention, the cell population, composition or pharmaceutical composition is administered sequentially, simultaneously or separately with at least one active agent.

### Methods of Preparing Compositions Enriched for MHC Unrestricted Lymphocytes

The invention provides methods for preparing compositions which are enriched for MHC unrestricted lymphocytes. Such methods are particularly advantageous for preparing compositions suitable for allogeneic use because the therapeutic effect will not be restricted by MHC compatibility.

According to an aspect of the invention, there is provided a method of expanding non-γδ+ MHC unrestricted lymphocytes comprising stimulating a mixed cell population comprising γδ T cells and NK cells using an anti-TCR delta variable 1 (anti-Vδ1) antibody or fragment thereof, in the presence of Interleukin-15 (IL-15) and in the absence of Interleukin-4 (IL-4) and culturing the mixed cell population. It will be understood that said mixed cell population may be obtained from a sample as described in more detail herein.

The inventors have surprisingly found that the presence of an anti-Vδ1 antibody and IL-15 stimulates the production of a composition which is a fully MHC unrestricted CD56+ immunotherapy. Without being bound by theory, the Vδ1 T cells appear to be stimulated by the presence of antibodies that specifically bind to the Vδ1 TCR and unexpectedly support non-Vδ1 cell expansion, for example NK cell expansion. Without being bound by the theory, this non-Vδ1 cell expansion, such as NK cell expansion, may be mediated via the anti-Vδ1 antibody and CD16 expressed on the NK cell surface. The unique cell composition produced by the method of the invention provides a mixture of cells with very promising therapeutic applications.

According to an aspect of the invention, there is provided a method of preparing a composition comprising a cell population enriched for MHC unrestricted lymphocytes, wherein the method comprises:
(1) culturing a sample obtained from a subject in the presence of:
   (i) an anti-TCR delta variable 1 (anti-Vδ1) antibody or fragment thereof; and
   (ii) Interleukin-15 (IL-15), in the absence of Interleukin-4 (IL-4), from the first day of said culturing; and
(2) isolating the cell population cultured from the sample.

It will be understood that the culturing step may be performed in vitro or ex vivo. The initial sample may comprise a mixed cell population, e.g. a cell population comprising more than one immune cell type, such as γδ T cells, NK cells and optionally αβ T cells, preferably γδ T cells and NK cells only. Therefore, in one embodiment, the sample comprises a mixed cell population which comprises γδ T cells and NK cells.

In a further embodiment, the sample is enriched for T cells prior to culturing, such as prior to administration of the anti-Vδ1 antibody or fragment thereof.

In some embodiments, the sample is depleted of cells types other than MHC unrestricted lymphocytes present in the sample, such as depleted of cells types other than NK cells and γδ T cells, prior to administration of the anti-Vδ1 antibody or fragment thereof. For example, prior to culturing the sample, the sample may be enriched for T cells and/or depleted of αβ T cells. In one embodiment, the sample is first depleted of αβ T cells. Enrichment or depletion may be achieved using techniques known in the art, such as using magnetic beads coated with antibodies that bind to molecules on the cell surface relevant to the phenotype to be enriched/depleted.

The presence of cell types other than lymphocytes in cell culture, may inhibit MHC unrestricted lymphocyte expansion. Such cells, e.g. stromal, epithelial, tumour and/or feeder cells, may be removed prior to culture. Thus, in one embodiment, the cell population is not in direct contact with stromal cells during culture. Examples of stromal cells include fibroblasts, pericytes, mesenchymal cells, keratinocytes, endothelial cells and non-haematological tumour cells. Preferably, the lymphocytes are not in direct contact with fibroblasts during culture. In one embodiment, the cell population is not in direct contact with epithelial cells during culture. In one embodiment, the cell population is not in direct contact with tumour cells and/or feeder cells during culture.

In one embodiment, the method comprises culturing the MHC unrestricted lymphocytes in the absence of substantial stromal cell contact. In a further embodiment, the method comprises culturing the MHC unrestricted lymphocytes in the absence of substantial fibroblast cell contact.

In one embodiment, the method comprises culturing the sample in media which contains plasma (e.g. human plasma). In a further embodiment, the sample is cultured in media comprising 2.5% plasma, such as 2.5% human plasma.

In one embodiment, the method comprises culturing the sample in media which is substantially free of serum (e.g. serum-free media or media containing a serum-replacement (SR)). Thus, in one embodiment, the method comprises culturing in serum-free media. Such serum free medium may also include serum replacement medium, where the serum replacement is based on chemically defined components to avoid the use of human or animal derived serum. In an alternative embodiment, the method comprises culturing in media which contains serum (e.g. human AB serum or fetal bovine serum (FBS)). In one embodiment, the media contains serum-replacement. In one embodiment, the media contains no animal-derived products.

It will be appreciated that a sample cultured in serum-free media has the advantage of avoiding issues with filtration, precipitation, contamination and supply of serum. Furthermore, animal derived products are not favoured for use in clinical grade manufacturing of human therapeutics. Use of serum-free media for the cells, particularly Vδ1 T cells, substantially increases the number of cells obtained from the sample compared to the use of media containing AB serum.

In one embodiment, the anti-Vδ1 antibody or fragment thereof is in a soluble or immobilized form. For example, the antibody or fragment thereof may be administered to the sample in a soluble form. Alternatively, the antibody or fragment thereof may be administered to the sample when the antibody or fragment thereof is bound or covalently linked to a surface, such as a bead or plate (i.e. in an immobilized form). In one embodiment, the antibody is immobilized on a surface, such as Fc-coated wells. Alternatively, the antibody or fragment thereof is bound to the surface of a cell (e.g. immobilized on the surface of an antigen presenting cell (APC)). In another embodiment, the antibody is not immobilized on a surface when the cell population is contacted with the antibody.

The cell population contacted by the anti-Vδ1 antibody or fragment thereof may be obtained from a variety of sample types. In one embodiment, the sample is a haematopoietic sample or fraction thereof (i.e. the cell population is obtained from a haematopoietic sample or a fraction thereof). References herein to “haematopoietic sample” or “haematopoietic tissue sample” include blood (such as peripheral blood or umbilical cord blood), bone marrow, lymphoid tissue, lymph node tissue, thymus tissue, and fractions or enriched portions thereof. The sample is preferably blood including peripheral blood or umbilical cord blood or fractions thereof, including buffy coat cells, leukapheresis products, peripheral blood mononuclear cells (PBMCs) and low density mononuclear cells (LDMCs). In some embodiments, the haematopoietic sample consists of low density mononuclear cells (LDMCs) or peripheral blood mononuclear cells (PBMCs). In some embodiments the sample is human blood or a fraction thereof. The cells may be obtained from a sample of blood using techniques known in the art such as density gradient centrifugation. For example, whole blood may be layered onto an equal volume of FICOLL-HYPAQUE followed by centrifugation at 400×g for 15-30 minutes at room temperature. The interface material will contain low density mononuclear cells which can be collected and washed in culture medium and centrifuged at 200×g for 10 minutes at room temperature. In some embodiments, the cell population is not obtained from particular types of samples, such as non-haematopoietic tissue samples, for example skin.

In an alternative embodiment, the sample is a non-haematopoietic tissue sample. References herein to “non-haematopoietic tissues” or “non-haematopoietic tissue sample” include skin (e.g. human skin) and gut (e.g. human gut). Non-haematopoietic tissue is a tissue other than blood, bone marrow, lymphoid tissue, lymph node tissue, or thymus tissue. In one embodiment, the non-haematopoietic tissue sample is skin (e.g. human skin). In some embodiments, the cell population is obtained from skin (e.g. human skin), which can be obtained by methods known in the art. For example, the cell population may be obtained from the non-haematopoietic tissue sample by culturing the non-haematopoietic tissue sample on a synthetic scaffold configured to facilitate cell egress from the non-haematopoietic tissue sample. Alternatively, the methods can be applied to a cell population (e.g. γδ T cells) obtained from the gastrointestinal tract (e.g. colon or gut), mammary gland, lung, prostate, liver, spleen, pancreas, uterus, vagina and other cutaneous, mucosal or serous membranes.

The sample may be obtained from a cancer tissue sample (i.e. the γδ T and NK cells may also be resident in cancer tissue samples), e.g. tumours of the breast or prostate. In some embodiments, the sample may be from human cancer tissue samples (e.g. solid tumour tissues). In other embodiments, the sample may be from a source other than human cancer tissue (e.g. a tissue without a substantial number of tumour cells). For example, the sample may be from a region of skin (e.g. healthy skin) separate from a nearby or adjacent cancer tissue. Thus, in some embodiments, the sample is not obtained from cancer tissue (e.g. human cancer tissue).

The sample may be obtained from human or non-human animal tissue. Therefore, the method may additionally comprise a step of obtaining a sample from human or non-human animal tissue. In one embodiment the sample has been obtained from a human. In an alternative embodiment, the sample has been obtained from a non-human animal subject.

In some embodiments, the cell population contacted by the anti-Vδ1 antibody or fragment thereof is cultured in a volume of at least about 10 mL, such as bout 15 mL, 20 mL, 30 mL, 40 mL, 50 mL, 60 mL, 70 mL, 80 mL, 90 mL, 100 mL or greater. In one embodiment, the cell population is cultured in about 30 mL. In another embodiment, the cell population is cultured in about 100 mL.

In some embodiments, the cell population contacted by the anti-Vδ1 antibody or fragment thereof is cultured at a cell density of at least about 0.5×10⁶ cells/cm², such as about 1×10⁶ cells/cm², 1.5×10⁶ cells/cm², 2×10⁶ cells/cm², 2.5×10⁶ cells/cm² or 3×10⁶ cells/cm². In one embodiment, the cell population is cultured at a cell density of about 1×10⁶ cells/cm². In another embodiment, the cell population is cultured at a cell density of about 2×10⁶ cells/cm².

The invention therefore provides ex vivo methods for producing an enriched MHC unrestricted lymphocyte population. The enriched population can be produced from an isolated mixed cell populations (e.g. obtained from samples taken from patients/donors) by a method comprising contacting the mixed cell population, or a purified fraction thereof, with the antibody or fragment thereof.

Thus, provided herein is a cell population enriched for MHC unrestricted lymphocytes obtainable, such as obtained, according to the method as defined herein. According to this aspect of the invention, it will be appreciated that such an expanded population of MHC unrestricted lymphocytes may be obtained and/or expanded in vitro or ex vivo. In one aspect, there is provided an expanded population of MHC unrestricted lymphocytes obtainable, such as obtained, according to the method as defined herein, wherein the population is isolated and expanded in vitro or ex vivo.

Also provided is an expanded NK cell population obtained according to the method as defined herein. According to this aspect of the invention, it will be appreciated that such an expanded population of NK cells may be obtained and/or expanded in vitro or ex vivo. In one aspect, there is provided an expanded NK cell population obtained according to the method as defined herein, wherein the NK cell population is isolated and expanded in vitro or ex vivo.

Also provided is an expanded γδ T cell population obtained according to the method as defined herein. According to this aspect of the invention, it will be appreciated that such an expanded population of γδ T cells may be obtained and/or expanded in vitro or ex vivo. In one aspect, there is provided an expanded γδ T cell population obtained according to the method as defined herein, wherein the γδ T cell population is isolated and expanded in vitro or ex vivo.

Antibodies or fragments thereof as described herein may be used in methods of expanding MHC unrestricted lymphocytes. These methods may be carried out in vitro or ex vivo. If the expansion methods are carried out in vitro, the antibodies (or fragments thereof) may be applied to an isolated cell population obtained as described above. In some embodiments, the cells are expanded from a cell population that has been isolated from a haematopoietic tissue sample, such as a blood sample.

Expansion of MHC unrestricted lymphocytes comprises culturing the sample in the presence of the antibody or fragment thereof as described herein, and interleukin-15 (IL-15).

Such culturing may occur in the presence or absence of other cytokines. Cytokines may include interleukins, lymphokines, interferons, colony stimulating factors and chemokines. The data presented herein shows that culturing the sample in the presence of IL-15 and absence of interleukin-4 (IL-4) from the start of culturing preferentially expands MHC unrestricted lymphocytes. Therefore, the methods described herein comprise culturing the sample in the presence of an anti-Vδ1 antibody (or fragment thereof) and IL-15, in the absence of IL-4, from the start (i.e. the first day) of culturing. It will be understood that references to the cytokines as described herein, may include any compound that has the same activity as said cytokine with respect to its ability to promote similar physiological effects on MHC unrestricted lymphocytes in culture and includes, but is not limited to, mimetics, or any functional equivalent thereof.

In some embodiments, the methods described herein comprise adding an anti-Vδ1 antibody (or fragment thereof) at the start (i.e. the first day) of culture. In further embodiments, the methods described herein comprise adding an anti-Vδ1 antibody (or fragment thereof) only at the start (i.e. only on the first day) of culture. In other embodiments, the methods described herein comprise adding anti-Vδ1 antibody at one or more additional points during the culture period (e.g. at one or two additional time points). In some embodiments, the methods described herein comprise adding IL-15 at the start (i.e. the first day) of culture. In further embodiments, the methods described herein comprise adding IL-15 only at the start (i.e. only on the first day) of culture. In other embodiments, the methods described herein comprise adding IL-15 at one or more additional points during the culture period (e.g. at one or two additional time points). As will be readily appreciated, reduced frequency of addition of anti-Vδ1 antibody and/or IL-15 provide the advantage of reduced manipulation of the cultures, e.g. reduced handling. Such reduced manipulation/handing may lead to improved manufacturability.

The culturing step may be performed in the presence or absence of other cytokines.

Examples of cytokines include, but are not limited to, interleukin-2 (IL-2), interleukin-6 (IL-6), interleukin-7 (IL-7), interleukin-8 (IL-8), interleukin-9 (IL-9), interleukin-12 (IL-12), interleukin-18 (IL-18), interleukin-21 (IL-21), interleukin-33 (IL-33), insulin-like growth factor 1 (IGF-1), interleukin-1β (IL-1β), interferon-γ (IFN-γ) and stromal cell-derived factor-1 (SDF-1).

The cytokine (e.g. an interleukin) used may be of human or animal origin, preferably of human origin. It may be a wild-type protein or any biologically active fragment or variant, that is, to say, capable of binding its receptor. Such binding may induce activation of γδ T cells in the conditions of a method according to the invention. More preferably, the cytokine may be in soluble form, fused or complexed with another molecule, such as for example a peptide, polypeptide or biologically active protein. Preferably, a human recombinant cytokine is used. More preferably, the range of interleukin concentration could vary between 1-10000 ng/ml, even more preferably between 1-1000 ng/ml, such as about 100 ng/ml. In some embodiments, the concentration of IL-15 is 100 ng/ml. Thus, in one embodiment the culturing step is performed in the presence of 100 ng/ml IL-15.

In one embodiment, the cytokine is a growth factor having interleukin-15-like activity, i.e. any compound that has the same activity as IL-15 with respect to its ability to promote similar physiological effects on MHC unrestricted lymphocytes in culture and includes, but is not limited to, IL-15 and IL-15 mimetics, or any functional equivalent of IL-15, including IL-2 and IL-7.

In one embodiment, the culture medium is in the presence of IL-15. In one embodiment, the culture medium is in the absence of IL-4.

As used herein, references to “expanded” or “expanded population” includes populations of cells which are larger or contain a larger number of cells than a non-expanded population. Such populations may be large in number, small in number or a mixed population with the expansion of a proportion or particular cell type within the population. It will be appreciated that the term “expansion method” refers to processes which result in expansion or an expanded population. Thus, expansion or an expanded population may be larger in number or contain a larger number of cells compared to a population which has not had an expansion step performed or prior to any expansion step. It will be further appreciated that any numbers indicated herein to indicate expansion (e.g. fold-increase or fold-expansion) are illustrative of an increase in the number or size of a population of cells or the number of cells and are indicative of the amount of expansion.

In one embodiment, the method comprises culturing the sample for at least 5 days (e.g. at least 6 days, at least 7 days, at least 8 days, at least 9 days, at least 10 days, at least 11 days, at least 12 days, at least 13 days, at least 14 days, at least 18 days, at least 21 days, at least 28 days, or longer, e.g. from 5 days to 40 days, from 7 days to 35 days, from 14 days to 28 days, or about 21 days). In a further embodiment, the method comprises culturing the sample for at least 7 days, such as at least 11 days or at least 14 days.

In further embodiments, method comprises culturing the sample for a duration (e.g. at least 5 days, at least 6 days, at least 7 days, at least 8 days, at least 9 days, at least 10 days, at least 11 days, at least 12 days, at least 13 days, at least 14 days, at least 18 days, at least 21 days, at least 28 days, or longer, e.g. from 5 days to 40 days, from 7 days to 35 days, from 14 days to 28 days, or about 21 days) in an amount effective to produce an expanded population of MHC unrestricted lymphocytes.

In one embodiment, the sample is cultured for a period of 5 to 60 days, such as at least 7 to 45 days, 7 to 21 days, or 7 to 18 days. In one embodiment, the sample is cultured for a period of 11 to 14 days. In a further embodiment, the sample is cultured for a period of 12 days to produce an expanded population of MHC unrestricted lymphocytes. In another embodiment, the sample is cultured for a period of 14 days. In a yet further embodiment, the sample is cultured for a period until expression of a marker of active proliferation, such as Ki-67, is downregulated, i.e. for a period before such downregulation may be detected. Thus, in one embodiment the sample is cultured for a period before expression of a marker of active proliferation, such as Ki-67, is downregulated. In certain embodiments, the culture period until/before expression of a maker of active proliferation is downregulated is 12 days. In one embodiment, the culture period until/before expression of Ki-67 is downregulated is 12 days.

The method may comprise regular addition of the anti-Vδ1 antibody or fragment thereof and/or growth factor during culturing. For example, the anti-Vδ1 antibody or fragment thereof and/or growth factor could be added every 2 to 7 days, more preferably every 3 to 4 days. In one embodiment, (after initial administration) the anti-Vδ1 antibody or fragment thereof and/or growth factor is added after 7 days of culture and every 3 to 4 days thereafter.

Methods of the invention provide an expanded population of MHC unrestricted lymphocytes that is greater in number than a reference population. In some embodiments, the expanded population of MHC unrestricted lymphocytes is greater in number than the isolated population of MHC unrestricted lymphocytes prior to the expansion step (e.g. at least 2-fold in number, at least 5-fold in number, at least 10-fold in number, at least 25-fold in number, at least 50-fold in number, at least 60-fold in number, at least 70-fold in number, at least 80-fold in number, at least 90-fold in number, at least 100-fold in number, at least 200-fold in number, at least 300-fold in number, at least 400-fold in number, at least 500-fold in number, at 600-fold in number, at least 1,000-fold in number, or more relative to the isolated population of MHC unrestricted lymphocytes prior to the expansion step). In one embodiment, the expanded population of MHC unrestricted lymphocytes is between 5-fold and 10-fold greater in number relative to the isolated population of MHC unrestricted lymphocytes prior to the expansion step. In a further embodiment, the expanded population is between 5-fold and 10-fold greater in number relative to the isolated population prior to expansion after a culture period as defined herein. In a yet further embodiment, the expanded population is between 5-fold and 10-fold greater in number than the isolated population prior to expansion after a culture period of 12 days. In another embodiment, the expanded population is between 5-fold and 10-fold greater in number relative to the isolated population prior to expansion after a culture period of 14 days. In one embodiment, the expanded population of MHC unrestricted lymphocytes is greater in number than a population cultured for the same length of time without the presence of the antibody or fragment thereof. In one embodiment, the expanded population of MHC unrestricted lymphocytes, in particular NK cells, is greater in number than a population cultured for the same length of time in the presence of IL-4.

Methods of the invention provide an expanded population of MHC unrestricted lymphocytes that may be characterised as described herein. For example, in some embodiments, at least 70% of the MHC unrestricted lymphocytes present in the cell population isolated using the method express CD56.

In some embodiments, the cell population isolated using the method comprises γδ T cells and at least 40% of the γδ T cells express CD56. In some embodiments at least about 45% of the γδ T cells express CD56. In some embodiments, at least 45%, such as at least 46%, 47%, 48%, 49%, 50%, 51%, 52%, 53%, 54%, 55%, 56%, 57%, 58%, 59%, 60% of the γδ T cells express CD56. In some embodiments, at least about 50% of the γδT cells express CD56. In some embodiments, at least about 55% of the γδT cells express CD56. In some embodiments, at least about 60% of the γδ T cells express CD56.

Methods of the invention may provide a population of MHC unrestricted lymphocytes that has a higher proportion of CD56^{bright} cells compared to a reference population (e.g. the starting material/sample). In some embodiments, there is at least a 40% increase in the number of CD56^{bright} γδ T cells present in the expanded composition. In some embodiments, there is at least a 45%, such as at least a 46%, 47%, 48%, 49%, 50%, 51%, 52%, 53%, 54%, 55%, 56%, 57%, 58%, 59%, 60% increase in the number of CD56^{bright} γδ T cells present in the expanded composition. In some embodiments, there is at least about a 50% increase in the number of CD56^{bright} γδ T cells present in the expanded composition. In some embodiments, there is at least about a 55% increase in the number of CD56^{bright} T cells present in the expanded composition. In some embodiments, there is at least about a 60% increase in the number of CD56^{bright} γδ T cells present in the expanded composition.

In some embodiments, at least about 40% of the γδ T cells present in the expanded composition are CD56^{bright}. In some embodiments, at least 45%, such as at least 46%, 47%, 48%, 49%, 50%, 51%, 52%, 53%, 54%, 55%, 56%, 57%, 58%, 59%, 60% of the γδ T cells present in the expanded composition are CD56^{bright}. In some embodiments, at least about 50% of the γδ T cells present in the expanded composition are CD56^{b}9. In some embodiments, at least about 55% of the γδ T cells present in the expanded composition are CD56^{bright}. In some embodiments, at least about 60% of the γδ T cells present in the expanded composition are CD56^{bright}.

In some embodiments, there is at least a 50% increase in the number of CD56^{bright} NK cells present in the expanded composition. In some embodiments, there is at least a 60%, such as at least a least 61%, 62%, 63%, 64%, 65%, 66%, 67%, 68%, 69%, 70%, 71%, 72%, 73%, 74%, 75%, 76%, 77%, 78%, 79%, 80% increase in the number of CD56^{bright} NK cells present in the expanded composition. In some embodiments, there is at least about a 70% increase in the number of CD56^{bright} NK cells present in the expanded composition. In some embodiments, there is at least about a 75% increase in the number of CD56^{bright} NK cells present in the expanded composition. In some embodiments, there is at least about a 80% increase in the number of CD56^{bright} NK cells present in the expanded composition.

In some embodiments at least about 50% of the NK cells present in the expanded composition are CD56^{bright}. In some embodiments, at least 60%, such as at least 61%, 62%, 63%, 64%, 65%, 66%, 67%, 68%, 69%, 70%, 71%, 72%, 73%, 74%, 75%, 76%, 77%, 78%, 79%, 80% of the NK cells present in the expanded composition are CD56^{bright}. In some embodiments, at least about 70% of the NK cells present in the expanded composition are CD56^{bright}. In some embodiments, at least about 75% of the NK cells present in the expanded composition are CD56^{bright}. In some embodiments, at least about 80% of the NK cells present in the expanded composition are CD56^{bright}.

Methods of expansion may provide an expanded population of MHC unrestricted lymphocytes that has a higher percentage of NK cells than a reference population. In some embodiments, the expanded population of MHC unrestricted lymphocytes contains at least about 30% NK cells, such as at least about 31%, 32%, 33%, 34%, 35%, 36%, 37%, 38%, 39%, 40%, 41%, 42%, 43%, 44%, 45%, 46%, 47%, 48%, 49% or 50% NK cells. In a further embodiment, the expanded population of MHC unrestricted lymphocytes contains at least about 40% NK cells. In some embodiments, the expanded population of MHC unrestricted lymphocytes contains less than about 80% NK cells, such as less than about 79%, 78%, 77%, 76%, 75%, 74%, 73%, 72%, 71%, 70%, 60%, 68%, 57%, 66%, 65%, 64%, 63%, 62%, 61%, 60%, 59%, 58%, 57%, 56%, 55%, 54%, 53%, 52%, 51% or 50% NK cells. In a further embodiment, the expanded population of MHC unrestricted lymphocytes contains less than about 60% NK cells. In some embodiments, the expanded population of MHC unrestricted lymphocytes contains between about 20% to 70% NK cells, such as about 30% to 60% NK cells. In a further embodiment, the expanded population of MHC unrestricted lymphocytes contains about 50% NK cells.

Methods of expansion may provide an expanded population of MHC unrestricted lymphocytes that has a higher percentage of γδ T cells than a reference population. In some embodiments, the expanded population of MHC unrestricted lymphocytes contains at least about 30% γδ T cells, such as at least about 31%, 32%, 33%, 34%, 35%, 36%, 37%, 38%, 39%, 40%, 41%, 42%, 43%, 44%, 45%, 46%, 47%, 48%, 49% or 50% γδ T cells. In a further embodiment, the expanded population of MHC unrestricted lymphocytes contains at least about 40% γδ T cells, such as greater than about 45% γδ T cells. In some embodiments, the expanded population of MHC unrestricted lymphocytes contains less than about 80% γδ T cells, such as less than about 79%, 78%, 77%, 76%, 75%, 74%, 73%, 72%, 71%, 70%, 60%, 68%, 57%, 66%, 65%, 64%, 63%, 62%, 61%, 60%, 59%, 58%, 57%, 56%, 55%, 54%, 53%, 52%, 51% or 50% γδ T cells. In a further embodiment, the expanded population of MHC unrestricted lymphocytes contains less than about 60% γδ T cells. In some embodiments, the expanded population of MHC unrestricted lymphocytes contains between about 30% to 60% γδ T cells. In a further embodiment, the expanded population of MHC unrestricted lymphocytes contains about 50% γδ T cells.

In some embodiments, the expanded population of MHC unrestricted lymphocytes contains at least about 10% Vδ1 T cells, such as at least about 11%, 12%, 13%, 14%, 15%, 16%, 17%, 18%, 19%, 20%, 21%, 22%, 23%, 24%, 25%, 26%, 27%, 28%, 29% or 30% Vδ1 T cells. In a further embodiment, the expanded population of Vδ1 T cells contains at least about 20% Vδ1 T cells. In some embodiments, the expanded population of MHC unrestricted lymphocytes contains between about 10% to 55% Vδ1 T cells, such as between about 10% to 30% Vδ1 T cells.

In some embodiments, the expanded population of MHC unrestricted lymphocytes contains at least about 5% Vδ2 T cells, such as at least about 6%, 7%, 8%, 9%, 10%, 11%, 12%, 13%, 14%, 15%, 16%, 17%, 18%, 19% or 20% Vδ2 T cells. In a further embodiment, the expanded population of Vδ1 T cells contains at least about 7% Vδ2 T cells. In some embodiments, the expanded population of MHC unrestricted lymphocytes contains between about 7% to 30% Vδ2 T cells.

In some embodiments, the expanded population of MHC unrestricted lymphocytes contains less than about 10% as T cells, such as less than about 5%, 4%, 3%, 2%, 1.5%, 1%, 0.5%, 0.2%, 0.1% or 0.05% as T cells. In a further embodiment, the expanded population of MHC unrestricted lymphocytes contains less than about 1% as T cells. T cells with as receptors are highly reactive, therefore suitable cell populations for administration to patients in the context of the present invention can only contain low levels of as T cells. The antibodies described herein may be used to selectively expand the MHC unrestricted lymphocytes which reduces the need for extensive purification methods after expansion in order to remove αβ T cells.

In some embodiments, at least 90% of the cell population isolated using the method consists of NK cells and γδ T cells and wherein at least 10% of the cells are Vδ1 T cells, at least 5% of the cells are Vδ2 T cells and at least 30% of the cells are NK cells.

An increase or decrease in expression of cell surface markers can be additionally or alternatively used to characterize one or more expanded populations of MHC unrestricted lymphocytes, including CD27 and/or NKp30. In some embodiments, the expanded population of Vδ1 T cells present in the MHC unrestricted lymphocyte population expresses a lower level of CD27 compared to a reference population (such as a population not expanded using a method of the invention). In some embodiments, the expanded population of Vδ1 T cells present in the MHC unrestricted lymphocyte population expresses a low level of CD27 (CD27^{low}). For example, less than about 70%, such as less than about 50%, 40% or 30% of the expanded population of Vδ1 T cells expresses CD27 (i.e. CD27+). In some embodiments, the expanded population of Vδ1 T cells present in the MHC unrestricted lymphocyte population expresses a higher level of NKp30 compared to a reference population (such as a population not expanded using a method of the invention). For example, more than about 10%, such as more than about 15%, 20% or 25% of the expanded population of Vδ1 T cells expresses NKp30 (i.e. NKp30+).

In some embodiments, the expanded population of Vδ1 T cells present in the MHC unrestricted lymphocyte population expresses a high level of NKG2D (NKG2D^{high}). For example, more than about 80%, such as more than about 85%, 90% or 95% of the expanded population of Vδ1 T cells expresses NKG2D (i.e. NKG2D+).

Numerous basal culture media suitable for use in the proliferation of γδ T cells are available, in particular medium, such as CTS OpTmizer (Thermo Fisher) AIM-V, Iscoves medium and RPMI-1840 (Life Technologies), EXVIVO-10, EXVIVO-15 or EXVIVO-20 (Lonza), optionally in the presence of serum or plasma. The medium may be supplemented with other media factors as defined herein, such as serum, serum proteins and selective agents, such as antibiotics. For example, in some embodiments, RPMI-1640 medium containing 2 mM glutamine, 10% FBS, 10 mM HEPES, pH 7.2, 1% penicillin-streptomycin, sodium pyruvate (1 mM; Life Technologies), non-essential amino acids (e.g. 100 μM Gly, Ala, Asn, Asp, Glu, Pro and Ser; 1×MEM non-essential amino acids (Life Technologies)), and 10 μl/L β-mercaptoethanol. In an alternative embodiment, AIM-V medium may be supplemented with CTS Immune serum replacement and amphotericin B. Conveniently, cells are cultured at 37° C. in a humidified atmosphere containing 5% CO₂ in a suitable culture medium during culturing.

The medium may be further supplemented with factors which specifically support the expansion of particular cells, such as NK cells and/or Vδ2 T cells. Thus, in one embodiment the medium comprises an NK cell and/or Vδ2 T cell supplement. Examples of medium supplements include the NK supplement from Miltenyi Biotech. The presence of such supplement(s) may enhance the proliferation potential of the target cells, thus increasing the proportion of such cells in the expanded population.

As described herein, antibodies (or fragments thereof) may be applied to cell population which has been obtained from a sample. In one embodiment, the cell population is isolated from a sample prior to administering the anti-Vδ1 antibody or fragment thereof.

References herein to “isolation” or “isolating” of cells, in particular of γδ T cells, refer to methods or processes wherein cells are removed, separated, purified, enriched or otherwise taken out from a tissue or a pool of cells. It will be appreciated that such references include the terms “separated”, “removed”, “purified”, “enriched” and the like. Isolation of γδ T cells includes the isolation or separation of cells from an intact non-haematopoietic tissue sample or from the stromal cells of the non-haematopoietic tissue (e.g. fibroblasts or epithelial cells). Such isolation may alternatively or additionally comprise the isolation or separation of γδ T cells from other haematopoietic cells (e.g. as T cells or other lymphocytes). Isolation may be for a defined period of time, for example starting from the time the tissue explant or biopsy is placed in the isolation culture and ending when the cells are collected from culture, such as by centrifugation or other means for transferring the isolated cell population to expansion culture or used for other purposes, or the original tissue explant or biopsy is removed from the culture. The isolation step may be for at least about 3 days to about 45 days. In one embodiment, the isolation step is for at least about 10 days to at least 28 days. In a further embodiment, the isolation step is for at least 14 days to at least 21 days. The isolation step may therefore be for at least 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 28 days, 27 days, 28 days, 29 days, 30 days, 31 days, 32 days, about 35 days, about 40 days, or about 45 days. It can be appreciated that although cell proliferation may not be substantial during this isolation step, it is not necessarily absent Indeed for someone skilled in the art it is recognized that isolated cells may also start to divide to generate a plurality of such cells within the isolation vessel containing the sample.

The cell population may be obtained by any suitable method that allows isolation of lymphocytes, in particular Vδ1 T cells, from human or non-human animal samples, such as a non-haematopoietic tissue sample. One such method is set out in Clark et al. (2006) J. Invest. Dermatol. 126(5): 1059-70, which describes a three-dimensional skin explant protocol for isolating lymphocytes from human skin. An explant may be adhered to a synthetic scaffold to facilitate lymphocyte egress from the explant onto the scaffold. A synthetic scaffold refers to a non-native three-dimensional structure suitable to support cell growth. Synthetic scaffolds may be constructed from materials such as polymers (e.g. natural or synthetic polymers, e.g. poly vinyl pyrolidones, polymethylmethacrylate, methyl cellulose, polystyrene, polypropylene, polyurethane), ceramics (e.g. tricalcium phosphate, calcium aluminate, calcium hydroxyapatite), or metals (tantalum, titanium, platinum and metals in the same element group as platinum, niobium, hafnium, tungsten, and combinations of alloys thereof). Biological factors (e.g. collagens (e.g. collagen I or collagen II), fibronectins, laminins, integrins, angiogenic factors, anti-inflammatory factors, glycosaminoglycans, vitrogens, antibodies and fragments thereof, cytokines may be coated onto the scaffold surface or encapsulated within the scaffold material to enhance cell adhesion, migration, survival, or proliferation, according to methods known in the art. This and other methods can be used to isolate a cell population from a number of other non-haematopoietic tissue types, e.g. gut, prostate and breast. Other examples of suitable methods of isolation utilise “crawl-out” methods which may include the culturing of the cell population and/or sample in the presence of cytokines and/or chemokines sufficient to induce the isolation or separation of immune cells, in particular MHC unrestricted lymphocytes.

Non-haematopoietic tissue resident lymphocytes can be harvested and separated from stromal cells, such as dermal fibroblasts, e.g. by firm pipetting. The lymphocyte harvest may further be washed through a 40 μm nylon mesh in order to retain fibroblast aggregates that may have become loose during the process. Lymphocytes may also be isolated using fluorescent or magnetic associated cell sorting using, for example, CD45 antibodies.

References herein to “culturing” include the addition of the sample, including isolated, separated, removed, purified or enriched cells from the sample, to media comprising growth factors and/or essential nutrients required and/or preferred by the cells and/or sample. It will be appreciated that such culture conditions may be adapted according to the cells or cell population to be isolated from the sample or may be adapted according to the cells or cell population to be isolated and expanded from the sample.

Culture media may additionally include other ingredients that can assist in the growth and expansion of the γδ T cells. Examples of other ingredients that may be added, include, but are not limited to, plasma or serum, purified proteins such as albumin, a lipid source such as low density lipoprotein (LDL), vitamins, amino acids, steroids and any other supplements supporting or promoting cell growth and/or survival.

### Antibodies or Fragments Thereof

Antibodies of use in the methods provided herein are antibodies or fragments thereof capable of specifically binding to the delta variable 1 chain (Vδ1) of a γδ T Cell Receptor (TCR). In one embodiment, the anti-Vδ1 antibody or fragment thereof is an activating anti-Vδ1 antibody or fragment thereof. It has surprisingly been found that culturing a cell population in the presence of an anti-Vδ1 antibody under certain culture conditions was able to simultaneously enrich for all MHC unrestricted lymphocytes, and not just Vδ1 T cells as expected. In some embodiments, the anti-Vδ1 antibody is used at a concentration of 42 ng/ml. Thus, in one embodiment culturing step of the method described herein is performed in the presence of 42 ng/ml anti-Vδ1 antibody.

In one embodiment, the antibody or fragment thereof is an scFv, Fab, Fab′, F(ab′)2, Fv, variable domain (e.g. VH or VL), diabody, minibody or monoclonal antibody. In a further embodiment, the antibody or fragment thereof is an scFv.

Antibodies described herein can be of any class, e.g. IgG, IgA, IgM, IgE, IgD, or isotypes thereof, and can comprise a kappa or lambda light chain. In one embodiment, the antibody is an IgG antibody, for example, at least one of isotypes, IgG1, IgG2, IgG3 or IgG4.

In a further embodiment, the antibody may be in a format, such as an IgG format, that has been modified to confer desired properties, such as having the Fc mutated to reduce effector function, extend half life, alter ADCC, or improve hinge stability. Such modifications are well known in the art.

In one embodiment, the antibody or fragment thereof is human. Thus, the antibody or fragment thereof may be derived from a human immunoglobulin (Ig) sequence. The CDR, framework and/or constant region of the antibody (or fragment thereof) may be derived from a human Ig sequence, in particular a human IgG sequence. The CDR, framework and/or constant region may be substantially identical for a human Ig sequence, in particular a human IgG sequence. An advantage of using human antibodies is that they are low or non-immunogenic in humans.

An antibody or fragment thereof can also be chimeric, for example a mouse-human antibody chimera.

Alternatively, the antibody or fragment thereof is derived from a non-human species, such as a mouse. Such non-human antibodies can be modified to increase their similarity to antibody variants produced naturally in humans, thus the antibody or fragment thereof can be partially or fully humanised. Therefore, in one embodiment, the antibody or fragment thereof is humanised.

### Antibodies Targeted to Epitopes

Provided herein are antibodies (or fragments thereof) which bind to an epitope of the Vδ1 chain of a γδ TCR. Such binding may optionally have an effect on γδ TCR activity, such as activation or inhibition.

In one embodiment, the epitope may be an activating epitope of a γδ T cell. An “activating” epitope can include, for example, stimulating a TCR function, such as degranulation, TCR downregulation, cytotoxicity, proliferation, mobilisation, increased survival or resistance to exhaustion, intracellular signaling, cytokine or growth factor secretion, phenotypic change, or a change in gene expression. For example, the binding of the activating epitope may stimulate expansion (i.e. proliferation) of the γδ T cell population, preferably the Vδ1 T cell population. Accordingly, these antibodies can be used to modulate γδ T cell activation, and, thereby, to modulate the immune response. Therefore, in one embodiment, binding of the activating epitope downregulates the γδ TCR. In an additional or alternative embodiment, binding of the activating epitope activates degranulation of the γδ T cell. In a further additional or alternative embodiment, binding of the activating epitope activates γδ T cell killing.

Alternatively, the antibodies (or fragments thereof) may have a blocking effect by prevention of the binding or interaction of another antibody or molecule. In one embodiment, the present invention provides isolated antibodies or fragments thereof that block Vδ1 and prevent TCR binding (e.g. through steric hinderance). By blocking Vδ1, the antibody may prevent TCR activation and/or signalling. The epitope may be an inhibitory epitope of a γδ T cell. An “inhibitory” epitope can include, for example, blocking TCR function, thereby inhibiting TCR activation.

The epitope is preferably comprised of at least one extracellular, soluble, hydrophillic, external or cytoplasmic portion of the Vδ1 chain of a γδ TCR.

In particular, the epitope does not comprise an epitope found in a hypervariable region of the Vδ1 chain of the γδ TCR, in particular CDR3 of the Vδ1 chain. In a preferred embodiment, the epitope is within the non-variable region of the Vδ1 chain of the γδ TCR. It will be appreciated that such binding allows for the unique recognition of the Vδ1 chain without the restriction to the sequences of the TCR which are highly variable (in particular CDR3). Various γδ TCR complexes which recognise MHC-like peptides or antigen may be recognised in this way, solely by presence of the Vδ1 chain. As such, it will be appreciated that any Vδ1 chain-comprising γδ TCR may be recognised using the antibodies or fragments thereof as defined herein, irrespective of the specificity of the γδ TCR. In one embodiment, the epitope comprises one or more amino acid residues within amino acid regions 1-24 and/or 35-90 of SEQ ID NO: 1, e.g. the portions of the Vδ1 chain which are not part of the CDR1 and/or CDR3 sequences. In one embodiment, the epitope does not comprise amino acid residues within amino acid region 91-105 (CDR3) of SEQ ID NO: 1.

In a similar manner to the well characterised as T cells, γδ T cells utilize a distinct set of somatically rearranged variable (V), diversity (D), joining (J), and constant (C) genes, although γδ T cells contain fewer V, D, and J segments than αβ T cells. In one embodiment, the epitope bound by the antibodies (or fragments thereof) does not comprise an epitope found in the J region of the Vδ1 chain (e.g. one of the four J regions encoded in the human delta one chain germline: SEQ ID NO: 131 (J1*0) or 132 (J2*0) or 133 (J3*0) or 134 (J4*0)). In one embodiment, the epitope bound by the antibodies (or fragments thereof) does not comprise an epitope found in the C-region of the Vδ1 chain (e.g. SEQ ID NO: 135 (C1*0) which contains the C-terminal juxtamembrane/transmembrane regions). In one embodiment, the epitope bound by the antibodies (or fragments thereof) does not comprise an epitope found in the N-terminal leader sequence of the Vδ1 chain (e.g. SEQ ID NO:129). The antibody or fragment may therefore only bind in the V region of the Vδ1 chain (e.g. SEQ ID NO: 130). Thus, in one embodiment, the epitope consists of an epitope in the V region of the γδ TCR (e.g. amino acid residues 1-90 of SEQ ID NO: 1).

Reference to the epitope are made in relation to the Vδ1 sequence derived from the sequence described in Luoma et al. (2013) Immunity 39: 1032-1042, and RCSB Protein Data Bank entries: 4MNH and 3OMZ, shown as SEQ ID NO: 1:

(SEQ ID NO: 1)
AQKVTQAQSSVSMPVRKAVTLNCLYETSWWSYYIFWYKQLPSKE
MIFLIRQGSDEQNAKSGRYSVNFKKAAKSVALTISALQLEDSAK
YFCALGESLTRADKLIFGKGTRVTVEPNIQNPDPAVYQLRDSKS
SDKSVCLFTDFDSQTNVSQSKDSDVYITDKTVLDMRSMDFKSNS
AVAWSNKSDFACANAFNNSIIPEDTFFPSPESS

SEQ ID NO: 1 represents a soluble TCR comprising a V region (also referred to as the variable domain), a D region, a J region and a TCR constant region. The V region comprises amino acid residues 1-90, the D region comprises amino acid residues 91-104, the J region comprises amino acid residues 105-115 and the constant region comprises amino acid residues 116-209. Within the V region, CDR1 is defined as amino acid residues 25-34 of SEQ ID NO: 1, CDR2 is defined as amino acid residues 50-54 of SEQ ID NO: 1, and CDR3 is defined as amino acid residues 93-104 of SEQ ID NO: 1 (Xu et al., PNAS USA 108(6):2414-2419 (2011)).

Therefore, in one embodiment, the isolated antibody or fragment thereof binds to an epitope of a variable delta 1 (Vδ1) chain of a γδ T cell receptor (TCR) comprising one or more amino acid residues within amino acid regions:
(i) 3-20 of SEQ ID NO: 1; and/or
(ii) 37-77 of SEQ ID NO: 1.

Epitope mapping of the antibodies described herein was conducted as described in Examples 1 and 9 of PCT Application No. PCT/GB20201051956, which are herein incorporated by reference.

In a further embodiment, antibodies or fragments thereof additionally recognize the polymorphic V region comprising amino acid residues 1-90 epitope of SEQ ID NO:128. Hence, amino acids 1-90 of SEQ ID NO:1 and the polymorphic germline variant sequence (amino acids 1-90 SEQ ID NO:128) may be considered interchangeable when defining epitopes described herein. Antibodies of the invention can recognize both variants of this germline sequence. By way of example, where it is stated that antibodies or fragments thereof as defined herein recognize epitopes comprising one or more amino acid residues within amino acid regions 1-24 and/or 35-90 of SEQ ID NO:1 this also refers to the same regions of SEQ ID NO:128; specifically amino acid regions 1-24 and/or 35-90 of SEQ ID NO:128.

In one embodiment, antibodies or fragments thereof recognize one or more amino acid residues within amino acid regions 1-90 of SEQ ID NO:1 and the equivalently located amino acids of regions 1-90 in SEQ ID NO:128. More specifically, in one embodiment antibodies or fragments thereof as defined herein recognize a human germline epitope wherein said germline encodes either an alanine (A) or valine (V) at position 71 of SEQ ID NO:1.

In one embodiment, the epitope comprises one or more, such as two, three, four, five, six, seven, eight, nine, ten or more amino acid residues within the described regions.

In a further embodiment, the epitope comprises one or more (such as 5 or more, such as 10 or more) amino acid residues within amino acid region 3-20 of SEQ ID NO: 1. In an alternative embodiment, the epitope comprises one or more (such as 5 or more, such as 10 or more) amino acid residues within amino acid region 37-77 of SEQ ID NO: 1 (such as amino acid region 50-54). In a yet further embodiment, the epitope comprises one or more (such as 5 or more, such as 10 or more) amino acid residues within amino acid region 3-20 (such as 5-20 or 3-17) and one or more (such as 5 or more, such as 10 or more) amino acid residues within amino acid region 37-77 (such as 62-77 or 62-69) of SEQ ID NO: 1.

It will be further understood that said antibody (or fragment thereof) does not need to bind to all amino acids within the defined range. Such epitopes may be referred to as linear epitopes. For example, an antibody which binds to an epitope comprising amino acid residues within amino acid region 5-20 of SEQ ID NO: 1, may only bind with one or more of the amino acid residues in said range, e.g. the amino acid residues at each end of the range (i.e. amino acids 5 and 20), optionally including amino acids within the range (i.e. amino acids 5, 9, 16 and 20).

In one embodiment, the epitope comprises at least one of amino acid residues 3, 5, 9, 10, 12, 16, 17, 20, 37, 42, 50, 53, 59, 62, 64, 68, 69, 72 or 77 of SEQ ID NO: 1. In further embodiments, the epitope comprises one, two, three, four, five, six, seven, eight, nine, ten, eleven or twelve amino acids selected from amino acid residues 3, 5, 9, 10, 12, 16, 17, 20, 37, 42, 50, 53, 59, 62, 64, 68, 69, 72 or 77 of SEQ ID NO: 1.

In one embodiment, the epitope comprises one or more amino acid residues within the following amino acid regions of SEQ ID NO: 1 (or SEQ ID NO:128, as described above):
(i) 3-17;
(ii) 5-20;
(iii) 37-53;
(iv) 50-64;
(v) 59-72;
(vi) 59-77;
(vii) 62-69; and/or
(viii) 62-77.

In a further embodiment, the epitope comprises one or more amino acid residues within amino acid regions: 5-20 and 62-77; 50-64; 37-53 and 59-72; 59-77; or 3-17 and 62-69, of SEQ ID NO: 1. In a further embodiment, the epitope consists of one or more amino acid residues within amino acid regions: 5-20 and 62-77; 50-64; 37-53 and 59-72; 59-77; or 3-17 and 62-69, of SEQ ID NO: 1.

In a further embodiment, the epitope comprises amino acid residues: 3, 5, 9, 10, 12, 16, 17, 62, 64, 68 and 69 of SEQ ID NO: 1, or suitably consists of amino acid residues: 3, 5, 9, 10, 12, 16, 17, 62, 64, 68 and 69 of SEQ ID NO: 1. In a further embodiment, the epitope comprises amino acid residues: 5, 9, 16, 20, 62, 64, 72 and 77 of SEQ ID NO: 1, or suitably consists of amino acid residues: 5, 9, 16, 20, 62, 64, 72 and 77 of SEQ ID NO: 1. In yet further embodiment, the epitope comprises the amino acid residues: 37, 42, 50, 53, 59, 64, 68, 69, 72, 73 and 77 of SEQ ID NO: 1, or suitably consists of amino acid residues: 37, 42, 50, 53, 59, 64, 68, 69, 72, 73 and 77 of SEQ ID NO: 1. In a further embodiment, the epitope comprises the amino acid residues: 50, 53, 59, 62 and 64 of SEQ ID NO: 1, or suitably consists of amino acid residues: 50, 53, 59, 62 and 64 of SEQ ID NO: 1. In a further embodiment, the epitope comprises amino acid residues: 59, 60, 68 and 72 of SEQ ID NO: 1, or suitably consists of amino acid residues: 59, 60, 68 and 72 of SEQ ID NO: 1.

In one embodiment, the epitope comprises one or more amino acid residues within amino acid regions 5-20 and/or 62-77 of SEQ ID NO: 1. In a further embodiment, the epitope consists of one or more amino acid residues within amino acid regions 5-20 and 62-77 of SEQ ID NO: 1. In an alternative further embodiment, the epitope comprises one or more amino acid residues within amino acid regions 5-20 or 62-77 of SEQ ID NO: 1. Antibodies or fragments thereof having such epitopes may have some or all of the sequences of 1245_P01_E07, or such antibodies or fragments thereof may be derived from 1245_P01_E07. For example, antibodies or fragments thereof having one or more CDR sequences of 1245_P01_E07 or one or both of the VH and VL sequences of 1245_P01_E07 may bind such epitopes.

In one embodiment, the epitope comprises one or more amino acid residues within amino acid region 50-84 of SEQ ID NO: 1. In a further embodiment, the epitope consists of one or more amino acid residues within amino acid region 50-84 of SEQ ID NO: 1. Antibodies or fragments thereof having such epitopes may have some or all of the sequences of 1252_P01_C08, or such antibodies or fragments thereof may be derived from 1252_P01_C08. For example, antibodies or fragments thereof having one or more CDR sequences of 1252_P01_C08 or one or both of the VH and VL sequences of 1252_P01_C08 may bind such epitopes.

In one embodiment, the epitope comprises one or more amino acid residues within amino acid regions 37-53 and/or 59-77 of SEQ ID NO: 1. In a further embodiment, the epitope consists of one or more amino acid residues within amino acid regions 37-53 and 59-77 of SEQ ID NO: 1. In an alternative further embodiment, the epitope comprises one or more amino acid residues within amino acid regions 37-53 or 59-77 of SEQ ID NO: 1. Antibodies or fragments thereof having such epitopes may have some or all of the sequences of 1245_P02_G04, or such antibodies or fragments thereof may be derived from 1245_P02_G04. For example, antibodies or fragments thereof having one or more CDR sequences of 1245_P02_G04 or one or both of the VH and VL sequences of 1245_P02_G04 may bind such epitopes.

In one embodiment, the epitope comprises one or more amino acid residues within amino acid region 59-72 of SEQ ID NO: 1. In a further embodiment, the epitope consists of one or more amino acid residues within amino acid region 59-72 of SEQ ID NO: 1. Antibodies or fragments thereof having such epitopes may have some or all of the sequences of 1251_P02_C05, or such antibodies or fragments thereof may be derived from 1251_P02_C05. For example, antibodies or fragments thereof having one or more CDR sequences of 1251_P02_C05 or one or both of the VH and VL sequences of 1251_P02_C05 may bind such epitopes.

In one embodiment, the epitope does not comprise amino acid residues within amino acid region 11-21 of SEQ ID NO: 1. In one embodiment, the epitope does not comprise amino acid residues within amino acid region 21-28 of SEQ ID NO: 1. In one embodiment, the epitope does not comprise amino acid residues within the amino acid region 59 and 60 of SEQ ID NO: 1. In one embodiment, the epitope does not comprise amino acid residues within the amino acid region 87-82 of SEQ ID NO: 1.

In one embodiment, the epitope is not the same epitope bound by a commercially available anti-Vδ1 antibody, such as TS-1 or TS8.2. As described in WO2017197347, binding of TS-1 and TS8.2 to soluble TCRs was detected when the 61 chain included Vδ1 J1 and Vδ1 J2 sequences but not to the Vδ1 J3 chain, indicating that the binding of TS-1 and TS8.2 involved critical residues in the delta J1 and delta J2 region.

References to “within” herein include the extremities of the define range. For example, “within amino acid regions 5-20” refers to all of amino acid resides from and including residue 5 up to and including residue 20.

Various techniques are known in the art to establish which epitope is bound by an antibody. Exemplary techniques include, for example, routine cross-blocking assays, alanine scanning mutational analysis, peptide blot analysis, peptide cleavage analysis crystallographic studies and NMR analysis. In addition, methods such as epitope excision, epitope extraction and chemical modification of antigens can be employed. Another method that can be used to identify the amino acids within a polypeptide with which an antibody interacts is hydrogen/deuterium exchange detected by mass spectrometry (as described in Example 9).

In general terms, the hydrogen/deuterium exchange method involves deuterium-labelling the protein of interest, followed by binding the antibody to the deuterium-labelled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labelled residues which correspond to the specific amino acids with which the antibody interacts.

### Antibody Sequences

The isolated anti-Vδ1 antibodies, or fragments thereof, may be described with reference to their CDR sequences.

In one embodiment, the anti-Vδ1 antibody or fragment thereof comprises one or more of:
a CDR3 comprising a sequence having at least 80% sequence identity with any one of SEQ ID NOs: 2-25;
a CDR2 comprising a sequence having at least 80% sequence identity with any one of SEQ ID NOs: 26-37 and SEQUENCES: A1-A12; and/or
a CDR1 comprising a sequence having at least 80% sequence identity with any one of SEQ ID NOs: 38-81.

In one embodiment, the isolated anti-Vδ1 antibody or fragment thereof comprises a CDR3 comprising a sequence having at least 80% sequence identity with any one of SEQ ID NOs: 2-25. In one embodiment, the antibody or fragment thereof comprises a CDR2 comprising a sequence having at least 80% sequence identity with any one of SEQ ID NOs: 26-37 and SEQUENCES: A1-A12 (of Table 2). In one embodiment, the antibody or fragment thereof comprises a CDR1 comprising a sequence having at least 80% sequence identity with any one of SEQ ID NOs: 38-81.

In one embodiment, the antibody or fragment thereof comprises a CDR3 comprising a sequence having at least 85%, 90%, 95%, 97%, 98% or 99% sequence identity with any one of SEQ ID NOs: 2-25. In one embodiment, the antibody or fragment thereof comprises a CDR2 comprising a sequence having at least 85%, 90%, 95%, 97%, 98% or 99% sequence identity with any one of SEQ ID NOs: 26-37 and SEQUENCES: A1-A12 (of Table 2). In one embodiment, the antibody or fragment thereof comprises a CDR1 comprising a sequence having at least 85%, 90%, 95%, 97%, 98% or 99% sequence identity with any one of SEQ ID NOs: 38-61.

In one embodiment, the antibody or fragment thereof comprises a CDR3 consisting of a sequence having at least 85%, 90%, 95%, 97%, 98% or 99% sequence identity with any one of SEQ ID NOs: 2-25. In one embodiment, the antibody or fragment thereof comprises a CDR2 consisting of a sequence having at least 85%, 90%, 95%, 97%, 98% or 99% sequence identity with any one of SEQ ID NOs: 26-37 and SEQUENCES: A1-A12 (of Table 2). In one embodiment, the antibody or fragment thereof comprises a CDR1 consisting of a sequence having at least 85%, 90%, 95%, 97%, 98% or 99% sequence identity with any one of SEQ ID NOs:38-61.

In one embodiment the antibody or fragment thereof comprises a VH region comprising a CDR3 sequence sharing at least 80% sequence identity with any one of SEQ ID NOs: 2-13 and/or a VL region comprising a CDR3 comprising a sequence having at least 80% sequence identity with any one of SEQ ID NOs: 14-25. In one embodiment, the antibody or fragment thereof comprises a VH region comprising a CDR3 consisting of a sequence having at least 80% sequence identity with any one of SEQ ID NOs: 2-13 and/or a VL region comprising a CDR3 consisting of a sequence having at least 80% sequence identity with any one of SEQ ID NOs: 14-25.

In one embodiment, the antibody or fragment thereof comprises a VH region comprising a CDR3 comprising a sequence having at least 90% sequence identity with any one of SEQ ID NOs: 2-13 and/or a VL region comprising a CDR3 comprising a sequence having at least 90% sequence identity with any one of SEQ ID NOs: 14-25. In one embodiment, the antibody or fragment thereof comprises a VH region comprising a CDR3 consisting of a sequence having at least 90% sequence identity with any one of SEQ ID NOs: 2-13 and/or a VL region comprising a CDR3 consisting of a sequence having at least 90% sequence identity with any one of SEQ ID NOs: 14-25.

In one embodiment, the antibody or fragment thereof comprises a VH region comprising a CDR3 comprising a sequence having at least 95% sequence identity with any one of SEQ ID NOs: 2-13 and/or a VL region comprising a CDR3 comprising a sequence having at least 95% sequence identity with any one of SEQ ID NOs: 14-25. In one embodiment, the antibody or fragment thereof comprises a VH region comprising a CDR3 consisting of a sequence having at least 95% sequence identity with any one of SEQ ID NOs: 2-13 and/or a VL region comprising a CDR3 consisting of a sequence having at least 95% sequence identity with any one of SEQ ID NOs: 14-25.

In one embodiment, the antibody or fragment thereof comprises a VH region comprising a CDR3 comprising a sequence having at least 80% sequence identity with any one of SEQ ID NOs: 2-13 and a VL region comprising a CDR3 comprising a sequence having at least 80% sequence identity with any one of SEQ ID NOs: 14-25. In one embodiment, the antibody or fragment thereof comprises a VH region comprising a CDR3 consisting of a sequence having at least 80% sequence identity with any one of SEQ ID NOs: 2-13 and a VL region comprising a CDR3 consisting of a sequence having at least 80% sequence identity with any one of SEQ ID NOs: 14-25.

In one embodiment, the antibody or fragment thereof, which comprises a VH region comprising a CDR3 comprising a sequence having at least 80% sequence identity with any one of SEQ ID NOs: 2-7, in particular 2-8, such as 2, 3 or 4 and a VL region comprising a CDR3 comprising a sequence having at least 80% sequence identity with any one of SEQ ID NOs: 14-19, in particular 14-18, such as 14, 15 or 16. In one embodiment, the antibody or fragment thereof, which comprises a VH region comprising a CDR3 consisting of a sequence having at least 80% sequence identity with any one of SEQ ID NOs: 2-7, in particular 2-6, such as 2, 3 or 4 and a VL region comprising a CDR3 consisting of a sequence having at least 80% sequence identity with any one of SEQ ID NOs: 14-19, in particular 14-18, such as 14, 15 or 16.

In one embodiment, the antibody or fragment thereof, which comprises a VH region comprising a CDR3 comprising a sequence having at least 90% sequence identity with any one of SEQ ID NOs: 2-7, in particular 2-6, such as 2, 3 or 4 and/or a VL region comprising a CDR3 comprising a sequence having at least 90% sequence identity with any one of SEQ ID NOs: 14-19, in particular 14-18, such as 14, 15 or 16. In one embodiment, the antibody or fragment thereof, which comprises a VH region comprising a CDR3 consisting of a sequence having at least 90% sequence identity with any one of SEQ ID NOs: 2-7, in particular 2-6, such as 2, 3 or 4 and/or a VL region comprising a CDR3 consisting of a sequence having at least 90% sequence identity with any one of SEQ ID NOs: 14-19, in particular 14-18, such as 14, 15 or 16.

In one embodiment, the antibody or fragment thereof, which comprises a VH region comprising a CDR3 comprising a sequence having at least 95% sequence identity with any one of SEQ ID NOs: 2-7, in particular 2-8, such as 2, 3 or 4 and/or a VL region comprising a CDR3 comprising a sequence having at least 95% sequence identity with any one of SEQ ID NOs: 14-19, in particular 14-18, such as 14, 15 or 18. In one embodiment, the antibody or fragment thereof, which comprises a VH region comprising a CDR3 consisting of a sequence having at least 95% sequence identity with any one of SEQ ID NOs: 2-7, in particular 2-8, such as 2, 3 or 4 and/or a VL region comprising a CDR3 consisting of a sequence having at least 95% sequence identity with any one of SEQ ID NOs: 14-19, in particular 14-18, such as 14, 15 or 16.

In one embodiment, the antibody or fragment thereof, which comprises a VH region comprising a CDR3 comprising a sequence having at least 80% sequence identity with any one of SEQ ID NOs: 8-13, in particular 8, 9, 10 or 11 and/or a VL region comprising a CDR3 comprising a sequence having at least 80% sequence identity with any one of SEQ ID NOs: 20-25, in particular 20, 21, 22 or 23. In one embodiment, the antibody or fragment thereof, which comprises a VH region comprising a CDR3 consisting of a sequence having at least 80% sequence identity with any one of SEQ ID NOs: 8-13, in particular 8, 9, 10 or 11 and/or a VL region comprising a CDR3 consisting of a sequence having at least 80% sequence identity with any one of SEQ ID NOs: 20-25, in particular 20, 21, 22 or 23.

In one embodiment, the antibody or fragment thereof, which comprises a VH region comprising a CDR3 comprising a sequence having at least 90% sequence identity with any one of SEQ ID NOs: 8-13, in particular 8, 9, 10 or 11 and/or a VL region comprising a CDR3 comprising a sequence having at least 90% sequence identity with any one of SEQ ID NOs: 20-25, in particular 20, 21, 22 or 23. In one embodiment, the antibody or fragment thereof, which comprises a VH region comprising a CDR3 consisting of a sequence having at least 90% sequence identity with any one of SEQ ID NOs: 8-13, in particular 8, 9, 10 or 11 and/or a VL region comprising a CDR3 consisting of a sequence having at least 90% sequence identity with any one of SEQ ID NOs: 20-25, in particular 20, 21, 22 or 23.

In one embodiment, the antibody or fragment thereof, which comprises a VH region comprising a CDR3 comprising a sequence having at least 95% sequence identity with any one of SEQ ID NOs: 8-13, in particular 8, 9, 10 or 11 and/or a VL region comprising a CDR3 comprising a sequence having at least 95% sequence identity with any one of SEQ ID NOs: 20-25, in particular 20, 21, 22 or 23. In one embodiment, the antibody or fragment thereof, which comprises a VH region comprising a CDR3 consisting of a sequence having at least 95% sequence identity with any one of SEQ ID NOs: 8-13, in particular 8, 9, 10 or 11 and/or a VL region comprising a CDR3 consisting of a sequence having at least 95% sequence identity with any one of SEQ ID NOs: 20-25, in particular 20, 21, 22 or 23.

Embodiments which refer herein to “at least 80%” or “80% or greater”, will be understood to include all values equal to or greater than 80%, such as 85%, 90%, 95%, 97%, 98%, 99% or 100% sequence identity. In one embodiment, the antibody or fragment thereof comprises at least 85%, such as at least 90%, at least 95%, at least 97%, at least 98% or at least 99% sequence identity to the specified sequence.

Instead of percentage sequence identity, the embodiments may also be defined with one or more amino acid changes, for examples one or more additions, substitutions and/or deletions. In one embodiment, the sequence may comprise up to five amino acid changes, such as up to three amino acid changes, in particular up to two amino acid changes. In a further embodiment, the sequence may comprise up to five amino acid substitutions, such as up to three amino acid substitutions, in particular up to one or two amino acid substitutions. For example, CDR3 of the antibody or fragment thereof comprises or more suitably consists of a sequence having no more than 2, more suitably no more than 1 substitution(s) compared to any one of SEQ ID NOs: 2-25.

Suitably any residues of CDR1, CDR2 or CDR3 differing from their corresponding residues in SEQ ID NO: 2-61 and SEQUENCES: A1-A12 are conservative substitutions with respect to their corresponding residues. For example, any residues of CDR3 differing from their corresponding residues in SEQ ID NOs: 2-25 are conservative substitutions with respect to their corresponding residues.

In one embodiment, the antibody or fragment thereof comprises:
(i) a VH region comprising a CDR3 comprising a sequence having at least 80% sequence identity with any one of SEQ ID NOs: 2-13;
(ii) a VH region comprising a CDR2 comprising a sequence having at least 80% sequence identity with any one of SEQ ID NOs: 26-37;
(iii) a VH region comprising a CDR1 comprising a sequence having at least 80% sequence identity with any one of SEQ ID NOs: 38-49;
(iv) a VL region comprising a CDR3 comprising a sequence having at least 80% sequence identity with any one of SEQ ID NOs: 14-25;
(v) a VL region comprising a CDR2 comprising a sequence having at least 80% sequence identity with any one of SEQUENCES: A1-A12; and/or
(vi) a VL region comprising a CDR1 comprising a sequence having at least 80% sequence identity with any one of SEQ ID NOs: 50-81.

In one embodiment, the antibody or fragment thereof comprises a heavy chain with:
(i) a VH region comprising a CDR3 comprising a sequence having at least 80% sequence identity with any one of SEQ ID NOs: 2-13;
(ii) a VH region comprising a CDR2 comprising a sequence having at least 80% sequence identity with any one of SEQ ID NOs: 26-37; and
(iii) a VH region comprising a CDR1 comprising a sequence having at least 80% sequence identity with any one of SEQ ID NOs: 38-49.

In one embodiment, the antibody or fragment thereof comprises a light chain with:
(i) a VL region comprising a CDR3 comprising a sequence having at least 80% sequence identity with any one of SEQ ID NOs: 14-25;
(ii) a VL region comprising a CDR2 comprising a sequence having at least 80% sequence identity with any one of SEQUENCES: A1-A12; and
(iii) a VL region comprising a CDR1 comprising a sequence having at least 80% sequence identity with any one of SEQ ID NOs: 50-61.

In one embodiment, the antibody or fragment thereof comprises (or consists of) a VH region comprising a CDR3 comprising a sequence having at least 80% sequence identity with any one of SEQ ID NOs: 2, 3, 4, 5 or 6, such as 2, 3, 4 or 5, in particular 2, 3 or 4. In one embodiment, the antibody or fragment thereof comprises (or consists of) a VH region comprising a CDR2 comprising a sequence having at least 80% sequence identity with any one of SEQ ID NOs: 26, 27, 28, 29 or 30, such as 26, 27, 28 or 29, in particular 26, 27 or 28.

In one embodiment, the antibody or fragment thereof comprises (or consists of) a VH region comprising a CDR1 comprising a sequence having at least 80% sequence identity with any one of SEQ ID NOs:38, 39, 40, 41 or 42, such as 38, 39, 40 or 41, in particular 38, 39 or 40.

In one embodiment, the antibody or fragment thereof comprises (or consists of) a VH region comprising a CDR3 comprising a sequence having at least 80% sequence identity with any one of SEQ ID NOs: 8, 9, 10 or 11. In one embodiment, the antibody or fragment thereof comprises (or consists of) a VH region comprising a CDR2 comprising a sequence having at least 80% sequence identity with any one of SEQ ID NOs: 32, 33, 34 or 35. In one embodiment, the antibody or fragment thereof comprises (or consists of) a VH region comprising a CDR1 comprising a sequence having at least 80% sequence identity with any one of SEQ ID NOs: 44, 45, 46 or 47.

In one embodiment, the VH region comprises a CDR3 comprising a sequence of SEQ ID NO: 2, a CDR2 comprising a sequence of SEQ ID NO: 26, and a CDR1 comprising a sequence of SEQ ID NO: 38. In one embodiment, the CDR3 consists of a sequence of SEQ ID NO: 2, the CDR2 consists of a sequence of SEQ ID NO: 26, and the CDR1 consists of a sequence of SEQ ID NO: 38.

In one embodiment, the VH region comprises a CDR3 comprising a sequence of SEQ ID NO: 3, a CDR2 comprising a sequence of SEQ ID NO: 27, and a CDR1 comprising a sequence of SEQ ID NO: 39. In one embodiment, the CDR3 consists of a sequence of SEQ ID NO: 3, the CDR2 consists of a sequence of SEQ ID NO: 27, and the CDR1 consists of a sequence of SEQ ID NO: 39.

In one embodiment, the VH region comprises a CDR3 comprising a sequence of SEQ ID NO: 4, a CDR2 comprising a sequence of SEQ ID NO: 28, and a CDR1 comprising a sequence of SEQ ID NO: 40. In one embodiment, the CDR3 consists of a sequence of SEQ ID NO: 4, the CDR2 consists of a sequence of SEQ ID NO: 28, and the CDR1 consists of a sequence of SEQ ID NO: 40.

In one embodiment, the VH region comprises a CDR3 comprising a sequence of SEQ ID NO: 5, a CDR2 comprising a sequence of SEQ ID NO: 29, and a CDR1 comprising a sequence of SEQ ID NO: 41. In one embodiment, the CDR3 consists of a sequence of SEQ ID NO: 5, the CDR2 consists of a sequence of SEQ ID NO: 29, and the CDR1 consists of a sequence of SEQ ID NO: 41.

In one embodiment, the VH region comprises a CDR3 comprising a sequence of SEQ ID NO: 6, a CDR2 comprising a sequence of SEQ ID NO: 30, and a CDR1 comprising a sequence of SEQ ID NO: 42. In one embodiment, the CDR3 consists of a sequence of SEQ ID NO: 6, the CDR2 consists of a sequence of SEQ ID NO: 30, and the CDR1 consists of a sequence of SEQ ID NO: 42.

In one embodiment, the VH region comprises a CDR3 comprising a sequence of SEQ ID NO: 8, a CDR2 comprising a sequence of SEQ ID NO: 32, and a CDR1 comprising a sequence of SEQ ID NO: 44. In one embodiment, the CDR3 consists of a sequence of SEQ ID NO: 8, the CDR2 consists of a sequence of SEQ ID NO: 32, and the CDR1 consists of a sequence of SEQ ID NO: 44.

In one embodiment, the VH region comprises a CDR3 comprising a sequence of SEQ ID NO: 9, a CDR2 comprising a sequence of SEQ ID NO: 33, and a CDR1 comprising a sequence of SEQ ID NO: 45. In one embodiment, the CDR3 consists of a sequence of SEQ ID NO: 9, the CDR2 consists of a sequence of SEQ ID NO: 33, and the CDR1 consists of a sequence of SEQ ID NO: 45.

In one embodiment, the VH region comprises a CDR3 comprising a sequence of SEQ ID NO: 10, a CDR2 sequence of SEQ ID NO: 34, and a CDR1 sequence of SEQ ID NO: 46.

In one embodiment, the CDR3 consists of a sequence of SEQ ID NO: 10, the CDR2 consists of a sequence of SEQ ID NO: 34, and the CDR1 consists of a sequence of SEQ ID NO: 46.

In one embodiment, the VH region comprises a CDR3 comprising a sequence of SEQ ID NO: 11, a CDR2 sequence of SEQ ID NO: 35, and a CDR1 sequence of SEQ ID NO: 47.

In one embodiment, the CDR3 consists of a sequence of SEQ ID NO: 11, the CDR2 consists of a sequence of SEQ ID NO: 35, and the CDR1 consists of a sequence of SEQ ID NO: 47.

In one embodiment, the antibody or fragment thereof comprises (or consists of) a VL region comprising a CDR3 comprising a sequence having at least 80% sequence identity with any one of SEQ ID NOs: 14-25, such as SEQ ID NOs: 14, 15, 16, 17 or 18 such as 14, 15, 16 or 17, in particular 14, 15 or 16. In one embodiment, the antibody or fragment thereof comprises (or consists of) a VL region comprising a CDR2 comprising a sequence having at least 80% sequence identity with any one of SEQUENCES: A1-A12 (of Table 2), such as SEQUENCES: A1, A2, A3, A4 or A5, such as A1, A2, A3 or A4, in particular A1, A2 or A3. In one embodiment, the antibody or fragment thereof comprises (or consists of) a VL region comprising a CDR1 comprising a sequence having at least 80% sequence identity with any one of SEQ ID NOs: 50-81, such as SEQ ID NOs: 50, 51, 52, 53 or 54, such as 50, 51, 52 or 53, in particular 50, 51 or 52.

In one embodiment, the VL region comprises a CDR3 comprising a sequence of SEQ ID NO: 14, a CDR2 comprising a sequence of SEQUENCE: A1, and a CDR1 comprising a sequence of SEQ ID NO: 50. In one embodiment, the CDR3 consists of a sequence of SEQ ID NO: 14, the CDR2 consists of a sequence of SEQUENCE: A1, and the CDR1 consists of a sequence of SEQ ID NO: 50.

In one embodiment, the VL region comprises a CDR3 comprising a sequence of SEQ ID NO: 15, a CDR2 comprising a sequence of SEQUENCE: A2, and a CDR1 comprising a sequence of SEQ ID NO: 51. In one embodiment, the CDR3 consists of a sequence of SEQ ID NO: 15, the CDR2 consists of a sequence of SEQUENCE: A2, and the CDR1 consists of a sequence of SEQ ID NO: 51.

In one embodiment, the VL region comprises a CDR3 comprising a sequence of SEQ ID NO: 16, a CDR2 comprising a sequence of SEQUENCE: A3, and a CDR1 comprising a sequence of SEQ ID NO: 52. In one embodiment, the CDR3 consists of a sequence of SEQ ID NO: 16, the CDR2 consists of a sequence of SEQUENCE: A3, and the CDR1 consists of a sequence of SEQ ID NO: 52.

In one embodiment, the VL region comprises a CDR3 comprising a sequence of SEQ ID NO: 17, a CDR2 comprising a sequence of SEQUENCE: A4, and a CDR1 comprising a sequence of SEQ ID NO: 53. In one embodiment, the CDR3 consists of a sequence of SEQ ID NO: 17, the CDR2 consists of a sequence of SEQUENCE: A4, and the CDR1 consists of a sequence of SEQ ID NO: 53.

In one embodiment, the VL region comprises a CDR3 comprising a sequence of SEQ ID NO: 18, a CDR2 comprising a sequence of SEQUENCE: A5, and a CDR1 comprising a sequence of SEQ ID NO: 54. In one embodiment, the CDR3 consists of a sequence of SEQ ID NO: 18, the CDR2 consists of a sequence of SEQUENCE: A5, and the CDR1 consists of a sequence of SEQ ID NO: 54.

In one embodiment, the VL region comprises a CDR3 comprising a sequence of SEQ ID NO: 20, a CDR2 comprising a sequence of SEQUENCE: A7, and a CDR1 comprising a sequence of SEQ ID NO: 56. In one embodiment, the CDR3 consists of a sequence of SEQ ID NO: 20, the CDR2 consists of a sequence of SEQUENCE: A7, and the CDR1 consists of a sequence of SEQ ID NO: 56.

In one embodiment, the VL region comprises a CDR3 comprising a sequence of SEQ ID NO: 21, a CDR2 comprising a sequence of SEQUENCE: A8, and a CDR1 comprising a sequence of SEQ ID NO: 57. In one embodiment, the CDR3 consists of a sequence of SEQ ID NO: 21, the CDR2 consists of a sequence of SEQUENCE: A8, and the CDR1 consists of a sequence of SEQ ID NO: 57.

In one embodiment, the VL region comprises a CDR3 comprising a sequence of SEQ ID NO: 22, a CDR2 comprising a sequence of SEQUENCE: A9, and a CDR1 comprising a sequence of SEQ ID NO: 58. In one embodiment, the CDR3 consists of a sequence of SEQ ID NO: 22, the CDR2 consists of a sequence of SEQUENCE: A9, and the CDR1 consists of a sequence of SEQ ID NO: 58.

In one embodiment, the VL region comprises a CDR3 comprising a sequence of SEQ ID NO: 23, a CDR2 comprising a sequence of SEQUENCE: A10, and a CDR1 comprising a sequence of SEQ ID NO: 59. In one embodiment, the CDR3 consists of a sequence of SEQ ID NO: 23, the CDR2 consists of a sequence of SEQUENCE: A10, and the CDR1 consists of a sequence of SEQ ID NO: 59.

In one embodiment, the VH region comprises a CDR3 comprising a sequence of SEQ ID NO: 2, a CDR2 comprising a sequence of SEQ ID NO: 26, a CDR1 comprising a sequence of SEQ ID NO: 38, and the VL region comprises a CDR3 comprising a sequence of SEQ ID NO: 14, a CDR2 comprising a sequence of SEQUENCE: A1, and a CDR1 comprising a sequence of SEQ ID NO: 50. In one embodiment, the HCDR3 consists of a sequence of SEQ ID NO: 2, the HCDR2 consists of a sequence of SEQ ID NO: 26, the HCDR1 consists of a sequence of SEQ ID NO: 38, the LCDR3 consists of a sequence of SEQ ID NO: 14, the LCDR2 consists of a sequence of SEQUENCE: A1, and the LCDR1 consists of a sequence of SEQ ID NO: 50.

In one embodiment, the VH region comprises a CDR3 comprising a sequence of SEQ ID NO: 3, a CDR2 comprising a sequence of SEQ ID NO: 27, a CDR1 comprising a sequence of SEQ ID NO: 39, and the VL region comprises a CDR3 comprising a sequence of SEQ ID NO: 15, a CDR2 comprising a sequence of SEQUENCE: A2, and a CDR1 comprising a sequence of SEQ ID NO: 51. In one embodiment, the HCDR3 consists of a sequence of SEQ ID NO: 3, the HCDR2 consists of a sequence of SEQ ID NO: 27, the HCDR1 consists of a sequence of SEQ ID NO: 39, the LCDR3 consists of a sequence of SEQ ID NO: 15, the LCDR2 consists of a sequence of SEQUENCE: A2, and the LCDR1 consists of a sequence of SEQ ID NO: 51.

In one embodiment, the VH region comprises a CDR3 comprising a sequence of SEQ ID NO: 4, a CDR2 comprising a sequence of SEQ ID NO: 28, a CDR1 comprising a sequence of SEQ ID NO: 40, and the VL region comprises a CDR3 comprising a sequence of SEQ ID NO: 16, a CDR2 comprising a sequence of SEQUENCE: A3, and a CDR1 comprising a sequence of SEQ ID NO: 52. In one embodiment, the HCDR3 consists of a sequence of SEQ ID NO: 4, the HCDR2 consists of a sequence of SEQ ID NO: 28, the HCDR1 consists of a sequence of SEQ ID NO: 40, the LCDR3 consists of a sequence of SEQ ID NO: 16, the LCDR2 consists of a sequence of SEQUENCE: A3, and the LCDR1 consists of a sequence of SEQ ID NO: 52.

In one embodiment, the VH region comprises a CDR3 comprising a sequence of SEQ ID NO: 5, a CDR2 comprising a sequence of SEQ ID NO: 29, a CDR1 comprising a sequence of SEQ ID NO: 41, and the VL region comprises a CDR3 comprising a sequence of SEQ ID NO: 17, a CDR2 comprising a sequence of SEQUENCE: A4, and a CDR1 comprising a sequence of SEQ ID NO: 53. In one embodiment, the HCDR3 consists of a sequence of SEQ ID NO: 5, the HCDR2 consists of a sequence of SEQ ID NO: 29, the HCDR1 consists of a sequence of SEQ ID NO: 41, the LCDR3 consists of a sequence of SEQ ID NO: 17, the LCDR2 consists of a sequence of SEQUENCE: A4, and the LCDR1 consists of a sequence of SEQ ID NO: 53.

In one embodiment, the VH region comprises a CDR3 comprising a sequence of SEQ ID NO: 6, a CDR2 comprising a sequence of SEQ ID NO: 30, a CDR1 comprising a sequence of SEQ ID NO: 42, and the VL region comprises a CDR3 comprising a sequence of SEQ ID NO: 18, a CDR2 comprising a sequence of SEQUENCE: A5, and a CDR1 comprising a sequence of SEQ ID NO: 54. In one embodiment, the HCDR3 consists of a sequence of SEQ ID NO: 6, the HCDR2 consists of a sequence of SEQ ID NO: 30, the HCDR1 consists of a sequence of SEQ ID NO: 42, the LCDR3 consists of a sequence of SEQ ID NO: 18, the LCDR2 consists of a sequence of SEQUENCE: A5, and the LCDR1 consists of a sequence of SEQ ID NO: 54.

In one embodiment, the VH region comprises a CDR3 comprising a sequence of SEQ ID NO: 7, a CDR2 comprising a sequence of SEQ ID NO: 31, a CDR1 comprising a sequence of SEQ ID NO: 43, and the VL region comprises a CDR3 comprising a sequence of SEQ ID NO: 19, a CDR2 comprising a sequence of SEQUENCE: A6, and a CDR1 comprising a sequence of SEQ ID NO: 55. In one embodiment, the HCDR3 consists of a sequence of SEQ ID NO: 7, the HCDR2 consists of a sequence of SEQ ID NO: 31, the HCDR1 consists of a sequence of SEQ ID NO: 43, the LCDR3 consists of a sequence of SEQ ID NO: 19, the LCDR2 consists of a sequence of SEQUENCE: A6, and the LCDR1 consists of a sequence of SEQ ID NO: 55.

In one embodiment, the VH region comprises a CDR3 comprising a sequence of SEQ ID NO: 8, a CDR2 comprising a sequence of SEQ ID NO: 32, a CDR1 comprising a sequence of SEQ ID NO: 44, and the VL region comprises a CDR3 comprising a sequence of SEQ ID NO: 20, a CDR2 comprising a sequence of SEQUENCE: A7, and a CDR1 comprising a sequence of SEQ ID NO: 56. In one embodiment, the HCDR3 consists of a sequence of SEQ ID NO: 8, the HCDR2 consists of a sequence of SEQ ID NO: 32, the HCDR1 consists of a sequence of SEQ ID NO: 44, the LCDR3 consists of a sequence of SEQ ID NO: 20, the LCDR2 consists of a sequence of SEQUENCE: A7, and the LCDR1 consists of a sequence of SEQ ID NO: 58.

In one embodiment, the VH region comprises a CDR3 comprising a sequence of SEQ ID NO: 9, a CDR2 comprising a sequence of SEQ ID NO: 33, a CDR1 comprising a sequence of SEQ ID NO: 45, and the VL region comprises a CDR3 comprising a sequence of SEQ ID NO: 21, a CDR2 comprising a sequence of SEQUENCE: A8, and a CDR1 comprising a sequence of SEQ ID NO: 57. In one embodiment, the HCDR3 consists of a sequence of SEQ ID NO: 9, the HCDR2 consists of a sequence of SEQ ID NO: 33, the HCDR1 consists of a sequence of SEQ ID NO: 45, the LCDR3 consists of a sequence of SEQ ID NO: 21, the LCDR2 consists of a sequence of SEQUENCE: A8, and the LCDR1 consists of a sequence of SEQ ID NO: 57.

In one embodiment, the VH region comprises a CDR3 comprising a sequence of SEQ ID NO: 10, a CDR2 comprising a sequence of SEQ ID NO: 34, a CDR1 comprising a sequence of SEQ ID NO: 46, and the VL region comprises a CDR3 comprising a sequence of SEQ ID NO: 22, a CDR2 comprising a sequence of SEQUENCE: A9, and a CDR1 comprising a sequence of SEQ ID NO: 58. In one embodiment, the HCDR3 consists of a sequence of SEQ ID NO: 10, the HCDR2 consists of a sequence of SEQ ID NO: 34, the HCDR1 consists of a sequence of SEQ ID NO: 46, the LCDR3 consists of a sequence of SEQ ID NO: 22, the LCDR2 consists of a sequence of SEQUENCE: A9, and the LCDR1 consists of a sequence of SEQ ID NO: 58.

In one embodiment, the VH region comprises a CDR3 comprising a sequence of SEQ ID NO: 11, a CDR2 comprising a sequence of SEQ ID NO: 35, a CDR1 comprising a sequence of SEQ ID NO: 47, and the VL region comprises a CDR3 comprising a sequence of SEQ ID NO: 23, a CDR2 comprising a sequence of SEQUENCE: A10, and a CDR1 comprising a sequence of SEQ ID NO: 59. In one embodiment, the HCDR3 consists of a sequence of SEQ ID NO: 11, the HCDR2 consists of a sequence of SEQ ID NO: 35, the HCDR1 consists of a sequence of SEQ ID NO: 47, the LCDR3 consists of a sequence of SEQ ID NO: 23, the LCDR2 consists of a sequence of SEQUENCE: A10, and the LCDR1 consists of a sequence of SEQ ID NO: 59.

In one embodiment, the VH region comprises a CDR3 comprising a sequence of SEQ ID NO: 12, a CDR2 comprising a sequence of SEQ ID NO: 38, a CDR1 comprising a sequence of SEQ ID NO: 48, and the VL region comprises a CDR3 comprising a sequence of SEQ ID NO: 24, a CDR2 comprising a sequence of SEQUENCE: All, and a CDR1 comprising a sequence of SEQ ID NO: 60. In one embodiment, the HCDR3 consists of a sequence of SEQ ID NO: 12, the HCDR2 consists of a sequence of SEQ ID NO: 36, the HCDR1 consists of a sequence of SEQ ID NO: 48, the LCDR3 consists of a sequence of SEQ ID NO: 24, the LCDR2 consists of a sequence of SEQUENCE: A11, and the LCDR1 consists of a sequence of SEQ ID NO: 60.

In one embodiment, the VH region comprises a CDR3 comprising a sequence of SEQ ID NO: 13, a CDR2 comprising a sequence of SEQ ID NO: 37, a CDR1 comprising a sequence of SEQ ID NO: 49, and the VL region comprises a CDR3 comprising a sequence of SEQ ID NO: 25, a CDR2 comprising a sequence of SEQUENCE: A12, and a CDR1 comprising a sequence of SEQ ID NO: 61. In one embodiment, the HCDR3 consists of a sequence of SEQ ID NO: 13, the HCDR2 consists of a sequence of SEQ ID NO: 37, the HCDR1 consists of a sequence of SEQ ID NO: 49, the LCDR3 consists of a sequence of SEQ ID NO: 25, the LCDR2 consists of a sequence of SEQUENCE: A12, and the LCDR1 consists of a sequence of SEQ ID NO: 61.

In one embodiment, the antibody or fragment thereof comprises one or more CDR sequences as described in Table 2. In a further embodiment, the antibody or fragment thereof comprises one or more (such as all) CDR sequences of clone 1252_P01_C08 as described in Table 2. In an alternative embodiment, the antibody or fragment thereof comprises one or more (such as all) CDR sequences of clone 1245_P01_E07 as described in Table 2. In an alternative embodiment, the antibody or fragment thereof comprises one or more (such as all) CDR sequences of clone 1245_P02_G04 as described in Table 2. In an alternative embodiment, the antibody or fragment thereof comprises one or more (such as all) CDR sequences of clone 1245_P02_B07 as described in Table 2. In an alternative embodiment, the antibody or fragment thereof comprises one or more (such as all) CDR sequences of clone 1251_P02_C05 as described in Table 2. In an alternative embodiment, the antibody or fragment thereof comprises one or more (such as all) CDR sequences of clone 1139_P01_E04 as described in Table 2. In an alternative embodiment, the antibody or fragment thereof comprises one or more (such as all) CDR sequences of clone 1245_P02_F07 as described in Table 2. In an alternative embodiment, the antibody or fragment thereof comprises one or more (such as all) CDR sequences of clone 1245_P01_G06 as described in Table 2. In an alternative embodiment, the antibody or fragment thereof comprises one or more (such as all) CDR sequences of clone 1245_P01_G09 as described in Table 2. In an alternative embodiment, the antibody or fragment thereof comprises one or more (such as all) CDR sequences of clone 1138_P01_B09 as described in Table 2. In an alternative embodiment, the antibody or fragment thereof comprises one or more (such as all) CDR sequences of clone 1251_P02_G10 as described in Table 2.

TABLE 2
Example anti-TCR delta variable 1 (anti-Vo1) antibodies

| Clone | Heavy | SEQ | Heavy | SEQ | Heavy | SEQ | Light | SEQ | Light | | Light | SEQ |
|---|---|---|---|---|---|---|---|---|---|---|---|---|
| ID | CDR1 | ID NO. | CDR2 | ID NO. | CDR3 | ID NO. | CDR1 | ID NO. | CDR2 | SEQ | CDR3 | ID NO. |
| 1245_ | GFTFS | 38 | ISSSG | 26 | VDYAD | 2 | QSIGT | 50 | VAS | A1 | QQSYS | 14 |
| P01_ | DYY | | STI | | AFDI | | Y | | | | TLLT | |
| E07 | | | | | | | | | | | | |
| 1252_ | GFTVS | 39 | IYSGG | 27 | PIELG | 3 | NIGSQ | 51 | YDS | A2 | QVWDS | 15 |
| P01_ | SNY | | ST | | AFDI | | S | | | | SSDHV | |
| C08 | | | | | | | | | | | V | |
| 1245_ | GDSVS | 40 | TYYRS | 28 | TWSGY | 4 | QDIND | 52 | DAS | A3 | QQSYS | 16 |
| P02_ | SKSAA | | KWST | | VDV | | W | | | | TPQVT | |
| G04 | | | | | | | | | | | | |
| 1245_ | GFTFS | 41 | ISSSG | 29 | ENYLN | 5 | QSLSN | 53 | AAS | A4 | QQSYS | 17 |
| P01_ | DYY | | STI | | AFDI | | Y | | | | TPLT | |
| B07 | | | | | | | | | | | | |
| 1251_ | GFTFS | 42 | ISGGG | 30 | DSGVA | 6 | QNIRT | 54 | DAS | A5 | QQFKR | 18 |
| P02_ | SYA | | GTT | | FDI | | W | | | | YPPT | |
| C05 | | | | | | | | | | | | |
| 1141_ | GYSFT | 43 | IYPGD | 31 | HQVDT | 7 | RSDVG | 55 | EVS | A6 | SSYTS | 19 |
| P01_ | SYW | | SDT | | RTADY | | GYNY | | | | TSTLV | |
| E01 | | | | | | | | | | | | |
| 1139_ | GDSVS | 44 | TYYRS | 32 | SWNDA | 8 | QSIST | 56 | DAS | A7 | QQSYS | 20 |
| P01_ | SNSAA | | KWYN | | FDI | | W | | | | TPLT | |
| E04 | | | | | | | | | | | | |
| 1245_ | GDSVS | 45 | TYYRS | 33 | DYYYS | 9 | QSISS | 57 | DAS | A8 | QQSHS | 21 |
| P02_ | SNSAA | | KWYN | | MDV | | W | | | | HPPT | |
| F07 | | | | | | | | | | | | |
| 1245_ | GFTFS | 46 | ISSSG | 34 | HSWND | 10 | QSISS | 58 | AAS | A9 | QQSYS | 22 |
| P01_ | DYY | | STI | | AFDV | | Y | | | | TPDT | |
| G06 | | | | | | | | | | | | |
| 1245_ | GDSVS | 47 | TYYGS | 135 | DYYYS | 11 | QSIST | 59 | DAS | A10 | QQSYS | 23 |
| P01_ | SNSAA | | KWYN | | MDV | | W | | | | TPVT | |
| G09 | | | | | | | | | | | | |
| 1138_ | GFTFS | 48 | ISSSG | 36 | HSWSD | 12 | QDISN | 60 | DAS | A11 | QQSYS | 24 |
| P01_ | DYY | | STI | | AFDI | | Y | | | | TPLT | |
| B09 | | | | | | | | | | | | |
| 1251_ | GFTFS | 49 | ISSSG | 37 | HSWND | 13 | QSISS | 61 | AAS | A12 | QQSYS | 25 |
| P02_ | DYY | | STI | | AFDI | | H | | | | TLLT | |
| G10 | | | | | | | | | | | | |

Suitably the VH and VL regions recited above each comprise four framework regions (FR1-FR4). In one embodiment, the antibody or fragment thereof comprises a framework region (e.g. FR1, FR2, FR3 and/or FR4) comprising a sequence having at least 80% sequence identity with the framework region in any one of SEQ ID NOs: 62-85. In one embodiment, the antibody or fragment thereof comprises a framework region (e.g. FR1, FR2, FR3 and/or FR4) comprising a sequence having at least 90%, such as at least 95%, 97% or 99% sequence identity with the framework region in any one of SEQ ID NOs: 62-85. In one embodiment, the antibody or fragment thereof comprises a framework region (e.g. FR1, FR2, FR3 and/or FR4) comprising a sequence in any one of SEQ ID NOs: 62-85. In one embodiment, the antibody or fragment thereof comprises a framework region (e.g. FR1, FR2, FR3 and/or FR4) consisting of a sequence in any one of SEQ ID NOs: 62-85.

The antibodies described herein may be defined by their full light chain and/or heavy chain variable sequences. In one embodiment the antibody or fragment thereof comprises an amino acid sequence having at least 80% sequence identity with any one of SEQ ID NOs: 62-85. In one embodiment the antibody or fragment thereof consists of an amino acid sequence having at least 80% sequence identity with any one of SEQ ID NOs: 62-85.

In one embodiment, the antibody or fragment thereof comprises a VH region comprising an amino acid sequence having at least 80% sequence identity with any one of SEQ ID NOs: 62-73. In one embodiment, the antibody or fragment thereof comprises a VH region consisting of an amino acid sequence having at least 80% sequence identity with any one of SEQ ID NOs: 62-73. In a further embodiment, the VH region comprises an amino acid sequence having at least 80% sequence identity with any one of SEQ ID NOs: 62, 63, 64, 65 or 66, such as 62, 63, 64 or 65, in particular 62, 63 or 64. In a further embodiment, the VH region consists of an amino acid sequence having at least 80% sequence identity with any one of SEQ ID NOs: 62, 63, 64, 65 or 66, such as 62, 63, 64 or 65, in particular 62, 63 or 64. In a further embodiment, the VH region comprises an amino acid sequence having at least 80% sequence identity with any one of SEQ ID NOs: 68, 69, 70, 71, 72 or 73, such as 68, 69, 70 or 71. In a further embodiment, the VH region consists of an amino acid sequence having at least 80% sequence identity with any one of SEQ ID NOs: 68, 69, 70, 71, 72 or 73, such as 68, 69, 70 or 71.

In one embodiment, the antibody or fragment thereof comprises a VL region comprising an amino acid sequence having at least 80% sequence identity with any one of SEQ ID NOs: 74-85. In one embodiment, the antibody or fragment thereof comprises a VL region consisting of an amino acid sequence having at least 80% sequence identity with any one of SEQ ID NOs: 74-85. In a further embodiment, the VL region comprises an amino acid sequence having at least 80% sequence identity with any one of SEQ ID NOs: 74, 75, 76, 77 or 78, such as 74, 75, 76 or 77, in particular 74, 75, or 76. In a further embodiment, the VL region consists of an amino acid sequence having at least 80% sequence identity with any one of SEQ ID NOs: 74, 75, 76, 77 or 78, such as 74, 75, 76 or 77, in particular 74, 75, or 76.

In a further embodiment, the VL region comprises an amino acid sequence having at least 80% sequence identity with any one of SEQ ID NOs: 80, 81, 82, 83, 84 or 85, such as 80, 81, 82 or 83. In a further embodiment, the VL region consists of an amino acid sequence having at least 80% sequence identity with any one of SEQ ID NOs: 80, 81, 82, 83, 84 or 85, such as 80, 81, 82 or 83.

In a further embodiment, the antibody or fragment thereof comprises a VH region comprising an amino acid sequence having at least 80% sequence identity with any one of SEQ ID NOs: 62-73 and a VL region comprising an amino acid sequence having at least 80% sequence identity with any one of SEQ ID NOs: 74-85. In a further embodiment, the antibody or fragment thereof comprises a VH region consisting of an amino acid sequence having at least 80% sequence identity with any one of SEQ ID NOs: 62-73 and a VL region consisting of an amino acid sequence having at least 80% sequence identity with any one of SEQ ID NOs:74-85.

In one embodiment, the antibody or fragment thereof comprises a VH region comprising an amino acid sequence of SEQ ID NO: 63 (1252_P01_C08). In an alternative embodiment, the antibody or fragment thereof comprises a VH region comprising an amino acid sequence of SEQ ID NO: 62 (1245P01_E07). In an alternative embodiment, the antibody or fragment thereof comprises a VH region comprising an amino acid sequence of SEQ ID NO: 64 (1245_P02_G04). In an alternative embodiment, the antibody or fragment thereof comprises a VH region comprising an amino acid sequence of SEQ ID NO: 68 (1139_P01_E04). In an alternative embodiment, the antibody or fragment thereof comprises a VH region comprising an amino acid sequence of SEQ ID NO: 69 (1245_P02_F07). In an alternative embodiment, the antibody or fragment thereof comprises a VH region comprising an amino acid sequence of SEQ ID NO: 70 (1245_P01_G06). In an alternative embodiment, the antibody or fragment thereof comprises a VH region comprising an amino acid sequence of SEQ ID NO: 71 (1245_P01_G09).

In one embodiment, the antibody or fragment thereof comprises a VH region consisting of an amino acid sequence of SEQ ID NO: 63 (1252_P01_C08). In an alternative embodiment, the antibody or fragment thereof comprises a VH region consisting of an amino acid sequence of SEQ ID NO: 62 (1245_P01_E07). In an alternative embodiment, the antibody or fragment thereof comprises a VH region consisting of an amino acid sequence of SEQ ID NO: 64 (1245_P02_G04). In an alternative embodiment, the antibody or fragment thereof comprises a VH region consisting of an amino acid sequence of SEQ ID NO: 68 (1139_P01_E04). In an alternative embodiment, the antibody or fragment thereof comprises a VH region consisting of an amino acid sequence of SEQ ID NO: 69 (1245P02_F07). In an alternative embodiment, the antibody or fragment thereof comprises a VH region consisting of an amino acid sequence of SEQ ID NO: 70 (1245_P01_G06). In an alternative embodiment, the antibody or fragment thereof comprises a VH region consisting of an amino acid sequence of SEQ ID NO: 71 (1245_P01_G09).

In one embodiment, the antibody or fragment thereof comprises a VL region comprising an amino acid sequence of SEQ ID NO: 75 (1252_P01_C08). In an alternative embodiment, the antibody or fragment thereof comprises a VL region comprising an amino acid sequence of SEQ ID NO: 74 (1245P01_E07). In an alternative embodiment, the antibody or fragment thereof comprises a VL region comprising an amino acid sequence of SEQ ID NO: 76 (1245_P02_G04). In an alternative embodiment, the antibody or fragment thereof comprises a VL region comprising an amino acid sequence of SEQ ID NO: 80 (1139_P01_E04). In an alternative embodiment, the antibody or fragment thereof comprises a VL region comprising an amino acid sequence of SEQ ID NO: 81 (1245_P02F07). In an alternative embodiment, the antibody or fragment thereof comprises a VL region comprising an amino acid sequence of SEQ ID NO: 82 (1245_P01_G06). In an alternative embodiment, the antibody or fragment thereof comprises a VL region comprising an amino acid sequence of SEQ ID NO: 83 (1245_P01_G09).

In one embodiment, the antibody or fragment thereof comprises a VL region consisting of an amino acid sequence of SEQ ID NO: 75 (1252_P01_C08). In an alternative embodiment, the antibody or fragment thereof comprises a VL region consisting of an amino acid sequence of SEQ ID NO: 74 (1245_P01_E07). In an alternative embodiment, the antibody or fragment thereof comprises a VL region consisting of an amino acid sequence of SEQ ID NO: 76 (1245_P02_G04). In an alternative embodiment, the antibody or fragment thereof comprises a VL region consisting of an amino acid sequence of SEQ ID NO: 80 (1139_P01_E04). In an alternative embodiment, the antibody or fragment thereof comprises a VL region consisting of an amino acid sequence of SEQ ID NO: 81 (1245_P02_F07). In an alternative embodiment, the antibody or fragment thereof comprises a VL region consisting of an amino acid sequence of SEQ ID NO: 82 (1245_P01_G06). In an alternative embodiment, the antibody or fragment thereof comprises a VL region consisting of an amino acid sequence of SEQ ID NO: 83 (1245_P01_G09).

In one embodiment, the antibody or fragment thereof comprises a VH region comprising an amino acid sequence of SEQ ID NO: 63 (1252_P01_C08) and a VL region comprising an amino acid sequence of SEQ ID NO: 75 (1252_1P01_C08). In an alternative embodiment, the antibody or fragment thereof comprises a VH region comprising an amino acid sequence of SEQ ID NO: 62 (1245_P01_E07) and a VL region comprising an amino acid sequence of SEQ ID NO: 74 (1245_P01_E07). In an alternative embodiment, the antibody or fragment thereof comprises a VH region comprising an amino acid sequence of SEQ ID NO: 64 (1245_P02_G04) and a VL region comprising an amino acid sequence of SEQ ID NO: 76 (1245_P02_G04). In an alternative embodiment, the antibody or fragment thereof comprises a VH region comprising an amino acid sequence of SEQ ID NO: 68 (1139_P01_E04) and a VL region comprising an amino acid sequence of SEQ ID NO: 80 (1139_P01_E04). In an alternative embodiment, the antibody or fragment thereof comprises a VH region comprising an amino acid sequence of SEQ ID NO: 69 (1245_P02_F07) and a VL region comprising an amino acid sequence of SEQ ID NO: 81 (1245_P02_F07). In an alternative embodiment, the antibody or fragment thereof comprises a VH region comprising an amino acid sequence of SEQ ID NO: 70 (1245_P01_G06) and a VL region comprising an amino acid sequence of SEQ ID NO: 82 (1245_P01_G06). In an alternative embodiment, the antibody or fragment thereof comprises a VH region comprising an amino acid sequence of SEQ ID NO: 71 (1245_P01_G06) and a VL region comprising an amino acid sequence of SEQ ID NO: 83 (1245_P01_G09).

In one embodiment, the antibody or fragment thereof comprises a VH region consisting of an amino acid sequence of SEQ ID NO: 63 (1252_1P01_C08) and a VL region consisting of an amino acid sequence of SEQ ID NO: 75 (1252_P01_C08). In an alternative embodiment, the antibody or fragment thereof comprises a VH region consisting of an amino acid sequence of SEQ ID NO: 62 (1245_P01_E07) and a VL region consisting of an amino acid sequence of SEQ ID NO: 74 (1245_P01_E07). In an alternative embodiment, the antibody or fragment thereof comprises a VH region consisting of an amino acid sequence of SEQ ID NO: 64 (1245_P02_G04) and a VL region consisting of an amino acid sequence of SEQ ID NO: 76 (1245_P02_G04). In an alternative embodiment, the antibody or fragment thereof comprises a VH region consisting of an amino acid sequence of SEQ ID NO: 68 (1139_P01_E04) and a VL region consisting of an amino acid sequence of SEQ ID NO: 80 (1139_P01_E04). In an alternative embodiment, the antibody or fragment thereof comprises a VH region consisting of an amino acid sequence of SEQ ID NO: 69 (1245_P02_F07) and a VL region consisting of an amino acid sequence of SEQ ID NO: 81 (1245_P02_F07). In an alternative embodiment, the antibody or fragment thereof comprises a VH region consisting of an amino acid sequence of SEQ ID NO: 70 (1245_P01_G06) and a VL region consisting of an amino acid sequence of SEQ ID NO: 82 (1245_P01_G06). In an alternative embodiment, the antibody or fragment thereof comprises a VH region consisting of an amino acid sequence of SEQ ID NO: 71 (1245_P01_G09) and a VL region consisting of an amino acid sequence of SEQ ID NO: 83 (1245_P01_G09).

For fragments comprising both the VH and VL regions, these may be associated either covalently (e.g. via disulphide bonds or a linker) or non-covalently. The antibody fragment described herein may comprise an scFv, i.e. a fragment comprising a VH region and a VL region joined by a linker. In one embodiment, the VH and VL region are joined by a (e.g. synthetic) polypeptide linker. The polypeptide linker may comprise a (Gly₄Ser)ₙ linker, where n=from 1 to 8, e.g. 2, 3, 4, 5 or 7. The polypeptide linker may comprise a [(Gly₄Ser)ₙ(Gly₃AlaSer)ₘ]ₚ linker, where n=from 1 to 8, e.g. 2, 3, 4, 5 or 7, m=from 1 to 8, e.g. 0, 1, 2 or 3, and p=from 1 to 8, e.g. 1, 2 or 3. In a further embodiment, the linker comprises SEQ ID NO: 98. In a further embodiment, the linker consists of SEQ ID NO: 98.

In one embodiment, the antibody or fragment thereof comprises an amino acid sequence having at least 80% sequence identity with any one of SEQ ID NOs: 86-97. In a further embodiment, the antibody or fragment thereof comprises an amino acid sequence of any one of SEQ ID NOs: 86-97. In a yet further embodiment, the antibody or fragment thereof comprises an amino acid sequence of SEQ ID NO: 87 (1252_P01_C08). In an alternative embodiment, the antibody or fragment thereof comprises an amino acid sequence of SEQ ID NO: 86 (1245_P01_E07). In an alternative embodiment, the antibody or fragment thereof comprises an amino acid sequence of SEQ ID NO: 88 (1245_P02_G04). In an alternative embodiment, the antibody or fragment thereof comprises an amino acid sequence of SEQ ID NO: 92 (1139_P01_E04). In an alternative embodiment, the antibody or fragment thereof comprises an amino acid sequence of SEQ ID NO: 93 (1245_P02_F07). In an alternative embodiment, the antibody or fragment thereof comprises an amino acid sequence of SEQ ID NO: 94 (1245_P01_G06). In an alternative embodiment, the antibody or fragment thereof comprises an amino acid sequence of SEQ ID NO: 95 (1245_P01_G09).

In one embodiment, the antibody or fragment thereof consists of an amino acid sequence having at least 80% sequence identity with any one of SEQ ID NOs: 86-97. In a further embodiment, the antibody or fragment thereof consists of an amino acid sequence of any one of SEQ ID NOs: 86-97. In a yet further embodiment, the antibody or fragment thereof consists of an amino acid sequence of SEQ ID NO: 87 (1252_P01_C08). In an alternative embodiment, the antibody or fragment thereof consists of an amino acid sequence of SEQ ID NO: 86 (1245_P01_E07). In an alternative embodiment, the antibody or fragment thereof consists of an amino acid sequence of SEQ ID NO: 88 (1245_P02_G04). In an alternative embodiment, the antibody or fragment thereof consists of an amino acid sequence of SEQ ID NO: 92 (1139_P01_E04). In an alternative embodiment, the antibody or fragment thereof consists of an amino acid sequence of SEQ ID NO: 93 (1245_P02_F07). In an alternative embodiment, the antibody or fragment thereof consists of an amino acid sequence of SEQ ID NO: 94 (1245_P01_G06). In an alternative embodiment, the antibody or fragment thereof consists of an amino acid sequence of SEQ ID NO: 95 (1245_P01_G09).

It will be understood by a person skilled in the art that scFv constructs may be designed and made inclusive of N-terminal and C-terminal modifications to aid with translation, purification and detection. For example, at the N-terminus of an scFv sequence, an additional methionine and/or alanine amino acid residue may be included ahead of the canonical VH sequences (e.g. starting QVQ or EVQ). At the C-terminus (i.e. C-terminal to the canonical VL domain sequence ending as per the IMGT definition), additional sequences may be included such as (i) a partial sequence of the constant domain and/or (ii) additional synthetic sequences inclusive of tags, such as His-tags and Flag-tags, to aid with purification and detection. In one embodiment, SEQ ID NO: 124 is added to the C-terminus of any one of SEQ ID NOs: 86, 88-90, 92-97. In one embodiment, SEQ ID NO: 125 is added to the C-terminus of any one of SEQ ID NOs: 86, 88-90, 92-97. In one embodiment, SEQ ID NO: 126 is added to the C-terminus of any one of SEQ ID NOs: 87 or 91. In one embodiment, SEQ ID NO: 127 is added to the C-terminus of any one of SEQ ID NOs: 87 or 91. It is well understood that said scFv N- or C-terminal sequences are optional and can be removed, modified or substituted if alternate scFv design, translation, purification or detection strategies are adopted.

As described herein, the antibodies may be in any format. In a preferred embodiment, the antibody is in an IgG1 format. Therefore, in one embodiment, the antibody or fragment thereof comprises an amino acid sequence having at least 80% sequence identity with any one of SEQ ID NOs: 111-122. In a further embodiment, the antibody or fragment thereof comprises an amino acid sequence of any one of SEQ ID NOs: 111-122. In a yet further embodiment, the antibody or fragment thereof comprises an amino acid sequence of SEQ ID NOs: 111-116, such as SEQ ID NOs: 111-113 and 116. In a yet further embodiment, the antibody or fragment thereof comprises an amino acid sequence of SEQ ID NOs: 117-122, such as SEQ ID NOs: 117-120. In a yet further embodiment, the antibody or fragment thereof comprises an amino acid sequence of SEQ ID NOs: 111, 112, 116-120, such as SEQ ID NOs: 111, 112 or 116, or SEQ ID NOs: 117-120.

In one embodiment, the antibody or fragment thereof consists of an amino acid sequence having at least 80% sequence identity with any one of SEQ ID NOs: 111-122. In a further embodiment, the antibody or fragment thereof consists of an amino acid sequence of any one of SEQ ID NOs: 111-122. In a yet further embodiment, the antibody or fragment thereof consists of an amino acid sequence of SEQ ID NOs: 111-116, such as SEQ ID NOs: 111-113 and 116. In a yet further embodiment, the antibody or fragment thereof consists of an amino acid sequence of SEQ ID NOs: 117-122, such as SEQ ID NOs: 117-120. In a yet further embodiment, the antibody or fragment thereof consists of an amino acid sequence of SEQ ID NOs: 111, 112, 116-120, such as SEQ ID NOs: 111, 112 or 116, or SEQ ID NOs: 117-120.

In one embodiment, the antibody binds to the same, or essentially the same, epitope as, or competes with, an antibody or fragment thereof as defined herein. One can easily determine whether an antibody binds to the same epitope as, or competes for binding with, a reference anti-Vδ1 antibody by using routine methods known in the art. For example, to determine if a test antibody binds to the same epitope as a reference anti-Vδ1 antibody, the reference antibody is allowed to bind to a Vδ1 protein or peptide under saturating conditions. Next, the ability of a test antibody to bind to the Vδ1 chain is assessed. If the test antibody is able to bind to Vδ1 following saturation binding with the reference anti-Vδ1 antibody, it can be concluded that the test antibody binds to a different epitope than the reference anti-Vδ1 antibody. On the other hand, if the test antibody is not able to bind to the Vδ1 chain following saturation binding with the reference anti-Vδ1 antibody, then the test antibody may bind to the same epitope as the epitope bound by the reference anti-Vδ1 antibody.

The present invention also includes anti-Vδ1 antibodies that compete for binding to Vδ1 with an antibody or fragment thereof as defined herein, or an antibody having the CDR sequences of any of the exemplary antibodies described herein. For example, competitive assays can be performed with the antibody in order to determine what proteins, antibodies, and other antagonists compete for binding to the Vδ1 chain with the antibody and/or share the epitope. These assays are readily known to those of skill in the art; they evaluate competition between antagonists or ligands for a limited number of binding sites on a protein, e.g., Vδ1.

The antibody (or fragment thereof) is immobilized or insolubilized before or after the competition and the sample bound to the Vδ1 chain is separated from the unbound sample, for example, by decanting (where the antibody was pre-insolubilized) or by centrifuging (where the antibody was precipitated after the competitive reaction). Also, the competitive binding may be determined by whether the function is altered by the binding or lack of binding of the antibody to the protein, e.g. whether the antibody molecule inhibits or potentiates the enzymatic activity of, for example, a label. ELISA and other functional assays may be used, as known in the art and described herein.

Two antibodies bind to the same or overlapping epitope if each competitively inhibits (blocks) binding of the other to the target antigen. That is, a 1-, 5-, 10-, 20- or 100-fold excess of one antibody inhibits binding of the other by at least 50% but preferably 75%, 90% or even 99% as measured in a competitive binding assay. Alternatively, two antibodies have the same epitope if essentially all amino acid mutations in the target antigen that reduce or eliminate binding of one antibody reduce or eliminate binding of the other.

Additional routine experimentation (e.g. peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

In some embodiments, the antibody or fragment thereof contains a modified effector function through alteration to the sugars linked to Asn 297 (Kabat numbering scheme). In a further said modification, Asn 297 is not fucosylated or exhibits reduced fucosylation (i.e., a defucosylated antibody or a non-fucosylated antibody). Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. The antibody may be modified to prevent or inhibit fucosylation of the antibody. Typically, glycosylation modifications involve expressing said antibody or fragment thereof in a host cell containing alternate glycosylation processing capabilities either through targeted engineering or through targeted or serendipitous host or clone selection. These and other effector modifications are discussed further in recent reviews such as by Xinhua Wang et al. (2018) Protein & Cell 9: 63-73 and by Pereira et al. (2018) mAbs 10(5): 693-711 and which are hereby incorporated.

### Antibody Sequence Modifications

The antibodies and fragments thereof may be modified using known methods. Sequence modifications to antibody molecules described herein can be readily incorporate by those skilled in the art. The following examples are non-limiting.

During antibody discovery and sequence recovery from phage libraries, desired antibody variable domains may be re-formatted into full length IgG by sub-cloning. To accelerate the process, variable domains are often transferred using restriction enzymes. These unique restriction sites may introduce additional/alternate amino acids and away from the canonical sequence (such canonical sequences may be found, for example, in the international ImMunoGeneTics [VMGT] information system, see http://www.imgt.org). These may be introduced as kappa or lambda light chain sequence modifications.

### Kappa Light Chain Modifications

The variable kappa light chain variable sequences may be cloned using restriction sites (e.g. Nhe1-Not1) during re-formatting into full length IgG. More specifically, at the kappa light chain N-terminus, an additional Ala-Ser sequence was introduced to support cloning. Preferably, this additional AS sequence is then removed during further development such to generate the canonical N-terminal sequence. Hence, in one embodiment, kappa light chain containing antibodies described herein do not contain an AS sequence at their N-termini, i.e. SEQ ID NOs: 74, 78-78 and 80-85 do not comprise the initial AS sequence. In a further embodiment, SEQ ID NOs: 74 and 78-78 do not comprise the initial AS sequence. It will be understood that this embodiment also applies to other sequences included herein which contain this sequence (e.g. SEQ ID NOs: 86, 88-90 and 92-97).

Additional amino acid changes may be made to support cloning. For example, for the antibodies described herein, at the kappa light-chain variable-domain/constant domain border a valine-to-alanine change was introduced to support cloning. This resulted in a kappa constant domain modification. Specifically, this results in the constant domain beginning RTAAAPS (from a NotI restriction site). Preferably, this sequence can be modified during further development to generate the canonical kappa light-chain constant regions which start with RTVAAPS. Hence, in one embodiment kappa light chain containing antibodies described herein contain a constant domain stating with the sequence RTV. Therefore, in one embodiment, sequence RTAAAPS of SEQ ID NOs: 111-114 and 117-122 is replaced with sequence RTVAAPS.

### Lambda Light Chain Modifications

Similar to the kappa example above, the lambda light chain variable domains may also be cloned by introducing restriction sites (e.g. Nhe1-Not1) during re-formatting into full length IgG. More specifically, at the lambda light chain N-terminus, an additional Ala-Ser sequence may be introduced to support cloning. Preferably, this additional AS sequence is then removed during further development such to generate the canonical N-terminal sequence. Hence, in one embodiment, lambda light chain containing antibodies described herein do not contain an AS sequence at their N-termini i.e. SEQ ID NOs: 75 and 79 do not comprise the initial AS sequence. It will be understood that this embodiment also applies to other sequences included herein which contain this sequence (e.g. SEQ ID NOs: 87, 91, 115 and 116). In one embodiment, SEQ ID NO: 75 does not contain the initial six residues, i.e. the ASSYEL sequence is removed.

As another example, for the antibodies described herein at the lambda light-chain variable-domain/constant domain border a lysine-to-alanine sequence change was introduced to support cloning. This resulted in a lambda constant domain modification. Specifically, this results in the constant domain beginning with GQPAAAPS (from a NotI restriction site). Preferably, this sequence can be modified during further development such to generate the canonical lambda light constant region which starts GQPKAAPS. Hence, in one embodiment, lambda light chain containing antibodies described herein contain a constant domain starting with the sequence GQPK. Therefore, in one embodiment, sequence GQPAAAPS of SEQ ID NO: 115 or 116 is replaced with sequence GQPKAAPS.

### Heavy Chain Modifications

Typically, human variable heavy chain sequences start with either the basic glutamine (Q) or acidic glutamate (E). However, both such sequences are then known to convert to the acidic amino acid residue, pyro-glutamate (pE). The Q to pE conversion results in a charge change to the antibody, whilst an E to pE conversion does not change the charge of the antibody. Hence to avoid a variable charge-change over time one option is to modify a starting heavy chain sequence from Q to E in the first instance. Hence, in one embodiment, the heavy chain of antibody described herein contains a Q to E modification at the N-terminus. In particular, the initial residue of SEQ ID NOs: 62, 64 and/or 67-71 may be modified from Q to E. It will be understood that this embodiment also applies to other sequences included herein which contain this sequence (e.g. SEQ ID NOs: 86, 88, 91-97 and 111, 112, 115, 117-120).

Furthermore, the C-terminus of the IgG1 constant domain ends with PGK. However, the terminal basic lysine (K) is then often cleaved during expression (e.g. in CHO cells). This in turn results in charge change to the antibody through varied loss of the C-terminal lysine residue. Therefore, one option is to remove the lysine in the first instance resulting in a uniform and consistent heavy chain C-terminus sequence ending in PG. Hence, in one embodiment, the heavy chain of an antibody described herein has the terminal K removed from its C-terminus. In particular, the antibody of the invention may comprise any one of SEQ ID NOs: 111-122 where the terminal lysine residue has been removed.

### Optional Allotype Modifications

During antibody discovery, specific human allotypes may be employed. Optionally, the antibodies can be switched to differing human allotypes during development. By way of non-limiting example, for the kappa chain there are three human allotypes designated Km1, Km1,2 and Km3 which define three Km alleles (using allotype numbering): Km1 correlates with valine 153 (IMGT V45.1) and leucine 191 (IMGT 101); Km1,2 correlates with alanine 153 (IMGT A45.1) and leucine 191 (IMGT 101); and Km3 correlates with alanine 153 (IMGT A45.1) and valine 191 (IMGT V101). Optionally, one can therefore modify a sequence from one allotype to another by standard cloning approaches. For example, a L191V (IMGT L101V) change will convert a Km1,2 allotype to a Km3 allotype. For further reference on such allotypes see Jefferis and Lefranc (2009) MAbs 1(4):332-8, which is herein incorporated by reference.

Hence in one embodiment an antibody described herein contains amino acid substitutions derived from another human allotype of the same gene. In a further embodiment, the antibody contains a L191V (IMGT L101V) substitution to the kappa chain to convert the c-domain from a km1,2 to a km3 allotype.

### Antibody Binding

The antibody or fragment thereof may bind to the Vδ1 chain of a γδ TCR with a binding affinity (KD) as measured by surface plasmon resonance of less than 1.5×10⁻⁷ M (i.e. 150 nM). In a preferred embodiment, the KD is less than 1.5×10⁻⁷ M (i.e. 150 nM). In a further embodiment, the KD is 1.3×10⁻⁷ M (i.e. 130 nM) or less, such as 1.0×10⁻⁷ M (i.e. 100 nM) or less. In a yet further embodiment, the KD is less than 5.0×10⁻⁸ M (i.e. 50 nM), such as less than 4.0×10⁻⁸ M (i.e. 40 nM), less than 3.0×10⁻⁸ M (i.e. 30 nM) or less than 2.0×10⁻⁸ M (i.e. 20 nM). For example, according to one aspect, there is provided a human anti-Vδ1 antibody which binds to the Vδ1 chain of a γδ TCR with a binding affinity (KD) as measured by surface plasmon resonance of less than 1.5×10⁻⁷ M (i.e. 150 nM).

In one embodiment, the antibody or fragment thereof binds to the Vδ1 chain of a γδ TCR with a binding affinity (KD) as measured by surface plasmon resonance of less than 4.0×10⁻⁸ M (i.e. 40 nM), less than 3.0×10⁻⁸ M (i.e. 30 nM) or less than 2.0×10⁻⁸ M (i.e. 20 nM).

Binding affinity analysis of the antibodies described herein was conducted as described in Examples 1 and 5 of PCT Application No. PCT/GB2020/051956, which are herein incorporated by reference.

In one embodiment, the binding affinity of the antibody or fragment thereof is established by coating the antibody or fragment thereof directly or indirectly (e.g. by capture with an anti-human IgG Fc) onto the surface of a sensor (e.g. an amine high capacity chip or equivalent), wherein the target bound by the antibody or fragment thereof (i.e. the Vδ1 chain of a γδ TCR) is flowed over the chip to detect binding. Suitably, a MASS-2 instrument (which may also be referred to as Sierra SPR-32) is used at 25° C. in PBS+0.02% Tween 20 running buffer at 30 μl/min.

Described herein are other assays which may be used to define antibody function. For example, the antibody or fragment thereof described herein may be assessed by γδ TCR engagement, e.g. measuring downregulation of the γδ TCR upon antibody binding. Surface expression of the γδ TCR following application of the antibody or fragment thereof (optionally presented on the surface of a cell) can be measured, e.g. by flow cytometry. The antibody or fragment thereof described herein may also be assessed by measuring γδ T cell degranulation. For example, expression of CD107a, a marker for cell degranulation, can be measured following application of the antibody or fragment thereof (optionally presented on the surface of a cell) to γδ T cells, e.g. by flow cytometry. The antibody or fragment thereof described herein may also be assessed by measuring γδ T cell killing activity (to test if the antibody has an effect on the killing activity of the γδ T cell). For example, target cells may be incubated with γδ T cells in the presence of the antibody or fragment thereof (optionally presented on the surface of a cell). Following incubation, the culture may be stained with a cell viability dye to distinguish between live and dead target cells. The proportion of dead cells can then be measured, e.g. by flow cytometry.

As described herein, the antibodies or fragments thereof used in the assays may be presented on a surface, for example the surface of a cell, such as a cell comprising an Fc receptor. For example, the antibodies or fragments thereof may be presented on the surface of THP-1 cells, such as TIB-202™ cells (available from American Type Culture Collection (ATCC)). Alternatively, the antibodies or fragments thereof may be used directly in the assays.

In such functional assays, output may be measured by calculating the half maximal concentration, also referred to as “EC50” or “effective concentration at 50 percent”. The term “IC50” refers to the inhibitory concentration. Both EC50 and IC50 may be measured using methods known in the art, such as flow cytometry methods. For the avoidance of doubt, the values of EC50 in the present application are provided using IgG1 formatted antibody. Such values can be easily converted based on the molecular weight of the antibody format for equivalent values as follows:

(μg/ml)/(MW in kDa)=μM

The EC50 for downregulation of the γδ TCR upon antibody (or fragment) binding may be less than 0.50 μg/ml, such as less than 0.40 μg/ml, 0.30 μg/ml, 0.20 μg/ml, 0.15 μg/ml, 0.10 μg/ml or 0.05 μg/ml. In a preferred embodiment, the EC50 for downregulation of the γδ TCR upon antibody (or fragment) binding is less than 0.10 μg/ml. In particular, the EC50 for downregulation of the γδ TCR upon antibody (or fragment) binding may be less than 0.06 μg/ml, such as less than 0.05 μg/ml, 0.04 μg/ml or 0.03 μg/ml. In particular, said EC50 values are when the antibody is measured in an IgG1 format. For example, the EC50 γδ TCR downregulation value can be measured using flow cytometry (e.g. as described in the assay of Example 6).

The EC50 for γδ T cell degranulation upon antibody (or fragment) binding may be less than 0.050 μg/ml, such as less than 0.040 μg/ml, 0.030 μg/ml, 0.020 μg/ml, 0.015 μg/ml, 0.010 μg/ml or 0.008 μg/ml. In particular, the EC50 for γδ T cell degranulation upon antibody (or fragment) binding may be less than 0.005 μg/ml, such as less than 0.002 μg/ml. In a preferred embodiment, the EC50 for γδ T cell degranulation upon antibody (or fragment) binding is less than 0.007 μg/ml. In particular, said EC50 values are when the antibody is measured in an IgG1 format. For example, the γδ T cell degranulation EC50 value can be measured by detecting CD107a expression (i.e. a marker of cell degranulation) using flow cytometry (e.g. as described in the assay of Example 7). In one embodiment, CD107a expression is measured using an anti-CD107a antibody, such as anti-human CD107a BV421 (clone H4A3) (BD Biosciences).

The EC50 for γδ T cell killing upon the antibody (or fragment) binding may be less than 0.50 μg/ml, such as less than 0.40 μg/ml, 0.30 μg/ml, 0.20 μg/ml, 0.15 μg/ml, 0.10 μg/ml or 0.07 μg/ml. In a preferred embodiment, the EC50 for γδ T cell killing upon the antibody (or fragment) binding is less than 0.10 μg/ml. In particular, the EC50 for γδ T cell killing upon the antibody (or fragment) binding may be less than 0.060 μg/ml, such as less than 0.055 μg/ml, in particular less than 0.020 μg/ml or 0.010 μg/ml. In particular, said EC50 values are when the antibody is measured in an IgG1 format. For example, the EC50 γδ T cell killing value can be measured by detecting proportion of dead cells (i.e. using a cell viability dye) using flow cytometry following incubation of the antibody, γδ T cell and target cells (e.g. as described in the assay of Example 8). In one embodiment, death of the target cell is measured using a cell viability dye is Viability Dye eFluor™ 520 (ThermoFisher).

In the assays described in these aspects, the antibody or fragment thereof may be presented on the surface of a cell, such as a THP-1 cell, for example TIB-202™ (ATCC). The THP-1 cells are optionally labelled with a dye, such as CellTracker™ Orange CMTMR (ThermoFisher).

Functional analysis of the antibodies described herein was conducted as described in Examples 1, 6, 7 and 8 of WO2021032961 (PCT Application No. PCT/GB20201051956), which are herein incorporated by reference. A summary of binding and functional properties for the antibodies described herein are provided in Table 3.

TABLE 3
Summary of binding and functional properties for anti-Vδ1 antibodies

| | | TCR | T cell | |
|---|---|---|---|---|
| | | downregulation | degranulation | Killing assay |
| | | (EC50 μg/ml - 3 | (EC50 μg/ml - 3 | (EC50 μg/ml - 2 |
| Clone ID | K_{D} (nM) | donors) | donors) | or 3 donors) |
| 1245_P01_E07 | 12.4 | 0.04-0.11 | 0.007-0.004 | 0.06 |
| 1252_P01_C08 | 100 | 0.02-0.03 | 0.001-0.0006 | 0.02 |
| 1245_P02_G04 | 126 | 0.01-0.05 | 0.002 | 0.10 |
| 1245_P01_B07 | 341 | Positive; 0.35 | Positive; 0.1 | 0.13 |
| | | (1 donor only) | (1 donor only) | |
| 1251_P02_C05 | 1967* | Positive; N/D | Positive; N/D | N/D* |
| 1139_P01_E04 | 251 | 0.027-0.057 | 0.005 | 0.005-0.019 |
| 1245_P02_F07 | 193 | 0.032-0.043 | 0.001-0.002 | 0.006-0.018 |
| 1245_P01_G06 | 264 | 0.042-0.055 | 0.001 | 0.007-0.051 |
| 1245_P01_G09 | 208 | 0.029-0.040 | 0.001 | 0.003-0.008 |
| 1138_P01_B09 | 290 | 0.078-0.130 | N/D | 0.055-0.199 |
| 1251_P02_G10 | 829 | 0.849; N/D | N/D | N/D** |

*Binding of 1252_P02_C05 did not reach saturation, therefore data was extrapolated;
N/D: could not be determined;
N/D*: could not be determined, titration curve did not reach plateau;
N/D**: Reduced killing profile, EC50 not established.

Antibodies (or fragments) can be obtained and manipulated using the techniques disclosed for example in Green and Sambrook, Molecular Cloning: A Laboratory Manual (2012) 4th Edition Cold Spring Harbour Laboratory Press.

Monoclonal antibodies can be produced using hybridoma technology, by fusing a specific antibody-producing B cell with a myeloma (B cell cancer) cell that is selected for its ability to grow in tissue culture and for an absence of antibody chain synthesis.

A monoclonal antibody directed against a determined antigen can, for example, be obtained by:
a) immortalizing lymphocytes obtained from the peripheral blood of an animal previously immunized with a determined antigen, with an immortal cell and preferably with myeloma cells, in order to form a hybridoma,
b) culturing the immortalized cells (hybridoma) formed and recovering the cells producing the antibodies having the desired specificity.

Alternatively, the use of a hybridoma cell is not required. Antibodies capable of binding to the target antigens as described herein may be isolated from a suitable antibody library via routine practice, for example, using the phage display, yeast display, ribosomal display, or mammalian display technology known in the art. Accordingly, monoclonal antibodies can be obtained, for example, by a process comprising the steps of:
a) cloning into vectors, especially into phages and more particularly filamentous bacteriophages, DNA or cDNA sequences obtained from lymphocytes especially peripheral blood lymphocytes of an animal (suitably previously immunized with determined antigens),
b) transforming prokaryotic cells with the above vectors in conditions allowing the production of the antibodies,
c) selecting the antibodies by subjecting them to antigen-affinity selection,
d) recovering the antibodies having the desired specificity.

### Polynucleotides and Expression Vectors

Also provided are polynucleotides encoding the anti-Vδ1 antibody or fragments for use in methods of the invention. In one embodiment, the anti-Vδ1 antibody or fragment is encoded by a polynucleotide which comprises or consists of a sequence having at least 70%, such as at least 80%, such as at least 90%, such as at least 95%, such as at least 99% sequence identity with SEQ ID NO: 99-110. In one embodiment, the anti-Vδ1 antibody or fragment is encoded by an expression vector which comprises the VH region of SEQ ID NO: 99-110. In another embodiment, the anti-Vδ1 antibody or fragment is encoded by an expression vector which comprises the VL region of SEQ ID NO: 99-110. In a further embodiment the polynucleotide comprises or consists of SEQ ID NO: 99-110. In a further embodiment there is provided a cDNA comprising said polynucleotide.

In one embodiment, the polynucleotide comprises or consists of a sequence having at least 70%, such as at least 80%, such as at least 90%, such as at least 95%, such as at least 99% sequence identity with SEQ ID NO: 99-110. In one embodiment, the expression vector comprises the VH region of SEQ ID NO: 99-110. In another embodiment, the expression vector comprises the VL region of SEQ ID NO: 99-110. In a further embodiment the polynucleotide comprises or consists of SEQ ID NO: 99-110. In a further aspect there is provided a cDNA comprising said polynucleotide.

In one embodiment, the polynucleotide comprises or consists of a sequence having at least 70%, such as at least 80%, such as at least 90%, such as at least 95%, such as at least 99% sequence identity with SEQ ID NO: 99-101 or 105-108. In one embodiment, the expression vector comprises the VH region of SEQ ID NO: 99-101 or 105-108. In another embodiment, the expression vector comprises the VL region of SEQ ID NO: 99-101 or 105-108. In a further embodiment the polynucleotide comprises or consists of SEQ ID NO: 99-101 or 105-108. In a further aspect there is provided a cDNA comprising said polynucleotide.

In one embodiment, the polynucleotide comprises or consists of a sequence having at least 70%, such as at least 80%, such as at least 90%, such as at least 95%, such as at least 99% sequence identity with SEQ ID NO: 99-101. In one embodiment, the expression vector comprises the VH region of SEQ ID NO: 99-101. In another embodiment, the expression vector comprises the VL region of SEQ ID NO: 99-101. In a further embodiment the polynucleotide comprises or consists of SEQ ID NO: 99-101. In a further aspect there is provided a cDNA comprising said polynucleotide.

In one embodiment, the polynucleotide comprises or consists of a sequence having at least 70%, such as at least 80%, such as at least 90%, such as at least 95%, such as at least 99% sequence identity with any one of the portions of SEQ ID NO: 99-110 which encodes CDR1, CDR2 and/or CDR3 of the encoded immunoglobulin chain variable domain. In one embodiment, the polynucleotide comprises or consists of a sequence having at least 70%, such as at least 80%, such as at least 90%, such as at least 95%, such as at least 99% sequence identity with any one of the portions of SEQ ID NO: 99-101 or 105-108 which encodes CDR1, CDR2 and/or CDR3 of the encoded immunoglobulin chain variable domain.

In one embodiment, the polynucleotide comprises or consists of a sequence having at least 70%, such as at least 80%, such as at least 90%, such as at least 95%, such as at least 99% sequence identity with any one of the portions of SEQ ID NO: 99-101 which encodes CDR1, CDR2 and/or CDR3 of the encoded immunoglobulin chain variable domain.

In one embodiment, the polynucleotide comprises or consists of a sequence having at least 70%, such as at least 80%, such as at least 90%, such as at least 95%, such as at least 99% sequence identity with any one of the portions of SEQ ID NO: 99-110 which encodes FR1, FR2, FR3 and/or FR4 of the encoded immunoglobulin chain variable domain. In one embodiment, the polynucleotide comprises or consists of a sequence having at least 70%, such as at least 80%, such as at least 90%, such as at least 95%, such as at least 99% sequence identity with any one of the portions of SEQ ID NO: 99-101 or 105-108 which encodes FR1, FR2, FR3 and/or FR4 of the encoded immunoglobulin chain variable domain.

In one embodiment, the polynucleotide comprises or consists of a sequence having at least 70%, such as at least 80%, such as at least 90%, such as at least 95%, such as at least 99% sequence identity with any one of the portions of SEQ ID NO: 99-101 which encodes FR1, FR2, FR3 and/or FR4 of the encoded immunoglobulin chain variable domain.

The polynucleotides and expression vectors of the invention may also be described in reference to the amino acid sequence encoded. Therefore, in one embodiment, the polynucleotide comprises or consists of a sequence encoding the amino acid sequence of any one of SEQ ID NOs: 62 to 85. In one embodiment, the expression vector comprises a sequence encoding the amino acid sequence of any one of SEQ ID NOs: 62 to 73. In another embodiment, the expression vector comprises a sequence encoding the amino acid sequence of any one of SEQ ID NOs: 74 to 85.

To express the antibodies, or fragments thereof, polynucleotides encoding partial or full-length light and heavy chains, as described herein, are inserted into expression vectors such that the genes are operatively linked to transcriptional and translational control sequences. Therefore, in one embodiment, the expression vector comprises the VH region of SEQ ID NO: 99-110, such as SEQ ID NO: 99, 100, 101, 105, 106, 107 or 108. In another embodiment, the expression vector comprises the VL region of SEQ ID NO: 99-110, such as SEQ ID NO: 99, 100, 101, 105, 106, 107 or 108.

It will be understood that the nucleotide sequences described herein comprise additional sequences encoding amino acid residues to aid with translation, purification and detection, however alternative sequences may be used depending upon the expression system used. For example, the initial (5′-end) nine nucleotides of SEQ ID NOs: 99-110 and the final (3′-end) 36 nucleotides of SEQ ID NOs: 99-100, 102-103, 105-110, or the final (3′-end) 39 nucleotides of SEQ ID NOs: 101 and 104 are optional sequences. These optional sequences can be removed, modified or substituted if alternate design, translation, purification or detection strategies are adopted.

Mutations can be made to the DNA or cDNA that encode polypeptides which are silent as to the amino acid sequence of the polypeptide, but which provide preferred codons for translation in a particular host. The preferred codons for translation of a nucleic acid in, e.g., E. coli and S. cerevisiae, as well as mammalian, specifically human, are known.

Mutation of polypeptides can be achieved for example by substitutions, additions or deletions to a nucleic acid encoding the polypeptide. The substitutions, additions or deletions to a nucleic acid encoding the polypeptide can be introduced by many methods, including for example error-prone PCR, shuffling, oligonucleotide-directed mutagenesis, assembly PCR, PCR mutagenesis, in vivo mutagenesis, cassette mutagenesis, recursive ensemble mutagenesis, exponential ensemble mutagenesis, site-specific mutagenesis, gene reassembly, artificial gene synthesis, Gene Site Saturation Mutagenesis (GSSM), synthetic ligation reassembly (SLR) or a combination of these methods. The modifications, additions or deletions to a nucleic acid can also be introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, ensemble mutagenesis, chimeric nucleic acid multimer creation, or a combination thereof.

In particular, artificial gene synthesis may be used. A gene encoding a polypeptide described herein can be synthetically produced by, for example, solid-phase DNA synthesis. Entire genes may be synthesized de novo, without the need for precursor template DNA. To obtain the desired oligonucleotide, the building blocks are sequentially coupled to the growing oligonucleotide chain in the order required by the sequence of the product Upon the completion of the chain assembly, the product is released from the solid phase to solution, deprotected, and collected. Products can be isolated by high-performance liquid chromatography (HPLC) to obtain the desired oligonucleotides in high purity.

Expression vectors include, for example, plasmids, retroviruses, cosmids, yeast artificial chromosomes (YACs) and Epstein-Barr virus (EBV) derived episomes. The polynucleotide is ligated into a vector such that transcriptional and translational control sequences within the vector serve their intended function of regulating the transcription and translation of the polynucleotide. Expression and/or control sequences can include promoters, enhancers, transcription terminators, a start codon (i.e. ATG) 5′ to the coding sequence, splicing signals for introns and stop codons. The expression vector and expression control sequences are chosen to be compatible with the expression host cell used. SEQ ID NOs: 99-110 comprise the nucleotide sequences encoding single chain variable fragments of the invention, comprising a VH region and a VL region joined by a synthetic linker (e.g. encoding SEQ ID NO: 98). It will be understood that polynucleotides or expression vectors of the invention may comprise the VH region, the VL region or both (optionally including the linker). Therefore, polynucleotides encoding the VH and VL regions can be inserted into separate vectors, alternatively sequences encoding both regions are inserted into the same expression vector. The polynucleotide(s) are inserted into the expression vector by standard methods (e.g., ligation of complementary restriction sites on the polynucleotide and vector, or blunt end ligation if no restriction sites are present).

A convenient vector is one that encodes a functionally complete human CH or CL immunoglobulin sequence, with appropriate restriction sites engineered so that any VH or VL sequence can be easily inserted and expressed, as described herein. The expression vector can also encode a signal peptide that facilitates secretion of the antibody (or fragment thereof) from a host cell. The polynucleotide may be cloned into the vector such that the signal peptide is linked in-frame to the amino terminus of the antibody. The signal peptide can be an immunoglobulin signal peptide or a heterologous signal peptide (i.e., a signal peptide from a non-immunoglobulin protein).

A host cell may comprise a first vector encoding the light chain of the antibody or fragment thereof, and a second vector encoding the heavy chain of the antibody or fragment thereof. Alternatively, the heavy and light chains both encoded on the same expression vector introduced into the host cell. In one embodiment, the polynucleotide or expression vector encodes a membrane anchor or transmembrane domain fused to the antibody or fragment thereof, wherein the antibody or fragment thereof is presented on an extracellular surface of the host cell.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors.

Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. Antigen-binding fragments of antibodies such as the scFv and Fv fragments can be isolated and expressed in E. coli using methods known in the art.

The antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Antibodies (or fragments) of the invention can be obtained and manipulated using the techniques disclosed for example in Green and Sambrook, Molecular Cloning: A Laboratory Manual (2012) 4th Edition Cold Spring Harbour Laboratory Press.

Monoclonal antibodies can be produced using hybridoma technology, by fusing a specific antibody-producing B cell with a myeloma (B cell cancer) cell that is selected for its ability to grow in tissue culture and for an absence of antibody chain synthesis.

A monoclonal antibody directed against a determined antigen can, for example, be obtained by:
a) immortalizing lymphocytes obtained from the peripheral blood of an animal previously immunized with a determined antigen, with an immortal cell and preferably with myeloma cells, in order to form a hybridoma,
b) culturing the immortalized cells (hybridoma) formed and recovering the cells producing the antibodies having the desired specificity.

Alternatively, the use of a hybridoma cell is not required. Antibodies capable of binding to the target antigens as described herein may be isolated from a suitable antibody library via routine practice, for example, using the phage display, yeast display, ribosomal display, or mammalian display technology known in the art. Accordingly, monoclonal antibodies can be obtained, for example, by a process comprising the steps of:
a) cloning into vectors, especially into phages and more particularly filamentous bacteriophages, DNA or cDNA sequences obtained from lymphocytes especially peripheral blood lymphocytes of an animal (suitably previously immunized with determined antigens),
b) transforming prokaryotic cells with the above vectors in conditions allowing the production of the antibodies,
c) selecting the antibodies by subjecting them to antigen-affinity selection,
d) recovering the antibodies having the desired specificity.

### Treatment Methods

The compositions of the invention find particular use in clinical applications. On top of the anti-cancer and healthy cell sparing activity of the trinity of immune cell types present in the composition, NK cells are also very potent to fight of infectious disease, caused by viruses, bacteria or fungi. Vδ1 T cells have an established track record of being fundamental for the recognition and protection against CMV reactivation, a significant problem in immune compromised patients as in the case of transplant patients or pre-treated cancer patients. CMV activated Vδ1 actually significantly reduce the risk of secondary malignancies in patients on broad immune suppressants after transplantation.

Vδ2 T cells are unique in that they recognise with very high affinity intermediates of the non-mevalonate pathway, namely HMBPP, which is exclusively used by prokaryotes and some eukaryotes. These cells are fundamentally important in recognising and fighting mycobacterial infections and can represent more than 50% of all T cells in the blood of patients with active infections. Vδ2 also recognise protozoa and contribute greatly to the immune response against Malaria.

Hence, the composition described herein would provide broad protection against cancer therapy or transplantation induced complications such as viral reactivation and bacterial infections which can contribute to mortalities in immunotherapies. A protection that one cell type at a time would not be able to cover.

For the same reasons, the composition is also attractive for the treatment of infectious diseases. In fact NK cells have been trialed for the treatment of coronavirus (COVID-19). Therefore, the composition may be used to treat COVID, CMV, HIV, malaria and any mycobacterial infection.

According to a further aspect of the invention, there is provided a composition as defined herein for use in therapy.

According to a further aspect of the invention, there is provided a composition as defined herein for use in a method of treating cancer, an infectious disease or an inflammatory disease.

The cell populations and compositions obtained by methods of the invention may also be used in therapy. Therefore, according to a further aspect of the invention, there is provided the cell population obtained by a method as defined herein for use as a medicament.

References herein to a cell population “for use” as a medicament or in therapy are limited to administration of the cell population to a subject.

In one embodiment, the cell population is for use in the treatment of cancer, an infectious disease or an inflammatory disease. In a further embodiment, the cell population is for use in the treatment of cancer.

According to a further aspect of the invention, there is provided the pharmaceutical composition comprising the cell population as defined herein for use as a medicament. In one embodiment, the pharmaceutical composition comprising the cell population is for use in the treatment of cancer, an infectious disease or an inflammatory disease. In a further embodiment, the pharmaceutical composition comprising the cell population is for use in the treatment of cancer.

According to a further aspect of the invention, there is provided a method of modulating an immune response in a subject in need thereof comprising administering a therapeutically effective amount of the composition as defined herein.

According to a further aspect of the invention, there is provided a method of treating a cancer, an infectious disease or an inflammatory disease in a subject in need thereof, comprising administering a therapeutically effective amount of a composition comprising NK cells and γδT cells wherein at least 40% of the γδ T cells present in the composition express CD56. It will be understood that embodiments described for the compositions hereinbefore, may equally be applied to this aspect of the invention.

According to a further aspect of the invention, there is provided a method of treating a cancer, an infectious disease or an inflammatory disease in a subject in need thereof, comprising administering a therapeutically effective amount of a composition comprising cells wherein at least 90% of the cells consist of NK cells and γδ T cells, wherein at least 10% of the cells are Vδ1 T cells, at least 5% of the cells are Vδ2 T cells and at least 30% of the cells are NK cells.

According to further aspects of the invention, there is provided the use of the cell population as defined herein for the manufacture of a medicament, for example in the treatment of cancer, an infectious disease or an inflammatory disease.

### Adoptive T Cell Therapy

Compositions obtained by the methods of the invention may be used as a medicament, for example for adoptive T cell therapy. This involves the transfer of immune cells into a patient. The therapy may be autologous, i.e. the immune cells may be transferred back into the same patient from which they were obtained, or the therapy may be allogeneic, i.e. the immune cells from one person may be transferred into a different patient. In instances involving allogeneic transfer, the composition may be substantially free of αβ T cells. For example, as T cells may be depleted from the composition, e.g., after expansion, using any suitable means known in the art (e.g., by negative selection, e.g., using magnetic beads). A method of treatment may include: providing a sample obtained from a donor individual; culturing immune cells obtained from the sample as described herein, e.g. to produce an expanded population; and administering the cultured immune cells to a recipient individual.

The patient or subject to be treated is preferably a human cancer patient (e.g., a human cancer patient being treated for a solid tumour) or a virus-infected patient (e.g., a CMV-infected or HIV infected patient). In some instances, the patient has and/or is being treated for a solid tumour. Because they are normally resident in non-haematopoietic tissues, tissue-resident Vδ1 T cells are also more likely to home to and be retained within tumour masses than their systemic blood-resident counterparts and adoptive transfer of these cells is likely to be more effective at targeting solid tumours and potentially other non-haematopoietic tissue-associated immunopathologies.

As γδ T cells and NK cells are non-MHC restricted, they do not recognize a host into which they are transferred as foreign, which means that they are less likely to cause graft-versus-host disease. This means that they can be used “off the shelf” and transferred into any recipient, e.g., for allogeneic adoptive T cell therapy.

MHC unrestricted lymphocytes obtained by methods described herein may express NKG2D and respond to a NKG2D ligand (e.g. MICA), which is strongly associated with malignancy. They may also express a cytotoxic profile in the absence of any activation and are therefore likely to be effective at killing tumour cells.

In some embodiments, a method of treatment of an individual with a tumour may include; providing a sample of said tumour obtained from a donor individual, culturing the MHC unrestricted lymphocytes obtained from the sample as described above, and; administering the population of MHC unrestricted lymphocytes to the individual with the tumour.

In some instances, a therapeutically effective amount of MHC unrestricted lymphocytes obtained by the any of the methods described above can be administered in a therapeutically effective amount to a subject (e.g., for treatment of cancer, e.g. for treatment of a solid tumour). In some cases, the therapeutically effective amount of MHC unrestricted lymphocytes is less than 10×10¹² cells per dose (e.g., less than 9×10¹² cells per dose, less than 8×10¹² cells per dose, less than 7×10¹² cells per dose, less than 6×10¹² cells per dose, less than 5×10¹² cells per dose, less than 4×10¹² cells per dose, less than 3×10¹² cells per dose, less than 2×10¹² cells per dose, less than 1×10¹² cells per dose, less than 9×10¹¹ cells per dose, less than 8×10¹¹ cells per dose, less than 7×10¹¹ cells per dose, less than 6×10¹¹ cells per dose, less than 5×10¹¹ cells per dose, less than 4×10¹¹ cells per dose, less than 3×10¹ cells per dose, less than 2×10¹¹ cells per dose, less than 1×10¹¹ cells per dose, less than 9×10¹⁰ cells per dose, less than 7.5×10¹⁰ cells per dose, less than 5×10¹⁰ cells per dose, less than 2.5×10¹⁰ cells per dose, less than 1×10¹⁰ cells per dose, less than 7.5×10⁹ cells per dose, less than 5×10⁹ cells per dose, less than 2.5×10⁹ cells per dose, less than 1×10⁹ cells per dose, less than 7.5×10⁸ cells per dose, less than 5×10⁸ cells per dose, less than 2.5×10⁸ cells per dose, less than 1×10⁸ cells per dose, less than 7.5×10⁷ cells per dose, less than 5×10⁷ cells per dose, less than 2.5×10⁷ cells per dose, less than 1×10⁷ cells per dose, less than 7.5×10⁸ cells per dose, less than 5×10⁶ cells per dose, less than 2.5×10⁶ cells per dose, less than 1×10⁶ cells per dose, less than 7.5×10⁵ cells per dose, less than 5×10⁵ cells per dose, less than 2.5×10⁵ cells per dose, or less than 1×10⁵ cells per dose).

In some embodiments, the therapeutically effective amount of MHC unrestricted lymphocytes is less than 10×10¹² cells over the course of treatment (e.g., less than 9×10¹² cells, less than 8×10¹² cells, less than 7×10¹² cells, less than 6×10¹² cells, less than 5×10¹² cells, less than 4×10¹² cells, less than 3×10¹² cells, less than 2×10¹² cells, less than 1×10¹² cells, less than 9×10¹² cells, less than 8×10¹¹ cells, less than 7×10¹¹ cells, less than 6×10¹¹ cells, less than 5×10¹¹ cells, less than 4×10¹¹ cells, less than 3×10¹¹ cells, less than 2×10¹¹ cells, less than 1×10¹¹ cells, less than 9×10¹⁰ cells, less than 7.5×10¹⁰ cells, less than 5×10¹⁰ cells, less than 2.5×10¹⁰ cells, less than 1×10¹⁰ cells, less than 7.5×10¹⁰ cells, less than 5×10⁹ cells, less than 2.5×10⁹ cells, less than 1×10⁹ cells, less than 7.5×10⁸ cells, less than 5×10⁸ cells, less than 2.5×10⁸ cells, less than 1×10⁸ cells, less than 7.5×10⁷ cells, less than 5×10⁷ cells, less than 2.5×10⁷ cells, less than 1×10⁷ cells, less than 7.5×10⁶ cells, less than 5×10⁶ cells, less than 2.5×10⁶ cells, less than 1×10⁶ cells, less than 7.5×10⁵ cells, less than 5×10⁵ cells, less than 2.5×10⁵ cells, or less than 1×10⁵ cells over the course of treatment).

In some embodiments, a dose of MHC unrestricted lymphocytes as described herein comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of MHC unrestricted lymphocytes comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of MHC unrestricted lymphocytes comprises about 1.1×10⁶-1.8×10⁷ cells/kg. In some embodiments, a dose of MHC unrestricted lymphocytes comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of MHC unrestricted lymphocytes comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of MHC unrestricted lymphocytes comprises up to about 1×10⁷, 2×10⁷, 5×10⁷, 1×10⁸, 2×10⁸, 5×10⁸, 1×10⁸, 2×10⁹, or 5×10⁹ cells.

In one embodiment, the subject is administered 104 to 10⁶ MHC unrestricted lymphocytes per kg body weight of the subject In one embodiment, the subject receives an initial administration of a population of MHC unrestricted lymphocytes (e.g., an initial administration of 104 to 108 MHC unrestricted lymphocytes per kg body weight of the subject, e.g., 104 to 108 MHC unrestricted lymphocytes per kg body weight of the subject), and one or more (e.g., 2, 3, 4, or 5) subsequent administrations of MHC unrestricted lymphocytes (e.g., one or more subsequent administration of 104 to 108 MHC unrestricted lymphocytes per kg body weight of the subject, e.g., 104 to 108 MHC unrestricted lymphocytes per kg body weight of the subject). In one embodiment, the one or more subsequent administrations are administered less than 15 days, e.g., 14, 13, 12, 11, 10, 9, 8, 7, 6, 5, 4, 3, or 2 days after the previous administration, e.g., less than 4, 3, or 2 days after the previous administration. In one embodiment, the subject receives a total of about 108 MHC unrestricted lymphocytes per kg body weight of the subject over the course of at least three administrations of a population of MHC unrestricted lymphocytes, e.g., the subject receives an initial dose of 1×10⁵ MHC unrestricted lymphocytes, a second administration of 3×10⁵ MHC unrestricted lymphocytes, and a third administration of 6×10⁵ MHC unrestricted lymphocytes, and, e.g., each administration is administered less than 4, 3, or 2 days after the previous administration.

In some embodiments, one or more additional therapeutic agents can be administered to the subject. The additional therapeutic agent may be selected from the group consisting of an immunotherapeutic agent, a cytotoxic agent, a growth inhibitory agent, a radiation therapy agent, an anti-angiogenic agent, or a combination of two or more agents thereof. The additional therapeutic agent may be administered concurrently with, prior to, or after administration of the MHC unrestricted lymphocytes. The additional therapeutic agent may be an immunotherapeutic agent, which may act on a target within the subject's body (e.g., the subject's own immune system) and/or on the transferred MHC unrestricted lymphocytes.

The administration of the compositions may be carried out in any convenient manner. The compositions described herein may be administered to a patient transarterially, subcutaneously, intradermally, intratumorally, intranodally, intramedullary, intramuscularly, by intravenous injection, or intraperitoneally, e.g., by intradermal or subcutaneous injection. The compositions of MHC unrestricted lymphocytes may be injected directly into a tumour, lymph node, or site of infection.

### Gene Engineering

The MHC unrestricted lymphocytes cells obtained by the method of the invention may also be gene engineered for enhanced therapeutic properties, such as for Chimeric Antigen Receptor T cell (CAR-T) therapy. This involves the generation of engineered receptors (e.g. CARs) to re-program the T cell with a new specificity, e.g. the specificity of a monoclonal antibody. The engineered receptor may make the lymphocytes specific for malignant cells and therefore useful for cancer immunotherapy. For example, the lymphocytes may recognize cancer cells expressing a tumour antigen, such as a tumour associated antigen that is not expressed by normal somatic cells from the subject tissue. Thus, the CAR-modified lymphocytes may be used for adoptive T cell therapy of, for example, cancer patients.

It will be understood that all embodiments described herein may be applied to all aspects of the invention.

### CLAUSES

A set of clauses defining the invention and its preferred aspects is as follows:

1. An isolated composition comprising Natural Killer (NK) cells and γδ T cells wherein at least 40% of the γδ T cells present in the composition are CD56^{bright}.

2. An isolated composition comprising NK cells and γδ T cells wherein at least 50% of the NK cells present in the composition are CD56^{bright}.

3. An isolated composition comprising NK cells and γδ T cells wherein at least 50% of the γδ T cells present in the composition express CD56.

4. The isolated composition of any one of clauses 1 to 3, wherein at least 80% of the cells present in the composition comprise NK cells and γδ T cells.

5. The isolated composition of any one of clauses 1 to 4, wherein at least 90% of the cells present in the composition consist of NK cells and γδ T cells.

6. The isolated composition of any one of clauses 1 to 5, wherein at least 20% of the cells present in the composition are γδ T cells.

7. The isolated composition of any one of clauses 1 to 6, wherein at least 30% of the cells present in the composition are γδ T cells.

8. The isolated composition of any one of clauses 1 to 7, wherein less than 80% of the cells present in the composition are γδ T cells.

9. The isolated composition of any one of clauses 1 to 8, wherein the γδ T cells comprise Vδ1 T cells and Vδ2 T cells.

10. The isolated composition of any one of clauses 1 to 9, wherein at least 20% of the cells present in the composition are NK cells.

11. The isolated composition of any one of clauses 1 to 10, wherein at least 30% of the cells present in the composition are NK cells.

12. An isolated composition comprising cells wherein at least 90% of the cells consist of NK cells and γδ T cells, wherein at least 10% of the cells are Vδ1 T cells, at least 5% of the cells are Vδ2 T cells and at least 30% of the cells are NK cells.

13. The isolated composition of clause 12, wherein at least 40% of the γδ T cells present in the composition express CD56.

14. The isolated composition of clause 12 or clause 13, wherein less than 80% of the cells present in the composition are γδ T cells.

15. The isolated composition of any one of clauses 1 to 14, wherein the γδ T cells comprise Vδ1 T cells and at least 15% of said Vδ1 T cells express NKp30.

16. The isolated composition of any one of clauses 1 to 15, wherein the γδ T cells comprise Vδ1 T cells and less than 50% of said Vδ1 T cells express CD27.

17. The isolated composition of any one of clauses 1 to 16, wherein the isolated composition comprises engineered NK cells and γδ T cells.

18. The isolated composition of clause 17, wherein the engineered NK cells and γδ T cells express a chimeric antigen receptor.

19. The isolated composition of any one of clauses 1 to 18, for use in therapy.

20. The isolated composition of any one of clauses 1 to 18, for use in a method of treating cancer, an infectious disease or an inflammatory disease.

21. A method of expanding non-γδ+ MHC unrestricted lymphocytes comprising stimulating a mixed cell population comprising γδ T cells and NK cells using an anti-TCR delta variable 1 (anti-Vδ1) antibody or fragment thereof, in the presence of Interleukin-15 (IL-15) and in the absence of Interleukin-4 (IL-4) and culturing the mixed cell population.

22. The method of clause 21, which comprises culturing the mixed cell population for at least 7 days, such as at least 14 days.

23. The method of any one of clauses 21 or 22, wherein at least 50% of the expanded non-γδ+ MHC unrestricted lymphocytes are CD56^{bright}.

24. A method of preparing a composition comprising a cell population enriched for MHC unrestricted lymphocytes, wherein the method comprises:
(1) culturing a sample obtained from a subject in the presence of:
   (i) an anti-Vδ1 antibody or fragment thereof; and
   (ii) IL-15, in the absence of IL-4, from the first day of said culturing; and
(2) isolating the cell population cultured from the sample.

25. The method of clause 24, wherein step (1) of the method comprises culturing the sample for at least 7 days, such as at least 14 days.

26. The method of clause 24 or clause 25, wherein the MHC unrestricted lymphocytes comprise NK cells and γδ T cells.

27. The method of any one of clauses 24 to 26, wherein at least 70% of the MHC unrestricted lymphocytes present in the cell population isolated in step (2) express CD56.

28. The method of any one of clauses 24 to 27, wherein the cell population isolated in step (2) comprises γδ T cells and at least 40% of the γδ T cells express CD56.

29. The method of any one of clauses 24 to 28, wherein less than 80% of the cell population isolated in step (2) are γδ T cells.

30. The method of any one of clauses 24 to 29, wherein at least 90% of the cell population isolated in step (2) consists of NK cells and γδ T cells and wherein at least 10% of the cells are Vδ1 T cells, at least 5% of the cells are Vδ2 T cells and at least 30% of the cells are NK cells.

31. The method of any one of clauses 21 to 30, wherein the activating anti-Vδ1 antibody or fragment thereof, binds to an epitope of a variable delta 1 (Vδ1) chain of a γδ T cell receptor (TCR) comprising one or more amino acid residues within amino acid regions:
(i) 3-20 of SEQ ID NO: 1; and/or
(ii) 37-77 of SEQ ID NO: 1.

32. The method of clause 31, wherein the epitope comprises one or more amino acid residues within amino acid regions: 5-20 and 62-77; 50-84; 37-53 and 59-72; 59-77; or 3-17 and 62-89, of SEQ ID NO: 1.

33. The method of any one of clauses 21 to 32, wherein the anti-Vδ1 antibody or fragment thereof comprises one or more of
a CDR3 comprising a sequence having at least 80% sequence identity with any one of SEQ ID NOs: 2-25;
a CDR2 comprising a sequence having at least 80% sequence identity with any one of SEQ ID NOs: 26-37 and SEQUENCES: A1-A12 (of Table 2); and/or
a CDR1 comprising a sequence having at least 80% sequence identity with any one of SEQ ID NOs: 38-81.

34. The method of any one of clauses 21 to 33, wherein the anti-Vδ1 antibody or fragment thereof comprises a VH region comprising an amino acid sequence having at least 80% sequence identity with any one of SEQ ID NOs: 62-73.

35. The method of any one of clauses 21 to 34, wherein the anti-Vδ1 antibody or fragment thereof comprises a VL region comprising an amino acid sequence having at least 80% sequence identity with any one of SEQ ID NOs: 74-85.

36. The method of any one of clauses 21 to 35, wherein the anti-Vδ1 antibody or fragment thereof comprises an amino acid sequence of any one of SEQ ID NOs: 86-97.

37. The method of any one of clauses 24 to 36, wherein the sample is a haematopoietic sample or a fraction thereof.

38. The method of clause 37, wherein the haematopoietic sample is selected from peripheral blood, umbilical cord blood, lymphoid tissue, thymus, bone marrow, lymph node tissue or fractions thereof.

39. The method of clause 37 or clause 38, wherein the haematopoietic sample consists of low density mononuclear cells (LDMCs) or peripheral blood mononuclear cells (PBMCs).

40. The method of any one of clauses 24 to 39, wherein the sample is obtained from human or non-human animal tissue.

41. The method of any one of clauses 24 to 40, wherein the sample is enriched for T cells prior to said culturing.

42. The method of any one of clauses 24 to 41, wherein the sample is depleted of αβ T cells prior to said culturing.

43. The method of any one of clauses 24 to 42, wherein the culturing is performed in media comprising 2.5% plasma.

44. A composition obtained by the method of any one of clauses 21 to 43.

45. The composition of clause 44 for use in therapy.

48. The composition of clause 44 for use in a method of treating cancer, an infectious disease or an inflammatory disease.

47. A method of treating a cancer, an infectious disease or an inflammatory disease in a subject in need thereof, comprising administering a therapeutically effective amount of a composition comprising NK cells and γδ T cells wherein at least 40% of the γδ T cells present in the composition express CD56.

48. A method of treating a cancer, an infectious disease or an inflammatory disease in a subject in need thereof, comprising administering a therapeutically effective amount of a composition comprising cells wherein at least 90% of the cells consist of NK cells and γδ T cells, wherein at least 10% of the cells are Vδ1 T cells, at least 5% of the cells are Vδ2 T cells and at least 30% of the cells are NK cells.

49. The method of any one of clauses 24 to 43, wherein step (1) of the method comprises culturing the sample for at least 12 days, such as for about 12 days.

50. A cell population enriched for MHC unrestricted lymphocytes obtained by the method of any one of clauses 21 to 43 or clause 49.

51. The cell population of clause 50 for use in therapy.

52. The cell population of clause 50 for use in a method of treating cancer, an infectious disease or an inflammatory disease.

53. A composition comprising a cell population obtainable by the method of any one of clauses 21 to 43.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art. The invention will now be described using the following, non-limiting examples:

### EXAMPLES

### Example 1. Materials and Methods

### Cell Expansions

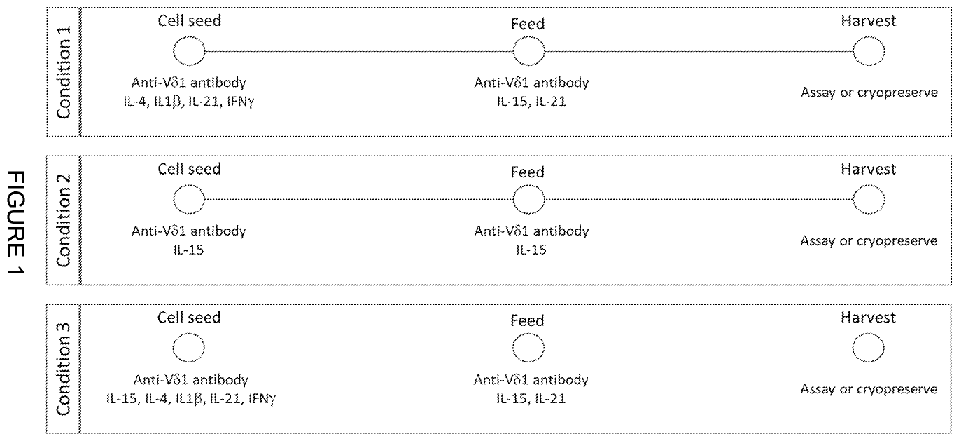

Cells were expanded under conditions described below and as summarised in FIG. 1.

Condition 1: Vδ1+γδ T cells were generated from αβ-TCR+ depleted peripheral blood mononuclear cells using serum-free culture medium (CTS OpTmizer, Thermo Fisher) supplemented with 2.5% pooled allogeneic plasma (Octaplas, Octapharma) and Glutamax (ThermoFisher). The isolated cells were seeded in the presence of recombinant IL-4, IL-1β, IL-21 and IFNγ and soluble anti-Vδ1 monoclonal antibody (1252_P01_C08—in house antibody clone). After setup, cultures were incubated at 37° C. and 5% CO₂ in a humidified incubator. Expanding cells were re-supplemented with fresh IL-15, IL-21 and anti-Vδ1 antibody. Cells were harvested after 11-14 days of culture and cryopreserved as a mixed population in Cryostor5 (STEMCELL Technologies).

Condition 2: CD56+ lymphocytes were generated from αβ-TCR+ depleted peripheral blood mononuclear cells using serum-free culture medium (CTS OpTmizer, Thermo Fisher) supplemented with 2.5% pooled allogeneic plasma (Octaplas, Octapharma) and Glutamax (ThermoFisher). The isolated cells were grown in the presence of recombinant IL-15 and soluble anti-Vδ1 monoclonal antibody (in house antibody clone). After setup, cultures were incubated at 37° C. and 5% CO₂ in a humidified incubator. Expanding cells were regularly re-supplemented with fresh IL-15 and anti-Vδ1 antibody. Cells were harvested after 11-14 days of culture and cryopreserved as a mixed population in Cryostor5 (STEMCELL Technologies).

Condition 3: Vδ1+γδ T cells were generated from αβ-TCR+ depleted peripheral blood mononuclear cells as described for condition 1. However, in addition to recombinant IL-4, IL-1β, IL-21 and IFNγ and soluble anti-Vδ1 monoclonal antibody (in house antibody clone), cells were also seeded in the presence recombinant IL-15. After setup, cultures were incubated at 37° C. and 5% CO₂ in a humidified incubator. Expanding Cells were re-supplemented with fresh IL-15, IL-21 and anti-Vδ1 antibody. Cells were harvested after 11-14 days of culture and cryopreserved as a mixed population in Cryostor5 (STEMCELL Technologies).

### Flow Cytometry Assessment of Cell Purity and Surface Phenotype

Immunophenotyping was performed using a MACSQuant16 flow cytometer. Dead cells were excluded using eFluor 780 fixable viability dye. Cells were analysed for the expression of surface markers using FITC anti-CD45, PE anti-TCR α/β, APC anti-TCR γ/δ, VioBlue anti-TCR Vδ1, FITC anti-CD27, PE anti-NKp30, PE-Vio770 anti-NKG2D, PE anti-CD19, PerCP-Vio770 anti-CD14, PE-Vio770 anti-CD56, APC anti-CD3, PE anti-TCR Vδ3 and PE-Vio770 anti-TCR Vδ2 antibodies, available from Miltenyi and BioLegend.

Transduction with Lentivirus Encoding CD19 Chimeric Antigen Receptors

Expanding cells from conditions 1 and 2 were transduced with lentiviral vectors encoding CD19-targeting chimeric antigen receptors in RetroNectin coated (20 μg/mL) non-tissue culture treated 24-well plates. Viral vector was mixed with immune cells diluted in CTS OpTmizer (supplemented with IL-15 and anti-Vδ1 antibody) and spinoculated at 800×g for 45 minutes at 32° C. Transduction efficiency was determined by flow cytometry four days post-transduction.

### Thawing Cryopreserved Cultures

Frozen cryovials were thawed in a 37° C. water bath and added to pre-warmed OpTmizer+2.5% allogeneic plasma. Cells were spun down at 300 g for 7 minutes, counted and viability assessed, and then resuspended at 2×10⁶ cells per mL for phenotyping and downstream assays.

### Cytotoxicity Assays

Expanded cells from conditions 1 to 3 were co-cultured with CellTrace Violet-labelled NALM-6 tumour target cells in a 96-well round bottom plate, at various effector to target ratios. The control (Ctrl) tested target cells alone, without the presence of effector cells. After 20 hours, SYTOX AADVANCED Dead Cell Stain was added to the cultures before acquisition on a MACSQuant10 flow cytometer. CellTrace Violet (CTV) staining identified NALM-6 tumour cells and positivity for SYTOX discriminated dead cells from viable cells.

### Example 2. Comparison of Cell Culture Conditions

Cell populations were expanded using one of conditions 1-3 described in Example 1 and as summarised in FIG. 1. The expanded cell populations were then analysed and compared.

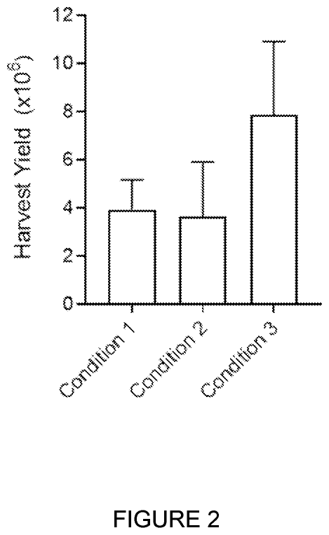

Cell yield at harvest is shown in FIG. 2. Condition 2 produces equivalent cell yields to condition 1. Condition 3 increases cell yield relative to condition 1.

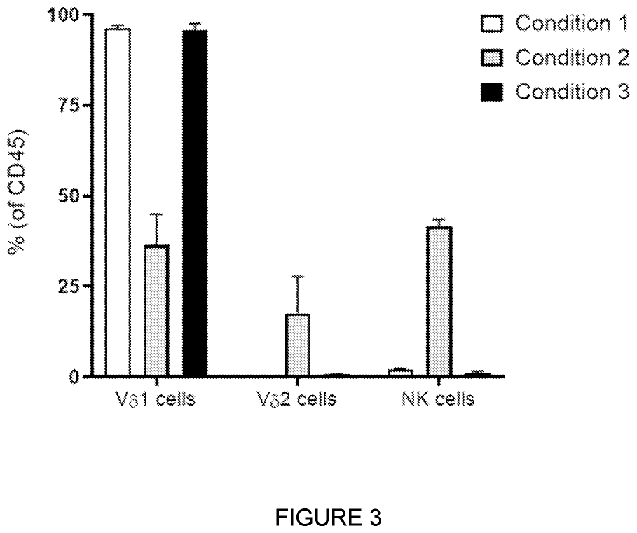

The immune cell composition was analysed. Conditions 1 and 3 produce a population highly enriched for Vδ1 T cells, however condition 2 produced a mixed population comprising of Vδ1+, Vδ2+ and NK cells. Therefore, condition 2 unexpectedly showed simultaneous enrichment for all MHC unrestricted lymphocytes. Results are shown in FIG. 3. Individual cellular composition from three donors cultured using condition 2 is summarised in Table 4.

TABLE 4
Cell composition obtained from 3 donors under condition 2

| | | | | | | Vδ1 − Vδ2 − |
|---|---|---|---|---|---|---|
| | NK | γδ T | Vδ1 T | Vδ2 T | Vδ3 T | Vδ3 − γδ T |
| | cells | cells | cells | cells | cells | cells |
| Donor # | (%) | (%) | (%) | (%) | (%) | (%) |
| LK012R2-01 | 38.3 | 54.5 | 19.1 | 34.7 | 0.3 | 0.1 |
| LK033R1-01 | 64.1 | 31.02 | 13.07 | 15.34 | 2.2 | 0.23 |
| LK021R2-01 | 36 | 58.8 | 51.27 | 7.06 | 0.13 | 0.27 |

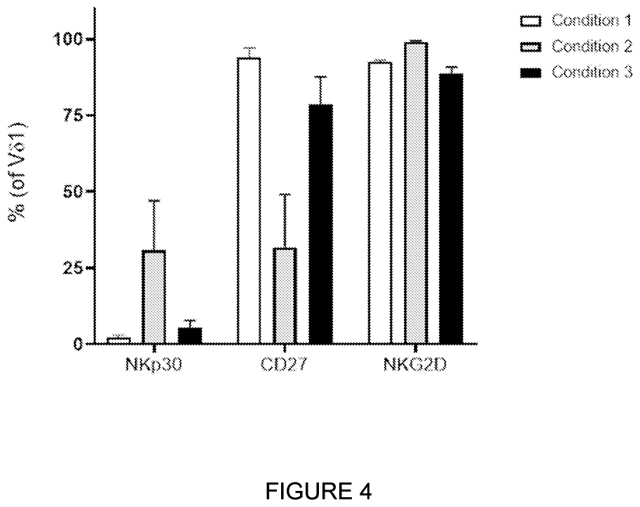

The surface phenotype of Vδ1 T cells was analysed. Vδ1 T cells derived from conditions 1 and 3 are positive for CD27 and NKG2D. Vδ1 T cells derived from condition 2 express less CD27 but more NKp30 than conditions 1 and 3 which is consistent with an effector phenotype. Results are shown in FIG. 4.

### Example 3. Comparison of Anti-Vδ1 Antibodies

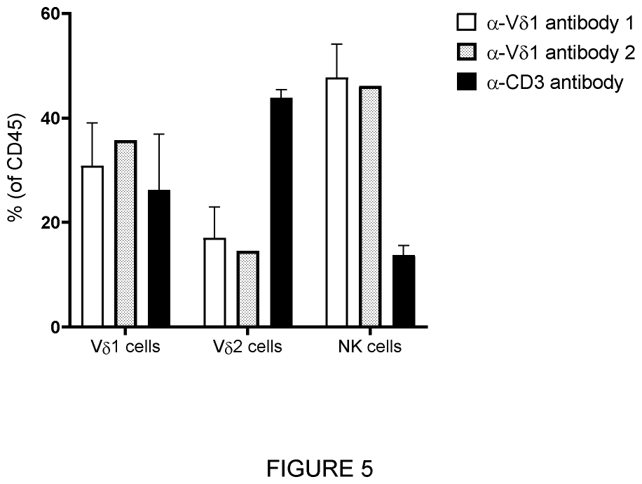

Cell populations were expanded using condition 2 but in the presence of different antibodies. Results are shown in FIG. 5. The use of two separate in-house anti-Vδ1 antibody clones (1252_P01_C08 and 1245_P01_B07) in condition 2 enriches for NK cells as well as γδ cells. This is in comparison to the anti-CD3 antibody (OKT-3, BioLegend) which only enriches effectively for γδ T cells.

### Example 4. Cell Composition Cytotoxicity

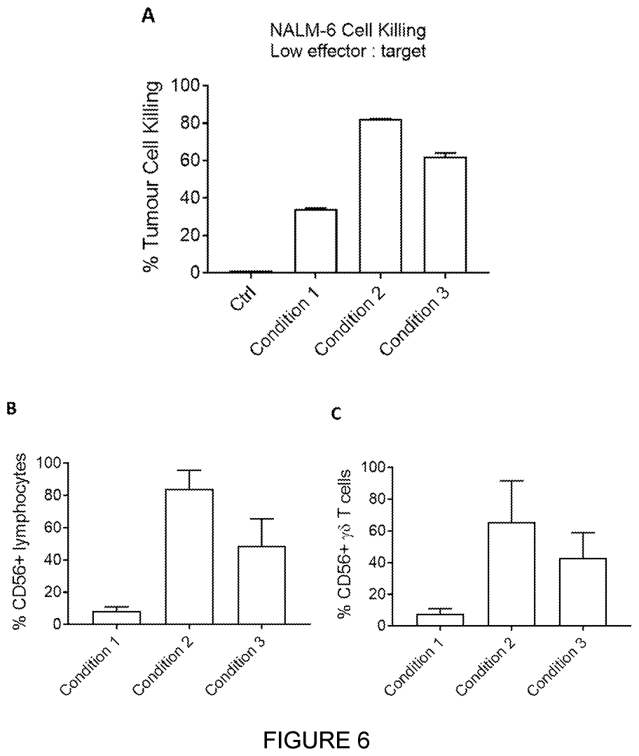

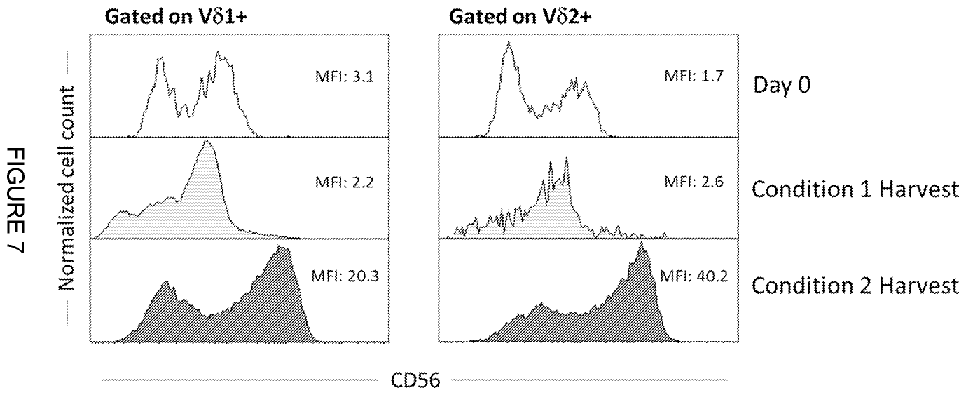

Cell compositions produced by conditions 1-3 were tested in a cytotoxicity assay as described in Example 1 using a low effector:target ratio of 1:1. Condition 2 showed the greatest cytotoxic activity, although conditions 1 and 3 both markedly augment cytotoxic activity towards NALM-6 tumour cells (FIG. 6A). The enhanced killing correlates with CD56 expression between the conditions (FIG. 68).

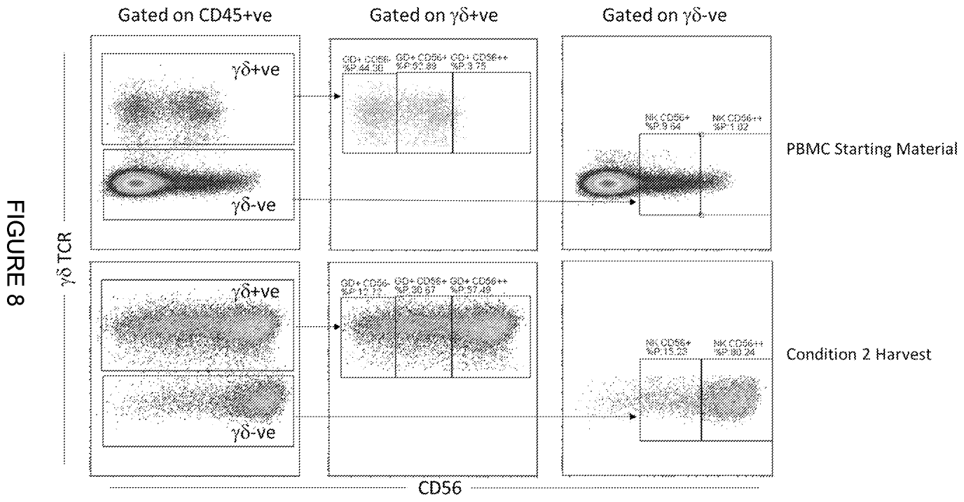

CD56 surface expression was also determined through analysis of the staining intensity via flow cytometry. Condition 2 strongly enriches the intensity of CD56 expression on Vδ1 and Vδ2 cells, relative to their intrinsic PBMC level (FIG. 7). Furthermore, increased abundance of CD56-bright cells is shown amongst both γδ and NK cell populations obtained from condition 2 harvest compared to initial PMBC starting material (FIG. 8).

### Example 5. Functionality of Cells after Storage

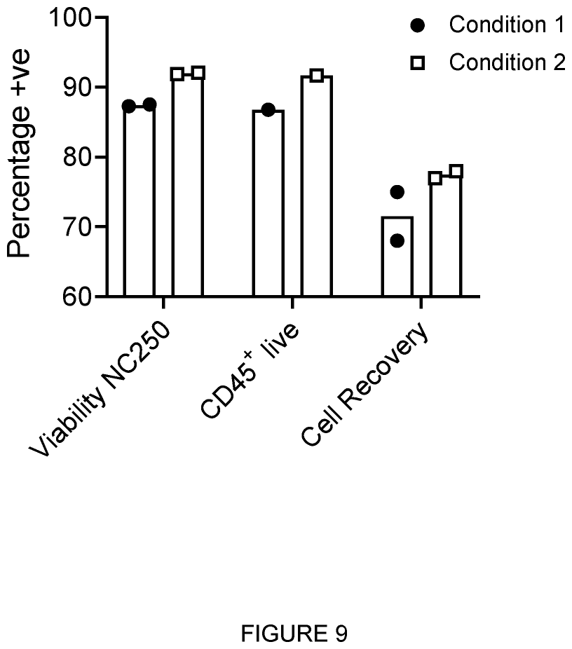

The functionality of cells after a storage step of freezing and then thawing was also investigated. A portion of cells cultured using conditions 1 and 2 were removed at days 11-14 and frozen. Cells were then thawed as described in Example 1. There is equivalent viability and recovery of cells between conditions 1 and 2 following cryopreservation (FIG. 9).

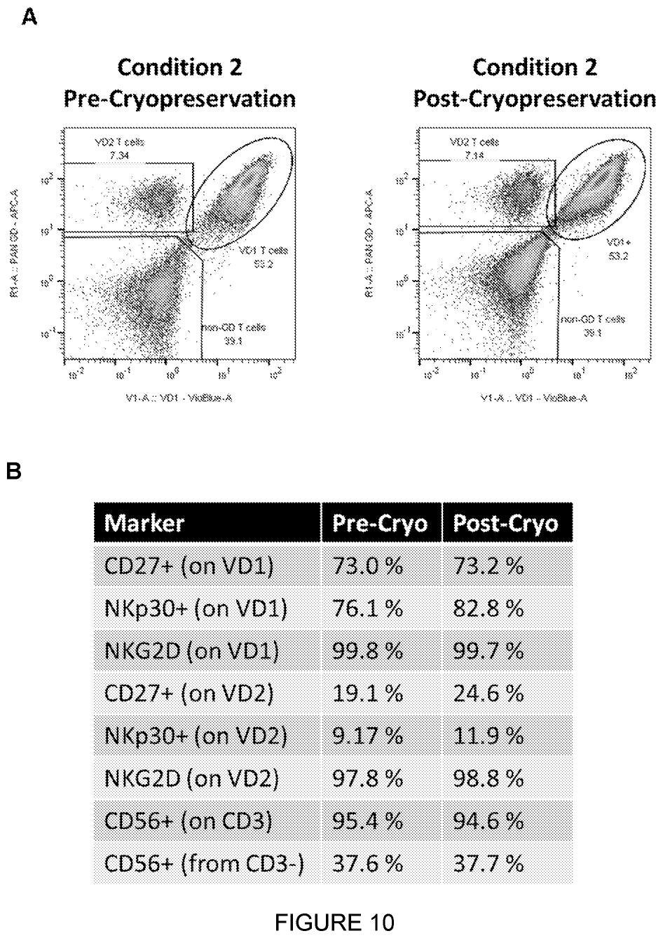

The phenotype of cells cryopreserved after expansion using condition 2 was also analysed. Condition 2 shows equivalent immune cell composition and cell surface phenotype pre- and post-cryopreservation (FIG. 10).

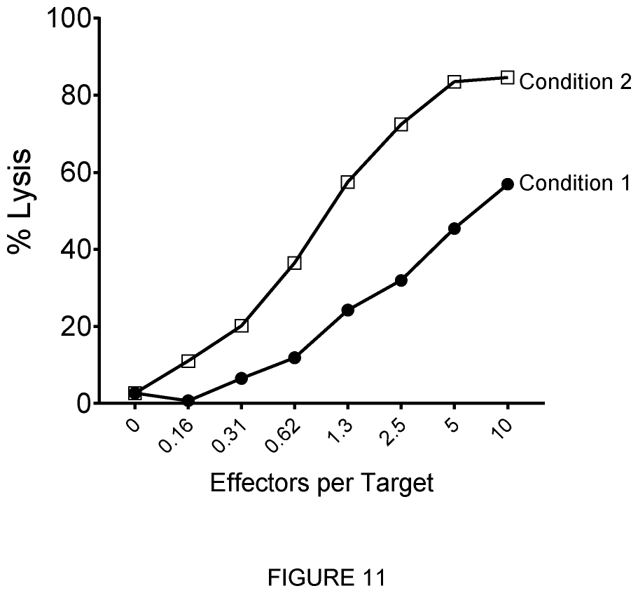

Finally, cytotoxicity of cells post-cryopreservation was investigated. Condition 2 shows enhanced cytotoxicity against NALM-6 tumour cells post cryopreservation across a range of effector to target ratios relative to condition 1 (FIG. 11).

### Example 6. Cell Composition Transduction

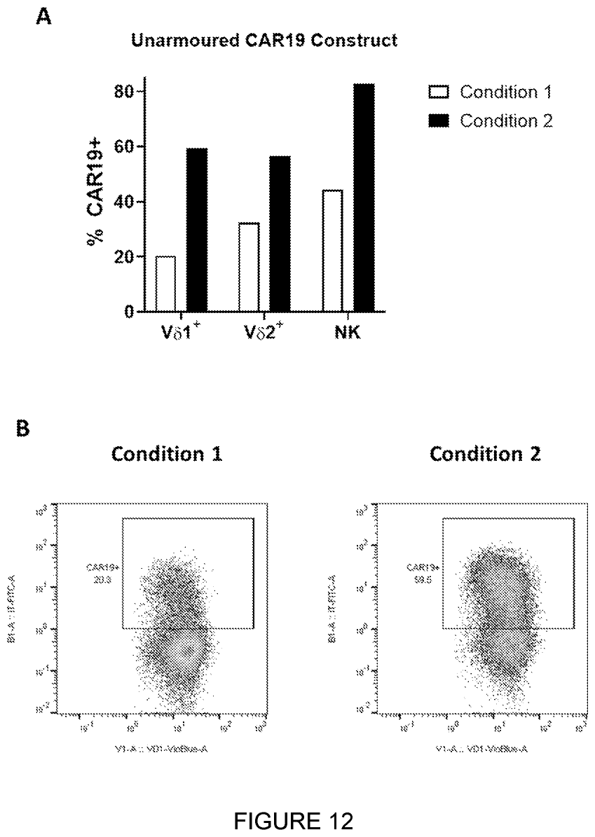

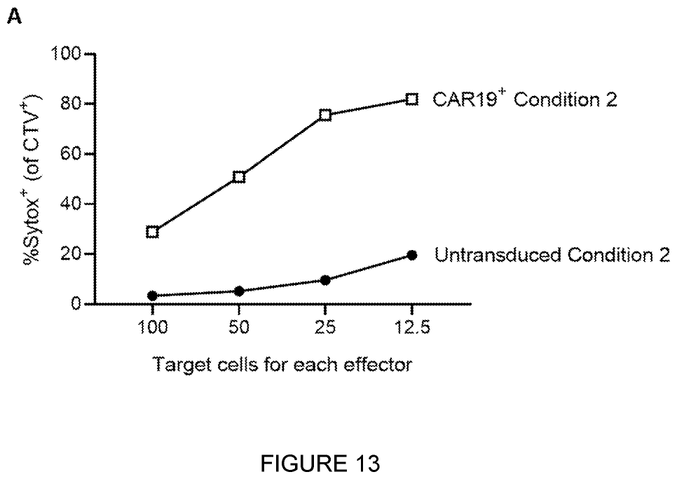

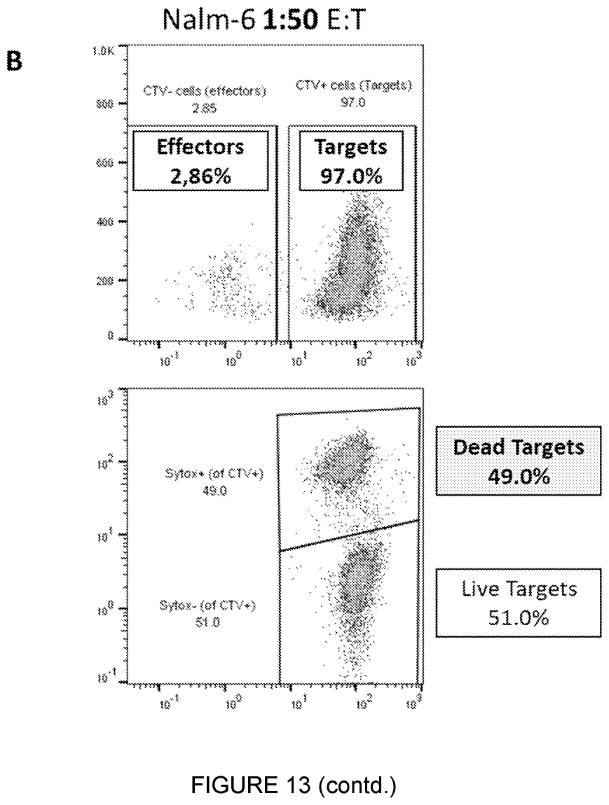

The cell composition obtained using condition 2 was transduced with a lentiviral vector as described in Example 1. Cells produced under condition 2 show enhanced permissibility to transduction relative to condition 1 (FIG. 12). Furthermore, CAR-transduced condition 2 enhances cytotoxicity towards NALM-6 cells further with killing observed at effector to target ratios as low as 1:100 (FIG. 13).

### Example 7. Optimised Method Conditions

The method was further optimized. CD56+ lymphocytes were generated from αβ-TCR+ depleted peripheral blood mononuclear cells as described in Condition 2 of Example 1 with the following changes. The culture media included 1×NK supplement (NK MACS®, Miltenyi Biotec). Unless otherwise specified, the soluble anti-Vδ1 monoclonal antibody was provided only at the beginning of culture and not further added. Both C08 and G04 in house soluble anti-Vδ1 monoclonal antibodies (described in WO2021032961) were tested with similar results, but unless specified, C08 was used. Cells were harvested at day 11 (12 day expansion) unless otherwise indicated based on the observation that Ki-67 expression, which indicates proliferative status, drops.

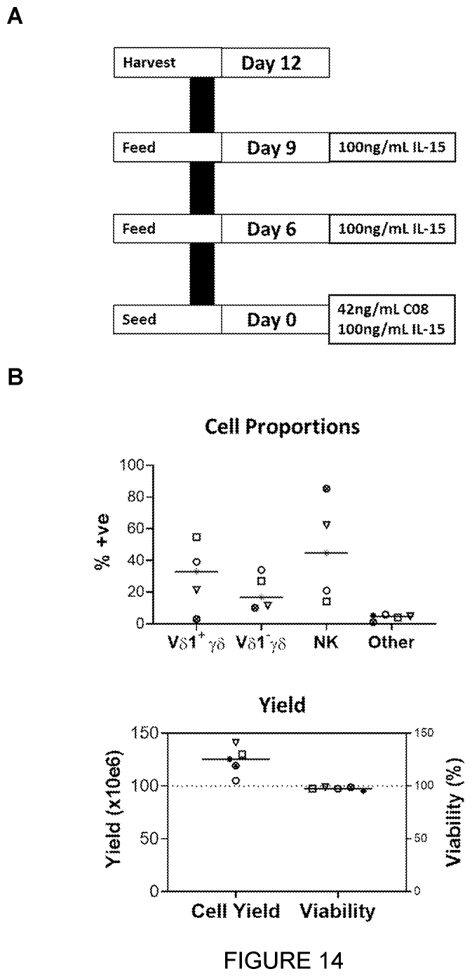

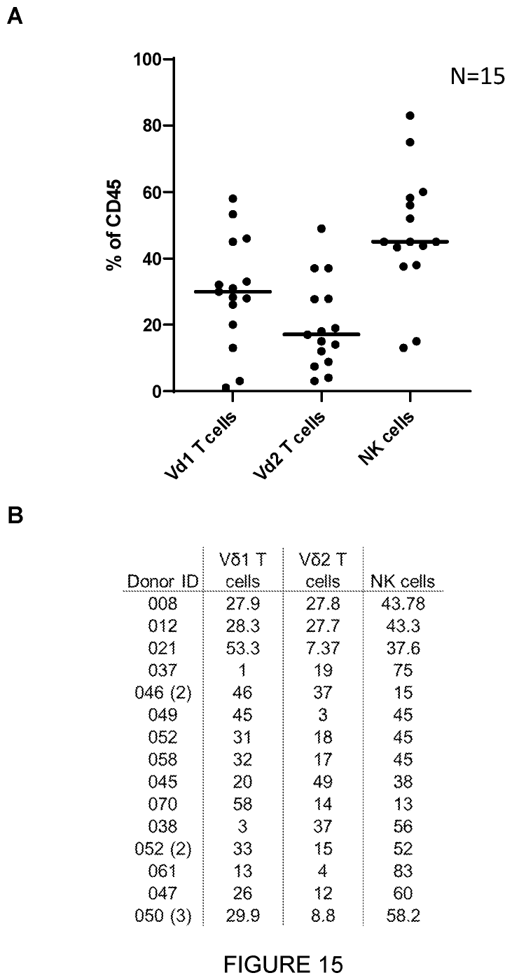

It was found that method is robust, with a shorter D11 harvest and single feed of the soluble anti-Vδ1 monoclonal antibody at day 0 provides good cell expansion with the correct cell types (FIG. 14). Repeat experiments using additional donors further demonstrate the robustness of the process (FIG. 15). Other parameters, such as culture volume, starting cell density, vessel type and addition of NK supplements either had no negative influence or had some positive effect (data not shown).

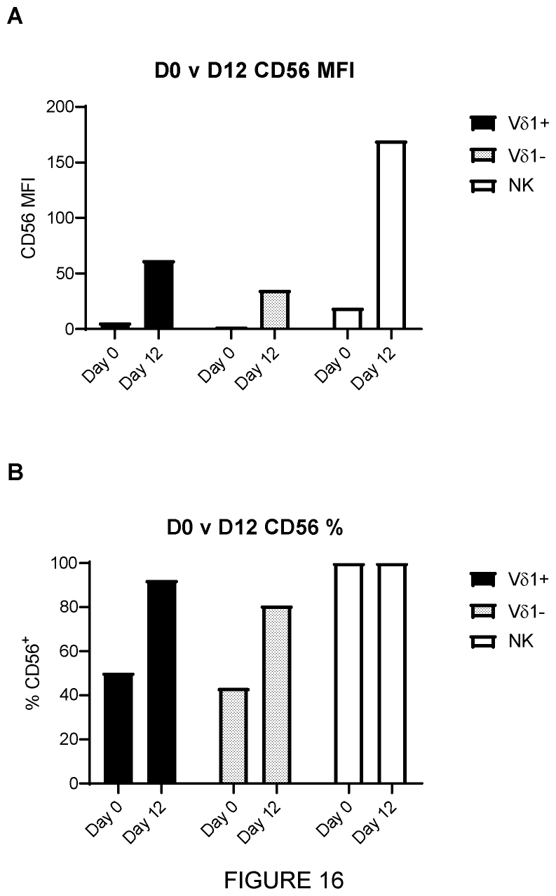

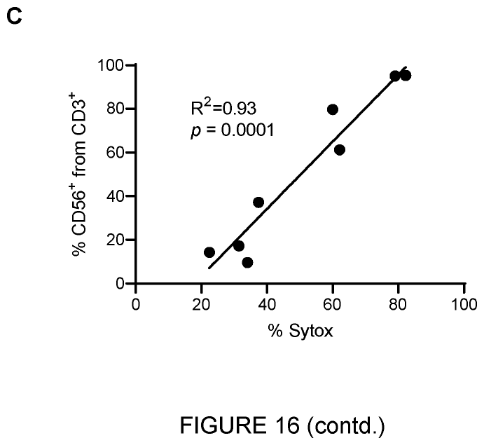

Furthermore, the cells from each cell type increased their expression of CD56, both in levels of expression (as shown by increased MFI, FIG. 16A), as well as percent positive for CD56 for the γδ cell fractions that are not normally high expressors of CD56 (FIG. 16B). Increased CD56 expression correlates with increased cytotoxicity (FIG. 16C).

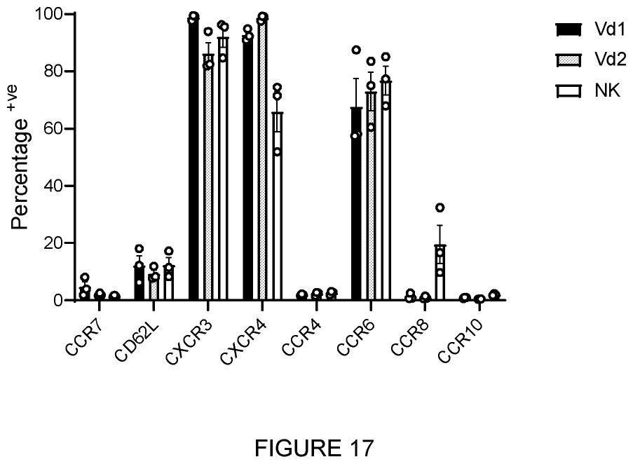

Finally, the expression levels of chemokine receptors on the surface of the cells are shown in FIG. 17. This indicates that these cells could potentially target a broad range of cell types.

### Example 8. Transduction with γ-Retroviral Vectors

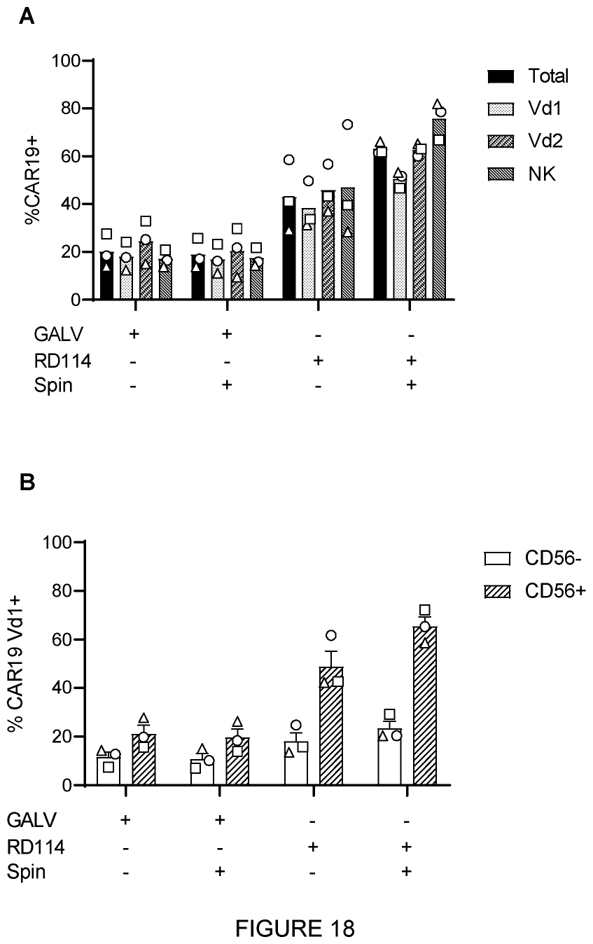

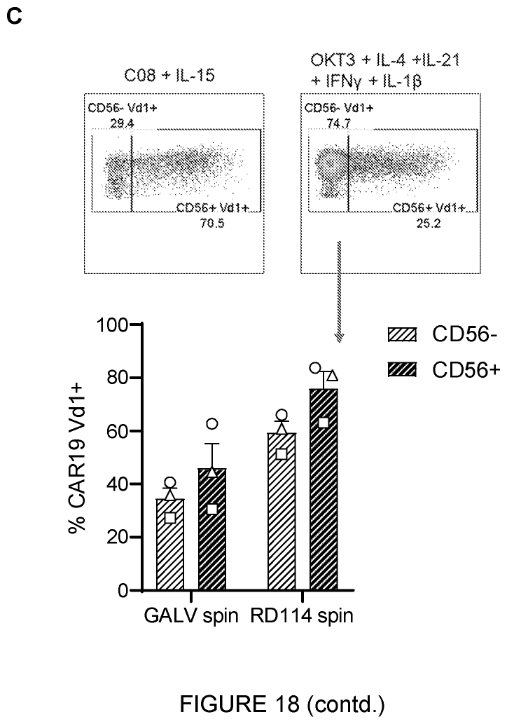

Cells were cultured as described in FIG. 14A. As a comparison, cells were seeded and cultured as described in Condition 2 of Example 1 with the following exception: soluble anti-CD3 monoclonal antibody (done OKT3) was used instead of soluble anti-Vδ1 monoclonal antibody. Cells were then transduced with GALV or RD114 pseudotyped γ-retrovirus vectors encoding CD19-targeting chimeric antigen receptors. Viral vector was diluted in CTS OpTmizer and adhered to RetroNectin coated (20 μg/mL) non tissue culture coated 6 well plate by centrifugation (2000×g for 2 h at 32° C.). Cultured cells were transferred to the virus preloaded plate. Cells were centrifuged at 800×g for 45 min at 32° C. (‘spin’) or left static (‘no spin’). The transduction efficiency was determined by flow cytometry 6 days post-transduction (FIG. 18A).

It was also shown that CD56+ cells of the present methods were much more transducible than the CD56− cells (FIG. 18B). This is unlike cells generated by the methods of WO2021032961, which was less affected by CD56 status (FIG. 18C).

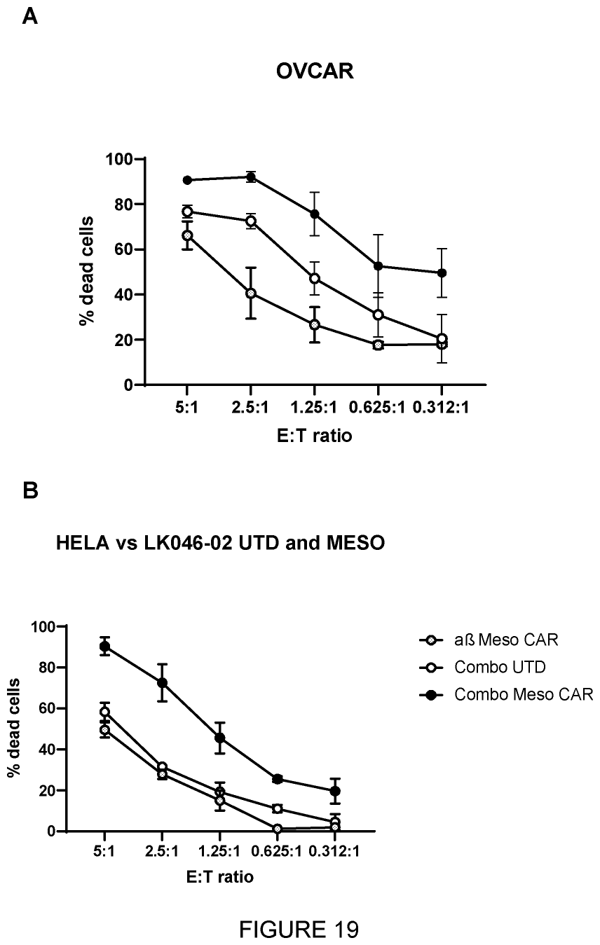

It has previously been shown that the cell composition, when transduced with a haem-targeted CAR (CD19), is able to kill haematological targets. FIG. 19 shows that the cell composition, when transduced with a mesothelin-targeting CAR can efficiently kill solid tumour targets. Indeed, the untransduced combination cell population of the present invention performs as well or better than the traditional CAR-expressing as T cells (lowest line), while the CAR expressing combination cell population substantially outperforms both.

1. An isolated composition comprising Natural Killer (NK) cells and γδ T cells wherein at least 40% of the γδ T cells present in the composition are CD56^{bright}.

2. An isolated composition comprising NK cells and γδ T cells wherein at least 50% of the γδ T cells present in the composition express CD56.

3. The isolated composition of claim 1 or claim 2, wherein at least 90% of the cells present in the composition consist of NK cells and γδ T cells.

4. The isolated composition of any one of claims 1 to 3, wherein at least 30% of the cells present in the composition are γδ T cells.

5. The isolated composition of any one of claims 1 to 4, wherein the γδ T cells comprise Vδ1 T cells and Vδ2 T cells.

6. The isolated composition of any one of claims 1 to 5, wherein at least 30% of the cells present in the composition are NK cells.

7. An isolated composition comprising cells wherein at least 90% of the cells consist of NK cells and γδ T cells, wherein at least 10% of the cells are Vδ1 T cells, at least 5% of the cells are Vδ2 T cells and at least 30% of the cells are NK cells.

8. The isolated composition of claim 7, wherein at least 40% of the γδ T cells present in the composition express CD56.

9. The isolated composition of any one of claims 1 to 8, wherein the γδ T cells comprise Vδ1 T cells and at least 15% of said Vδ1 T cells express NKp30.

10. The isolated composition of any one of claims 1 to 9, wherein the γδ T cells comprise Vδ1 T cells and less than 50% of said Vδ1 T cells express CD27.

11. The isolated composition of any one of claims 1 to 10, wherein the isolated composition comprises engineered NK cells and γδ T cells.

12. The isolated composition of any one of claims 1 to 11, for use in therapy.

13. The isolated composition of any one of claims 1 to 12, for use in a method of treating cancer, an infectious disease or an inflammatory disease.

14. A method of expanding non-γδ+ MHC unrestricted lymphocytes comprising stimulating a mixed cell population comprising γδ T cells and NK cells using an anti-TCR delta variable 1 (anti-Vδ1) antibody or fragment thereof, in the presence of Interleukin-15 (IL-15) and in the absence of Interleukin-4 (IL-4) and culturing the mixed cell population.

15. A method of preparing a composition comprising a cell population enriched for MHC unrestricted lymphocytes, wherein the method comprises:
(1) culturing a sample obtained from a subject in the presence of:
(i) an anti-Vδ1 antibody or fragment thereof; and
(ii) IL-15, in the absence of IL-4,
from the first day of said culturing; and
(2) isolating the cell population cultured from the sample.

16. The method of claim 15, wherein step (1) of the method comprises culturing the sample for at least 7 days, such as at least 14 days.

17. The method of claim 15 or claim 16, wherein at least 70% of the MHC unrestricted lymphocytes present in the cell population isolated in step (2) express CD56.

18. The method of any one of claims 15 to 17, wherein the cell population isolated in step (2) comprises γδT cells and at least 40% of the γδ T cells express CD56.

19. The method of any one of claims 15 to 18, wherein at least 90% of the cell population isolated in step (2) consists of NK cells and γδ T cells and wherein at least 10% of the cells are Vδ1 T cells, at least 5% of the cells are Vδ2 T cells and at least 30% of the cells are NK cells.

20. The method of any one of claims 14 to 19, wherein the activating anti-Vδ1 antibody or fragment thereof, binds to an epitope of a variable delta 1 (Vδ1) chain of a γδ T cell receptor (TCR) comprising one or more amino acid residues within amino acid regions:
(i) 3-20 of SEQ ID NO: 1; and/or
(ii) 37-77 of SEQ ID NO: 1.

21. The method of claim 20, wherein the epitope comprises one or more amino acid residues within amino acid regions: 5-20 and 62-77; 50-64; 37-53 and 59-72; 59-77; or 3-17 and 62-89, of SEQ ID NO: 1.

22. The method of any one of claims 14 to 21, wherein the anti-Vδ1 antibody or fragment thereof comprises one or more of:
a CDR3 comprising a sequence having at least 80% sequence identity with any one of SEQ ID NOs: 2-25;
a CDR2 comprising a sequence having at least 80% sequence identity with any one of SEQ ID NOs: 26-37 and SEQUENCES: A1-A12 (of Table 2); and/or
a CDR1 comprising a sequence having at least 80% sequence identity with any one of SEQ ID NOs: 38-61.

23. The method of any one of claims 15 to 22, wherein the sample is a haematopoietic sample or a fraction thereof.

24. The method of any one of claims 15 to 23, wherein the sample is depleted of αβ T cells prior to said culturing.

25. The method of any one of claims 15 to 24, wherein the culturing is performed in media comprising 2.5% plasma.

26. The method of any one of claims 15 to 25, wherein step (1) of the method comprises culturing the sample for about 12 days, such as 12 days.

27. A cell population enriched for MHC unrestricted lymphocytes obtainable by, such as obtained by, the method of any one of claims 14 to 26.

28. A composition comprising a cell population obtainable by, such as obtained by, the method of any one of claims 14 to 26.

29. The cell population of claim 27 or the composition of claim 28 for use in therapy.

30. The cell population of claim 27 or the composition of claim 28 for use in a method of treating cancer, an infectious disease or an inflammatory disease.


SEQUENCE LISTING (135 sequences)

SEQ ID NO: 1 - protein, 209 aa, Homo sapiens
AQKVTQAQSSVSMPVRKAVTLNCLYETSWWSYYIFWYKQLPSKEMIFLIRQGSDEQNAKSGRYSVNFKKAAKSVALTISALQLEDSAKYFCALGESLTRADKLIFGKGTRVTVEPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 2 - protein, 9 aa, Homo sapiens
VDYADAFDI

SEQ ID NO: 3 - protein, 9 aa, Homo sapiens
PIELGAFDI

SEQ ID NO: 4 - protein, 8 aa, Homo sapiens
TWSGYVDV

SEQ ID NO: 5 - protein, 9 aa, Homo sapiens
ENYLNAFDI

SEQ ID NO: 6 - protein, 8 aa, Homo sapiens
DSGVAFDI

SEQ ID NO: 7 - protein, 10 aa, Homo sapiens
HQVDTRTADY

SEQ ID NO: 8 - protein, 8 aa, Homo sapiens
SWNDAFDI

SEQ ID NO: 9 - protein, 8 aa, Homo sapiens
DYYYSMDV

SEQ ID NO: 10 - protein, 9 aa, Homo sapiens
HSWNDAFDV

SEQ ID NO: 11 - protein, 8 aa, Homo sapiens
DYYYSMDV

SEQ ID NO: 12 - protein, 9 aa, Homo sapiens
HSWSDAFDI

SEQ ID NO: 13 - protein, 9 aa, Homo sapiens
HSWNDAFDI

SEQ ID NO: 14 - protein, 9 aa, Homo sapiens
QQSYSTLLT

SEQ ID NO: 15 - protein, 11 aa, Homo sapiens
QVWDSSSDHVV

SEQ ID NO: 16 - protein, 10 aa, Homo sapiens
QQSYSTPQVT

SEQ ID NO: 17 - protein, 9 aa, Homo sapiens
QQSYSTPLT

SEQ ID NO: 18 - protein, 9 aa, Homo sapiens
QQFKRYPPT

SEQ ID NO: 19 - protein, 10 aa, Homo sapiens
SSYTSTSTLV

SEQ ID NO: 20 - protein, 9 aa, Homo sapiens
QQSYSTPLT

SEQ ID NO: 21 - protein, 9 aa, Homo sapiens
QQSHSHPPT

SEQ ID NO: 22 - protein, 9 aa, Homo sapiens
QQSYSTPDT

SEQ ID NO: 23 - protein, 9 aa, Homo sapiens
QQSYSTPVT

SEQ ID NO: 24 - protein, 9 aa, Homo sapiens
QQSYSTPLT

SEQ ID NO: 25 - protein, 9 aa, Homo sapiens
QQSYSTLLT

SEQ ID NO: 26 - protein, 8 aa, Homo sapiens
ISSSGSTI

SEQ ID NO: 27 - protein, 7 aa, Homo sapiens
IYSGGST

SEQ ID NO: 28 - protein, 9 aa, Homo sapiens
TYYRSKWST

SEQ ID NO: 29 - protein, 8 aa, Homo sapiens
ISSSGSTI

SEQ ID NO: 30 - protein, 8 aa, Homo sapiens
ISGGGGTT

SEQ ID NO: 31 - protein, 8 aa, Homo sapiens
IYPGDSDT

SEQ ID NO: 32 - protein, 9 aa, Homo sapiens
TYYRSKWYN

SEQ ID NO: 33 - protein, 9 aa, Homo sapiens
TYYRSKWYN

SEQ ID NO: 34 - protein, 8 aa, Homo sapiens
ISSSGSTI

SEQ ID NO: 35 - protein, 9 aa, Homo sapiens
TYYGSKWYN

SEQ ID NO: 36 - protein, 8 aa, Homo sapiens
ISSSGSTI

SEQ ID NO: 37 - protein, 8 aa, Homo sapiens
ISSSGSTI

SEQ ID NO: 38 - protein, 8 aa, Homo sapiens
GFTFSDYY

SEQ ID NO: 39 - protein, 8 aa, Homo sapiens
GFTVSSNY

SEQ ID NO: 40 - protein, 10 aa, Homo sapiens
GDSVSSKSAA

SEQ ID NO: 41 - protein, 8 aa, Homo sapiens
GFTFSDYY

SEQ ID NO: 42 - protein, 8 aa, Homo sapiens
GFTFSSYA

SEQ ID NO: 43 - protein, 8 aa, Homo sapiens
GYSFTSYW

SEQ ID NO: 44 - protein, 10 aa, Homo sapiens
GDSVSSNSAA

SEQ ID NO: 45 - protein, 10 aa, Homo sapiens
GDSVSSNSAA

SEQ ID NO: 46 - protein, 8 aa, Homo sapiens
GFTFSDYY

SEQ ID NO: 47 - protein, 10 aa, Homo sapiens
GDSVSSNSAA

SEQ ID NO: 48 - protein, 8 aa, Homo sapiens
GFTFSDYY

SEQ ID NO: 49 - protein, 8 aa, Homo sapiens
GFTFSDYY

SEQ ID NO: 50 - protein, 6 aa, Homo sapiens
QSIGTY

SEQ ID NO: 51 - protein, 6 aa, Homo sapiens
NIGSQS

SEQ ID NO: 52 - protein, 6 aa, Homo sapiens
QDINDW

SEQ ID NO: 53 - protein, 6 aa, Homo sapiens
QSLSNY

SEQ ID NO: 54 - protein, 6 aa, Homo sapiens
QNIRTW

SEQ ID NO: 55 - protein, 9 aa, Homo sapiens
RSDVGGYNY

SEQ ID NO: 56 - protein, 6 aa, Homo sapiens
QSISTW

SEQ ID NO: 57 - protein, 6 aa, Homo sapiens
QSISSW

SEQ ID NO: 58 - protein, 6 aa, Homo sapiens
QSISSY

SEQ ID NO: 59 - protein, 6 aa, Homo sapiens
QSISTW

SEQ ID NO: 60 - protein, 6 aa, Homo sapiens
QDISNY

SEQ ID NO: 61 - protein, 6 aa, Homo sapiens
QSISSH

SEQ ID NO: 62 - protein, 118 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVDYADAFDIWGQGTLVTVSS

SEQ ID NO: 63 - protein, 117 aa, Homo sapiens
EVQLLESGGGLVQPGRSLRLSCAASGFTVSSNYMSWVRQAPGKGLEWVSVIYSGGSTYYADSVKGRFTISRDNSKNTLYLQMNSLRAEDTAVYYCASPIELGAFDIWGQGTLVTVSS

SEQ ID NO: 64 - protein, 120 aa, Homo sapiens
QVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWSGYVDVWGQGTLVTVSS

SEQ ID NO: 65 - protein, 118 aa, Homo sapiens
EVQLLESGGGLVKPGGSLRLSCAASGFTFSDYYMSWIRQAPGKGLEWVSYISSSGSTIYYADSVKGRFTISRDNAKNSLYLQMNSLRAEDTAVYYCARENYLNAFDIWGRGTLVTVSS

SEQ ID NO: 66 - protein, 117 aa, Homo sapiens
EVQLLESGGGLVQPGGSLRLSCAASGFTFSSYAMSWVRQAPGKGLEWVSAISGGGGTTYSSDSVKGRFTISRDNSKNTLYLQMNSLRAEDTAVYYCARDSGVAFDIWGQGTLVTVSS

SEQ ID NO: 67 - protein, 119 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHQVDTRTADYWGQGTLVTVSS

SEQ ID NO: 68 - protein, 120 aa, Homo sapiens
QVQLQQSGPGLVKPSQTLSLTCAISGDSVSSNSAAWNWIRQSPSRGLEWLGRTYYRSKWYNDYAVSVRSRITINPDTSKNQFSLQLNSVTPEDTAVYYCARSWNDAFDIWGQGTTVTVSS

SEQ ID NO: 69 - protein, 120 aa, Homo sapiens
QVQLQQSGPGLVKPSQTLSLTCVISGDSVSSNSAAWNWIRQSPSRGLEWLGRTYYRSKWYNDYAVSVKSRITINPDTSKNQFSLQLNSVTPEDTAVYYCARDYYYSMDVWGQGTMVTVSS

SEQ ID NO: 70 - protein, 118 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARHSWNDAFDVWGQGTLVTVSS

SEQ ID NO: 71 - protein, 120 aa, Homo sapiens
QVQLQQSGPGLVKPSQTLSLTCAISGDSVSSNSAAWNWIRQSPSRGLEWLGRTYYGSKWYNEYALSVKSRIIINPDTSKNQFSLQLNSVTPEDTAVYYCARDYYYSMDVWGQGTLVTVSS

SEQ ID NO: 72 - protein, 118 aa, Homo sapiens
EVQLLESGGGLVKPGGSLRLSCAASGFTFSDYYMSWIRQAPGKGLEWVSYISSSGSTIYYADSVKGRFTISRDNAKNSLYLQMNSLRAEDTAVYYCARHSWSDAFDIWGQGTLVTVSS

SEQ ID NO: 73 - protein, 118 aa, Homo sapiens
EVQLVESGGGLVQPGGSLRLSCAASGFTFSDYYMSWIRQAPGKGLEWVSYISSSGSTIYYADSVKGRFTISRDNAKNSLYLQMNSLRAEDTAVYYCARHSWNDAFDIWGRGTLVTVSS

SEQ ID NO: 74 - protein, 109 aa, Homo sapiens
ASDIQMTQSPSSLSASVGDRVTIACRAGQSIGTYLNWYQQKPGKAPKLLIYVASSLQSGVPSRFSGSGSGTEFTLTISSLQPEDFATYYCQQSYSTLLTFGRGTKVEIK

SEQ ID NO: 75 - protein, 110 aa, Homo sapiens
ASSYELTQPPSVSVAPGKTARITCGGNNIGSQSVHWYQQKPGQAPMLVIYYDSDRPSGIPERFSGSNSGNTATLTISRVEAGDEADYYCQVWDSSSDHVVFGGGTKLTVL

SEQ ID NO: 76 - protein, 110 aa, Homo sapiens
ASDIQMTQSPPALSASVGDRVTITCRASQDINDWLAWYQHKPGKAPKLLIYDASSLESGVPSRFSGSGSGTEFTLTISSLQPDDFATYYCQQSYSTPQVTFGQGTRLEIK

SEQ ID NO: 77 - protein, 109 aa, Homo sapiens
ASDIQMTQSPSSLSASVGDRVTITCRTSQSLSNYLNWYQQKPGKAPKLLIYAASSLQSGVPSRFSGSGSGTDFTLTISSLQPEDFATYYCQQSYSTPLTFGGGTKLEIK

SEQ ID NO: 78 - protein, 109 aa, Homo sapiens
ASDIQMTQSPSFLSASVGDRVTITCRASQNIRTWLAWYQQKPGRAPKLLIYDASSLESGVPSRFSGSGSGTDFTLTISSLQPEDFATYYCQQFKRYPPTFGLGTKVEIK

SEQ ID NO: 79 - protein, 112 aa, Homo sapiens
ASQSALTQPASVSGSPGQSVTISCTGTRSDVGGYNYVSWYQHHPGKAPKLMIYEVSNRPSGVSNRFSGSKSGNTASLTISGLQAEDEADYYCSSYTSTSTLVFGGGTKLTVL

SEQ ID NO: 80 - protein, 109 aa, Homo sapiens
ASDIVMTQSPSTLSASIGDRVTITCRAGQSISTWLAWYQQKPGKAPKLLIYDASSLESGVPLRFSGSGSGTDFTLTISSLQPEDFATYYCQQSYSTPLTFGGGTKVEIK

SEQ ID NO: 81 - protein, 109 aa, Homo sapiens
ASDIQMTQSPSTLSASVGDRVTITCRASQSISSWLAWYQQKPGKAPKLLIYDASSLESGVPLRFSGSGSGTEFTLTISSLQPDDFATYYCQQSHSHPPTFGPGTKVDIK

SEQ ID NO: 82 - protein, 109 aa, Homo sapiens
ASDIQMTQSPSSLSASVGDRVSITCRASQSISSYLNWYQQKPGKAPKLLIYAASSLQSGVPSRFSGSGSGTDFTLTISSLQPEDFATYYCQQSYSTPDTFGGGTKVEIK

SEQ ID NO: 83 - protein, 109 aa, Homo sapiens
ASDIVMTQSPSTLSASIGDRVTITCRAGQSISTWLAWYQQKPGKAPKLLIYDASSLESGVPLRFSGSGSGTEFTLAISSLQPDDFATYYCQQSYSTPVTFGQGTKVEIK

SEQ ID NO: 84 - protein, 109 aa, Homo sapiens
ASDIQMTQSPSSLSASVGDRVTITCQASQDISNYLNWYQQKPGKAPKLLIYDASNLETGVPSRFSGSGSGTDFTLTISSLQPEDFATYYCQQSYSTPLTFGGGTKVEIK

SEQ ID NO: 85 - protein, 109 aa, Homo sapiens
ASDIQMTQSPSSLSASVGDRVTITCRASQSISSHLNWYQQKPGKAPKLLIYAASSLQSGVPSRFSASGSGTDFTLTISSLQPEDFATYYCQQSYSTLLTFGGGTKVEIK

SEQ ID NO: 86 - protein, 240 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVDYADAFDIWGQGTLVTVSSGGGGSGGGGSGGGASDIQMTQSPSSLSASVGDRVTIACRAGQSIGTYLNWYQQKPGKAPKLLIYVASSLQSGVPSRFSGSGSGTEFTLTISSLQPEDFATYYCQQSYSTLLTFGRGTKVEIK

SEQ ID NO: 87 - protein, 240 aa, Homo sapiens
EVQLLESGGGLVQPGRSLRLSCAASGFTVSSNYMSWVRQAPGKGLEWVSVIYSGGSTYYADSVKGRFTISRDNSKNTLYLQMNSLRAEDTAVYYCASPIELGAFDIWGQGTLVTVSSGGGGSGGGGSGGGASSYELTQPPSVSVAPGKTARITCGGNNIGSQSVHWYQQKPGQAPMLVIYYDSDRPSGIPERFSGSNSGNTATLTISRVEAGDEADYYCQVWDSSSDHVVFGGGTKLTVL

SEQ ID NO: 88 - protein, 243 aa, Homo sapiens
QVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWSGYVDVWGQGTLVTVSSGGGGSGGGGSGGGASDIQMTQSPPALSASVGDRVTITCRASQDINDWLAWYQHKPGKAPKLLIYDASSLESGVPSRFSGSGSGTEFTLTISSLQPDDFATYYCQQSYSTPQVTFGQGTRLEIK

SEQ ID NO: 89 - protein, 240 aa, Homo sapiens
EVQLLESGGGLVKPGGSLRLSCAASGFTFSDYYMSWIRQAPGKGLEWVSYISSSGSTIYYADSVKGRFTISRDNAKNSLYLQMNSLRAEDTAVYYCARENYLNAFDIWGRGTLVTVSSGGGGSGGGGSGGGASDIQMTQSPSSLSASVGDRVTITCRTSQSLSNYLNWYQQKPGKAPKLLIYAASSLQSGVPSRFSGSGSGTDFTLTISSLQPEDFATYYCQQSYSTPLTFGGGTKLEIK

SEQ ID NO: 90 - protein, 239 aa, Homo sapiens
EVQLLESGGGLVQPGGSLRLSCAASGFTFSSYAMSWVRQAPGKGLEWVSAISGGGGTTYSSDSVKGRFTISRDNSKNTLYLQMNSLRAEDTAVYYCARDSGVAFDIWGQGTLVTVSSGGGGSGGGGSGGGASDIQMTQSPSFLSASVGDRVTITCRASQNIRTWLAWYQQKPGRAPKLLIYDASSLESGVPSRFSGSGSGTDFTLTISSLQPEDFATYYCQQFKRYPPTFGLGTKVEIK

SEQ ID NO: 91 - protein, 244 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHQVDTRTADYWGQGTLVTVSSGGGGSGGGGSGGGASQSALTQPASVSGSPGQSVTISCTGTRSDVGGYNYVSWYQHHPGKAPKLMIYEVSNRPSGVSNRFSGSKSGNTASLTISGLQAEDEADYYCSSYTSTSTLVFGGGTKLTVL

SEQ ID NO: 92 - protein, 242 aa, Homo sapiens
QVQLQQSGPGLVKPSQTLSLTCAISGDSVSSNSAAWNWIRQSPSRGLEWLGRTYYRSKWYNDYAVSVRSRITINPDTSKNQFSLQLNSVTPEDTAVYYCARSWNDAFDIWGQGTTVTVSSGGGGSGGGGSGGGASDIVMTQSPSTLSASIGDRVTITCRAGQSISTWLAWYQQKPGKAPKLLIYDASSLESGVPLRFSGSGSGTDFTLTISSLQPEDFATYYCQQSYSTPLTFGGGTKVEIK

SEQ ID NO: 93 - protein, 242 aa, Homo sapiens
QVQLQQSGPGLVKPSQTLSLTCVISGDSVSSNSAAWNWIRQSPSRGLEWLGRTYYRSKWYNDYAVSVKSRITINPDTSKNQFSLQLNSVTPEDTAVYYCARDYYYSMDVWGQGTMVTVSSGGGGSGGGGSGGGASDIQMTQSPSTLSASVGDRVTITCRASQSISSWLAWYQQKPGKAPKLLIYDASSLESGVPLRFSGSGSGTEFTLTISSLQPDDFATYYCQQSHSHPPTFGPGTKVDIK

SEQ ID NO: 94 - protein, 240 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARHSWNDAFDVWGQGTLVTVSSGGGGSGGGGSGGGASDIQMTQSPSSLSASVGDRVSITCRASQSISSYLNWYQQKPGKAPKLLIYAASSLQSGVPSRFSGSGSGTDFTLTISSLQPEDFATYYCQQSYSTPDTFGGGTKVEIK

SEQ ID NO: 95 - protein, 242 aa, Homo sapiens
QVQLQQSGPGLVKPSQTLSLTCAISGDSVSSNSAAWNWIRQSPSRGLEWLGRTYYGSKWYNEYALSVKSRIIINPDTSKNQFSLQLNSVTPEDTAVYYCARDYYYSMDVWGQGTLVTVSSGGGGSGGGGSGGGASDIVMTQSPSTLSASIGDRVTITCRAGQSISTWLAWYQQKPGKAPKLLIYDASSLESGVPLRFSGSGSGTEFTLAISSLQPDDFATYYCQQSYSTPVTFGQGTKVEIK

SEQ ID NO: 96 - protein, 240 aa, Homo sapiens
EVQLLESGGGLVKPGGSLRLSCAASGFTFSDYYMSWIRQAPGKGLEWVSYISSSGSTIYYADSVKGRFTISRDNAKNSLYLQMNSLRAEDTAVYYCARHSWSDAFDIWGQGTLVTVSSGGGGSGGGGSGGGASDIQMTQSPSSLSASVGDRVTITCQASQDISNYLNWYQQKPGKAPKLLIYDASNLETGVPSRFSGSGSGTDFTLTISSLQPEDFATYYCQQSYSTPLTFGGGTKVEIK

SEQ ID NO: 97 - protein, 240 aa, Homo sapiens
EVQLVESGGGLVQPGGSLRLSCAASGFTFSDYYMSWIRQAPGKGLEWVSYISSSGSTIYYADSVKGRFTISRDNAKNSLYLQMNSLRAEDTAVYYCARHSWNDAFDIWGRGTLVTVSSGGGGSGGGGSGGGASDIQMTQSPSSLSASVGDRVTITCRASQSISSHLNWYQQKPGKAPKLLIYAASSLQSGVPSRFSASGSGTDFTLTISSLQPEDFATYYCQQSYSTLLTFGGGTKVEIK

SEQ ID NO: 98 - protein, 13 aa, Artificial Sequence
  Synthetic linker
GGGGSGGGGSGGG

SEQ ID NO: 99 - DNA, 765 bp, Homo sapiens
gccatggcccaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggctgtgtattactgtgcaagggtggactacgctgatgcatttgatatctggggccagggcaccctggtcaccgtctcgagtggtggaggcggttcaggcggaggtggctctggcggtggcgctagcgacatccagatgacccagtctccatcctccctgtctgcatctgtaggagacagagtcaccatcgcttgccgggcaggtcagagcattggcacctatttaaattggtatcagcagaaaccagggaaagcccctaaactcctgatctatgttgcatccagtttgcaaagtggggtcccgtcacggttcagtggcagtggatctgggacagaattcactctcaccatcagcagtctgcaacctgaagattttgcaacttactactgtcaacagagttacagtaccctcctcactttcggcagagggaccaaggtggaaatcaaacgtaccgcggccgcatccgcacatcatcatcaccat

SEQ ID NO: 100 - DNA, 768 bp, Homo sapiens
gccatggccgaggtgcagctgttggagtctgggggaggcttggtacagcctggcaggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggctgtgtattactgtgcgagccccatagagctgggtgcttttgatatctggggccaaggaaccctggtcaccgtctcgagtggtggaggcggttcaggcggaggtggctctggcggtggcgctagctcctatgagctgactcagccaccctcagtgtcagtggccccaggaaagacggccaggattacctgtgggggaaacaacattggaagtcaaagtgtgcactggtaccagcagaagccaggccaggcccctatgctggtcatctattatgatagcgaccggccctcagggatccctgagcgattctctggctccaactctgggaacacggccaccctgaccatcagcagggtcgaagccggggatgaggccgactattactgtcaggtgtgggatagtagtagtgatcatgtggtattcggcggcgggaccaagctgaccgtcctaggtcagcccgcggccgcatccgcacatcatcatcaccat

SEQ ID NO: 101 - DNA, 774 bp, Homo sapiens
gccatggcccaggtacagctgcagcagtcaggtccaggactggtgaagccctcgcagaccctctcactcacctgtgccatctccggggacagtgtctccagcaaaagtgctgcttggaactggatcaggcagtccccatcgagaggccttgagtggctgggaaggacatactacaggtccaagtggtctactgattatgcagcatctgtgaaaagtcgaataaccatcaacccagacacatccaagaaccagctctccctgcagttaaactctgtgactcccgaggacacggctgtgtattactgtgcaagaacgtggagtggttatgtggacgtctggggccaaggaaccctggtcaccgtctcgagtggtggaggcggttcaggcggaggtggctctggcggtggcgctagcgacatccagatgacccagtcccctcccgccctgtctgcatctgtgggagacagagtcaccatcacttgccgggccagtcaagatattaatgactggttggcctggtatcagcataaacctgggaaagcccctaagctcctgatctatgatgcctccagtttggaaagtggggtcccatcaaggttcagcggcagtggatctgggacagaattcactctcaccatcagcagcctgcagcctgatgattttgcaacttactactgtcaacagagttacagtacccctcaggtcacttttggccaggggacacgactggagatcaaacgtaccgcggccgcatccgcacatcatcatcaccat

SEQ ID NO: 102 - DNA, 765 bp, Homo sapiens
gccatggccgaggtgcagctgttggagtctgggggaggcttggtcaagcctggagggtccctgagactctcctgtgcggcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagagaaaactatctaaatgcttttgatatctggggccgtggcaccctggtcaccgtctcgagtggtggaggcggttcaggcggaggtggctctggcggtggcgctagcgacatccagatgacccagtctccatcctccctgtctgcatctgtaggagacagagtcaccatcacttgccggacaagtcagagccttagtaattacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcgacttactactgtcaacagagttacagtacccctctcactttcggcggagggaccaagctagagatcaaacgtaccgcggccgcatccgcacatcatcatcaccat

SEQ ID NO: 103 - DNA, 762 bp, Homo sapiens
gccatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtggtggtggtaccacatactcctcagactccgtgaagggccggttcaccatctccagagacaattccaagaacacgctgtatctgcaaatgaacagcctgagagccgaggacacggctgtgtattactgtgcgagagattcaggggttgcttttgatatctggggccaaggaaccctggtcaccgtctcgagtggtggaggcggttcaggcggaggtggctctggcggtggcgctagcgacatccagatgacccagtctccatccttcctgtctgcatctgtaggagacagagtcaccatcacttgccgggccagtcagaatatacgtacctggttggcctggtatcagcagaaaccagggagagcccctaagctcctgatctatgatgcctccagtttggaaagtggggtcccatcaaggttcagcggcagtggatctgggactgatttcactctcaccatcagcagcctgcagcctgaagattttgcaacttattactgtcaacagtttaaacgttaccctccgacgtttggcctggggaccaaggtggagatcaaacgtaccgcggccgcatccgcacatcatcatcaccat

SEQ ID NO: 104 - DNA, 780 bp, Homo sapiens
gccatggcccaggtccagctggtacagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattattgtgcgagacatcaggttgatacacggacggctgattactggggccagggaaccctggtcaccgtctcgagtggtggaggcggttcaggcggaggtggctctggcggtggcgctagccagtctgcgctgactcagcctgcctccgtgtctgggtctcctggacagtcggtcaccatctcctgcactggaaccaggagtgacgttggtggttataactatgtctcctggtaccaacaccacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcaccagcactctggtattcggcggagggaccaagctgaccgtcctaggtcagcccgcggccgcatccgcacatcatcatcaccat

SEQ ID NO: 105 - DNA, 771 bp, Homo sapiens
gccatggcccaggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgagaagtcgaataaccatcaacccagacacatccaagaaccagttctccctgcagctgaactctgtgactcccgaggacacggctgtgtattactgtgcaagaagctggaatgatgcttttgatatctgggggcaagggaccacggtcaccgtctcgagtggtggaggcggttcaggcggaggtggctctggcggtggcgctagcgatattgtgatgacacagtctccttccaccctgtctgcatctataggagacagagtcaccatcacttgccgggccggtcagagtattagtacctggttggcctggtatcagcagaaaccagggaaagcccctaagctcctgatctatgatgcctccagtttggaaagtggggtcccattaaggttcagcggcagtggatctgggacagatttcactctcaccatcagcagtctgcaacctgaagattttgcaacttactactgtcaacagagttacagtaccccgctcactttcggcggagggaccaaggtggagatcaaacgtaccgcggccgcatccgcacatcatcatcaccat

SEQ ID NO: 106 - DNA, 771 bp, Homo sapiens
gccatggcccaggtacagctgcagcagtcaggtccaggcctggtgaagccctcgcagaccctctcactcacctgtgtcatctccggggacagtgtctctagcaacagtgctgcttggaactggatcaggcagtccccatcgcgaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtctattattgtgcaagagactactactacagtatggacgtctggggccaagggacaatggtcaccgtctcgagtggtggaggcggttcaggcggaggtggctctggcggtggcgctagcgacatccagatgacccagtctccctccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccattaaggttcagcggcagtggatctgggacagaattcactctcaccatcagcagcctgcagcctgatgattttgcaacttattactgtcaacagagtcacagtcacccccctactttcggccctgggaccaaagtggatatcaaacgtaccgcggccgcatccgcacatcatcatcaccat

SEQ ID NO: 107 - DNA, 765 bp, Homo sapiens
gccatggcccaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagacatagctggaatgatgcttttgatgtctggggccagggaaccctggtcaccgtctcgagtggtggaggcggttcaggcggaggtggctctggcggtggcgctagcgacatccagatgacccagtctccatcctccctgtctgcatctgtaggagacagagtctccatcacctgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcagagttacagtacccccgacactttcggcggagggaccaaggtggaaatcaaacgtaccgcggccgcatccgcacatcatcatcaccat

SEQ ID NO: 108 - DNA, 771 bp, Homo sapiens
gccatggcccaggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacgggtccaagtggtataatgagtatgcactatctgtgaaaagtcgaataatcatcaacccagacacatccaagaaccagttctccctgcagctgaactctgtgactcccgaggacacggctgtctattattgtgcaagagactactactacagtatggacgtctggggccagggaaccctggtcaccgtctcgagtggtggaggcggttcaggcggaggtggctctggcggtggcgctagcgatattgtgatgactcagtctccttccaccctgtctgcatctataggagacagagtcaccatcacttgccgggccggtcagagtattagtacctggttggcctggtatcagcagaaaccagggaaagcccctaagctcctgatctatgatgcctccagtttggaaagtggggtcccattaaggttcagcggcagtggatctgggacagaattcactctcgccatcagcagcctgcagcctgatgattttgcaacttactactgtcaacagagttacagtaccccggtaacttttggccaggggaccaaggtggagatcaaacgtaccgcggccgcatccgcacatcatcatcaccat

SEQ ID NO: 109 - DNA, 765 bp, Homo sapiens
gccatggccgaggtgcagctgttggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagacatagctggagtgatgcttttgatatctggggccaaggaaccctggtcaccgtctcgagtggtggaggcggttcaggcggaggtggctctggcggtggcgctagcgacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagatttcactctcaccatcagcagtctgcaacctgaagattttgcaacttactactgtcaacagagttacagtactcctctcactttcggcggagggaccaaggtggagatcaaacgtaccgcggccgcatccgcacatcatcatcaccat

SEQ ID NO: 110 - DNA, 765 bp, Homo sapiens
gccatggccgaagtgcagctggtggagtccgggggaggcttagttcagcctggggggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagacatagctggaatgatgcttttgatatctggggccgtggcaccctggtcaccgtctcgagtggtggaggcggttcaggcggaggtggctctggcggtggcgctagcgacatccagatgacccagtctccatcctccctgtctgcatctgtaggagacagagtcaccatcacttgccgggcaagtcagagcattagcagccatttaaattggtatcagcagaaaccagggaaagcccctaagctcctgatctatgctgcatccagtttgcaaagtggggtcccatccaggttcagtgccagtggatctgggacagatttcactctcaccatcagcagcctgcagcctgaagattttgcaacttactactgtcaacagagttacagtaccctgctcactttcggcggagggaccaaggtggaaatcaaacgtaccgcggccgcatccgcacatcatcatcaccat

SEQ ID NO: 111 - protein, 664 aa, Homo sapiens
ASDIQMTQSPSSLSASVGDRVTIACRAGQSIGTYLNWYQQKPGKAPKLLIYVASSLQSGVPSRFSGSGSGTEFTLTISSLQPEDFATYYCQQSYSTLLTFGRGTKVEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVDYADAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 667 aa, Homo sapiens
ASDIQMTQSPPALSASVGDRVTITCRASQDINDWLAWYQHKPGKAPKLLIYDASSLESGVPSRFSGSGSGTEFTLTISSLQPDDFATYYCQQSYSTPQVTFGQGTRLEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECQVQLQQSGPGLVKPSQTLSLTCAISGDSVSSKSAAWNWIRQSPSRGLEWLGRTYYRSKWSTDYAASVKSRITINPDTSKNQLSLQLNSVTPEDTAVYYCARTWSGYVDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 664 aa, Homo sapiens
ASDIQMTQSPSSLSASVGDRVTITCRTSQSLSNYLNWYQQKPGKAPKLLIYAASSLQSGVPSRFSGSGSGTDFTLTISSLQPEDFATYYCQQSYSTPLTFGGGTKLEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECEVQLLESGGGLVKPGGSLRLSCAASGFTFSDYYMSWIRQAPGKGLEWVSYISSSGSTIYYADSVKGRFTISRDNAKNSLYLQMNSLRAEDTAVYYCARENYLNAFDIWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 663 aa, Homo sapiens
ASDIQMTQSPSFLSASVGDRVTITCRASQNIRTWLAWYQQKPGRAPKLLIYDASSLESGVPSRFSGSGSGTDFTLTISSLQPEDFATYYCQQFKRYPPTFGLGTKVEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECEVQLLESGGGLVQPGGSLRLSCAASGFTFSSYAMSWVRQAPGKGLEWVSAISGGGGTTYSSDSVKGRFTISRDNSKNTLYLQMNSLRAEDTAVYYCARDSGVAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 667 aa, Homo sapiens
ASQSALTQPASVSGSPGQSVTISCTGTRSDVGGYNYVSWYQHHPGKAPKLMIYEVSNRPSGVSNRFSGSKSGNTASLTISGLQAEDEADYYCSSYTSTSTLVFGGGTKLTVLGQPAAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSQVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHQVDTRTADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 663 aa, Homo sapiens
ASSYELTQPPSVSVAPGKTARITCGGNNIGSQSVHWYQQKPGQAPMLVIYYDSDRPSGIPERFSGSNSGNTATLTISRVEAGDEADYYCQVWDSSSDHVVFGGGTKLTVLGQPAAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSEVQLLESGGGLVQPGRSLRLSCAASGFTVSSNYMSWVRQAPGKGLEWVSVIYSGGSTYYADSVKGRFTISRDNSKNTLYLQMNSLRAEDTAVYYCASPIELGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 666 aa, Homo sapiens
ASDIVMTQSPSTLSASIGDRVTITCRAGQSISTWLAWYQQKPGKAPKLLIYDASSLESGVPLRFSGSGSGTDFTLTISSLQPEDFATYYCQQSYSTPLTFGGGTKVEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECQVQLQQSGPGLVKPSQTLSLTCAISGDSVSSNSAAWNWIRQSPSRGLEWLGRTYYRSKWYNDYAVSVRSRITINPDTSKNQFSLQLNSVTPEDTAVYYCARSWNDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 666 aa, Homo sapiens
ASDIQMTQSPSTLSASVGDRVTITCRASQSISSWLAWYQQKPGKAPKLLIYDASSLESGVPLRFSGSGSGTEFTLTISSLQPDDFATYYCQQSHSHPPTFGPGTKVDIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECQVQLQQSGPGLVKPSQTLSLTCVISGDSVSSNSAAWNWIRQSPSRGLEWLGRTYYRSKWYNDYAVSVKSRITINPDTSKNQFSLQLNSVTPEDTAVYYCARDYYYSMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 664 aa, Homo sapiens
ASDIQMTQSPSSLSASVGDRVSITCRASQSISSYLNWYQQKPGKAPKLLIYAASSLQSGVPSRFSGSGSGTDFTLTISSLQPEDFATYYCQQSYSTPDTFGGGTKVEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARHSWNDAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 666 aa, Homo sapiens
ASDIVMTQSPSTLSASIGDRVTITCRAGQSISTWLAWYQQKPGKAPKLLIYDASSLESGVPLRFSGSGSGTEFTLAISSLQPDDFATYYCQQSYSTPVTFGQGTKVEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECQVQLQQSGPGLVKPSQTLSLTCAISGDSVSSNSAAWNWIRQSPSRGLEWLGRTYYGSKWYNEYALSVKSRIIINPDTSKNQFSLQLNSVTPEDTAVYYCARDYYYSMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 664 aa, Homo sapiens
ASDIQMTQSPSSLSASVGDRVTITCQASQDISNYLNWYQQKPGKAPKLLIYDASNLETGVPSRFSGSGSGTDFTLTISSLQPEDFATYYCQQSYSTPLTFGGGTKVEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECEVQLLESGGGLVKPGGSLRLSCAASGFTFSDYYMSWIRQAPGKGLEWVSYISSSGSTIYYADSVKGRFTISRDNAKNSLYLQMNSLRAEDTAVYYCARHSWSDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 664 aa, Homo sapiens
ASDIQMTQSPSSLSASVGDRVTITCRASQSISSHLNWYQQKPGKAPKLLIYAASSLQSGVPSRFSASGSGTDFTLTISSLQPEDFATYYCQQSYSTLLTFGGGTKVEIKRTAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECEVQLVESGGGLVQPGGSLRLSCAASGFTFSDYYMSWIRQAPGKGLEWVSYISSSGSTIYYADSVKGRFTISRDNAKNSLYLQMNSLRAEDTAVYYCARHSWNDAFDIWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 254 aa, Artificial Sequence
  TRDV1(3OMZ) TRAC antigen sequence
AQKVTQAQSSVSMPVRKAVTLNCLYETSWWSYYIFWYKQLPSKEMIFLIRQGSDEQNAKSGRYSVNFKKAAKSVALTISALQLEDSAKYFCALGESLTRADKLIFGKGTRVTVEPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCTTAPSAQLKKKLQALKKKNAQLKWKLQALKKKLAQGSGHHHHHH

SEQ ID NO: 124 - protein, 12 aa, Artificial Sequence
  Optional scFv tag sequence (kappa sequence + His tag)
RTAAASAHHHHH

SEQ ID NO: 125 - protein, 37 aa, Artificial Sequence
  Optional scFv tag sequence (kappa sequence + His and FLAG tag)
RTAAASAHHHHHHKLDYKDHDGDYKDHDIDYKDDDDK

SEQ ID NO: 126 - protein, 13 aa, Artificial Sequence
  Optional scFv tag sequence (lambda sequence + His tag)
GQPAAASAHHHHH

SEQ ID NO: 127 - protein, 38 aa, Artificial Sequence
  Optional scFv tag sequence (lambda sequence + His and FLAG tag)
GQPAAASAHHHHHHKLDYKDHDGDYKDHDIDYKDDDDK

SEQ ID NO: 128 - protein, 254 aa, Homo sapiens
AQKVTQAQSSVSMPVRKAVTLNCLYETSWWSYYIFWYKQLPSKEMIFLIRQGSDEQNAKSGRYSVNFKKAVKSVALTISALQLEDSAKYFCALGESLTRADKLIFGKGTRVTVEPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCTTAPSAQLKKKLQALKKKNAQLKWKLQALKKKLAQGSGHHHHHH

SEQ ID NO: 129 - protein, 20 aa, Homo sapiens
MLFSSLLCVFVAFSYSGSSV

SEQ ID NO: 130 - protein, 95 aa, Homo sapiens
AQKVTQAQSSVSMPVRKAVTLNCLYETSWWSYYIFWYKQLPSKEMIFLIRQGSDEQNAKSGRYSVNFKKAAKSVALTISALQLEDSAKYFCALGE

SEQ ID NO: 131 - protein, 16 aa, Homo sapiens
TDKLIFGKGTRVTVEP

SEQ ID NO: 132 - protein, 17 aa, Homo sapiens
LTAQLFFGKGTQLIVEP

SEQ ID NO: 133 - protein, 19 aa, Homo sapiens
SWDTRQMFFGTGIKLFVEP

SEQ ID NO: 134 - protein, 15 aa, Homo sapiens
RPLIFGKGTYLEVQQ

SEQ ID NO: 135 - protein, 153 aa, Homo sapiens
  misc_feature (1)..(1): Xaa can be any naturally occurring amino acid
XSQPHTKPSVFVMKNGTNVACLVKEFYPKDIRINLVSSKKITEFDPAIVISPSGKYNAVKLGKYESNSVTCSVQHDNKTVHSTDFEVKTDSTDHVKPKETENTKQPSKSCHKPKAIVHTEKVNMMSLTVLGLRMLFAKTVAVNFLLTAKLFFL